(19) United States
(12) Patent Application Publication
(10) Pub. No.: US 2025/0002938 A1
(43) Pub. Date: Jan. 2, 2025

(54) VIRAL ADAPTORS AND USES THEREOF

(71) Applicant: THE FRANCIS CRICK INSTITUTE LIMITED, London (GB)

(72) Inventors: Samuel Gordon Rodriques, London (GB); Daniele Cervettini, London (GB); Bhuvana Sudarshan, London (GB)

(21) Appl. No.: 18/708,729

(22) PCT Filed: Nov. 7, 2022
(86) PCT No.: PCT/EP2022/080988

§ 371 Date: May 9, 2024

(30) Foreign Application Priority Data
Nov. 9, 2021 (GB) 2116101.3
Nov. 9, 2021 (GB) 2116104.7

(51) Int. Cl. C12N 15/86; C07K 14/705

(52) U.S. Cl. CPC C12N 15/86; C07K 14/705; C12N 2750/14122; C12N 2750/14143; C12N 2750/14145

(57) ABSTRACT

The present invention relates to viral adaptor proteins which are capable of changing the binding profile of the virus and/or modifying the cellular tropism of the virus. In one aspect, the present invention relates to a covalently-bound adaptor protein for viruses which is capable of changing the binding profile of the virus and/or modifying the cellular tropism of the virus. The present invention also relates to uses of the claimed virus adaptor proteins both to modify viral binding profiles and cellular tropism and as a transduction vector for use as a medicament.

The present invention relates to viral adaptor proteins which are capable of changing the binding profile of the virus and/or modifying the cellular tropism of the virus. In one aspect, the present invention relates to a covalently-bound adaptor protein for viruses which is capable of changing the binding profile of the virus and/or modifying the cellular tropism of the virus. The present invention also relates to uses of the claimed virus adaptor proteins both to modify viral binding profiles and cellular tropism and as a transduction vector for use as a medicament. In one aspect, the invention relates to an adaptor for adeno-associated viruses (AAVs) which is capable of changing the binding profile of the AAV and/or modifying the cellular tropism of the AAV. The present invention also relates to uses of the claimed AAV adaptors both to modify AAV binding profiles and cellular tropism and as a transduction vector for use as a medicament.

### BACKGROUND

### Adeno-Associated Viruses

The adeno-associated virus (AAV) virus belongs to the family of parvoviruses. These are distinguished by an icosahedral, non-enveloped capsid which has a diameter of 18 to 30 nm and which contains a linear, single-stranded DNA of about 5 kb. Efficient replication of AAV requires coinfection of the host cell with helper viruses, for example with adenoviruses, herpesviruses or vaccinia viruses. Alternatively, replication can be prompted in vitro in the absence of a helper virus if the viral proteins from the helper virus required to assist AAV replication are supplemented in isolation as part of a helper plasmid. This production method is the most common and is referred to as helper-free AAV production. In the absence of a helper virus, AAV enters a latent state, the viral genome being capable of stable integration into the host cell genome, although this happens rarely. The capacity of AAVs to infect a broad range of dividing and non-dividing cells, and its ability of viral genomes to either persist episomally within infected cells, or to integrate inside the host genome, makes it particularly interesting as a transduction vector for mammalian cells.

In general, the two inverted terminal repeats (ITR) which are about 145 bp long are sufficient for the vector functions. They carry the “cis” signals necessary for replication, packaging and integration into the host cell genome. For packaging in recombinant vector particles, a vector plasmid which carries the genes for non-structural proteins (Rep proteins) and for structural proteins (Cap proteins) is transfected into cells suitable for packaging, for example HeLa or HEK293 cells, which are then infected, for example, with adenovirus, or co-transfected with a helper plasmid.

The AAV capsid consists of three different proteins: VP1, VP2 and VP3, whose relative proportions are approximately 1:1:10 respectively, which equate to 8.3% VP1, 8.3% VP2 and 83.3% VP3. The AAV capsid genes are located at the end of the AAV genome and are encoded by the same open reading frame (ORF) using different start codons. The VP1 gene contains the whole VP2 gene sequence, which in turn contains the whole VP3 gene sequence with a specific N-terminal region. The fact that the one reading frame codes for all three AAV capsid proteins is responsible for the obligatory co-expression of all capsid proteins, although to different extents.

The molecular masses of the capsid proteins are 87 kDa for VP1, 73 kDa for VP2 and 62 kDa for VP3. The sequences of the capsid genes are described, for example, in Srivastava, A. et al. (1983), J. Virol., 45, 555-564; Muzyczka, N. (1992), Curr. Top. Micro. Immunol., 158, 97-129, Ruffing, N. et al. (1992), J. Virol., 66, 6922-6930 or Rutledge, E. A. et al. (1998) J. Virol. 72, 309-319, which are incorporated herein by reference in their entirety. The physical and genetic map of the AAV genome is described, for example, in Kotin, R. M. (1994), Human Gene Therapy, 5, 793-801, which is incorporated herein by reference in its entirety. Exemplary AAV capsid sequences are also provided herein in FIGS. 7 to 19 and SEQ ID NOs 7-19.

Also known are various AAV serotypes, of which the human AAV serotype 2 (AAV2) represents a virus vector with advantageous properties for somatic gene therapy. The essential advantages are the lack of pathogenicity for humans, the persistence of the viral genome as episome in the infected cells or its stable integration into the cellular genome, the ability to infect non-dividing cells, the stability of the virion, which makes purification to high titres possible, the low immunogenicity, and the substantial absence of viral genes and gene products in the recombinant AAV vector, which is advantageous from the viewpoint of safety for use in gene therapy. The cloning of genes into the AAV vector now takes place by methods generally known to the skilled person, as described, for example, in WO 95/23 867, in Chiorini J. A. et al. (1995), Human Gene Therapy, 6, 1531-1541 or in Kotin, R. M. (1994), which is incorporated herein by reference in its entirety.

AAV2 for example has in general a broad active spectrum (tropism). Epithelial tissues, such as human epithelial tumour cell lines, but also primary tumour material such as cervical or ovarian carcinoma or melanoma, and human keratinocytes are infected very efficiently (70-80%), whereas haematopoietic cells such as lymphohaemopoietic cells are infected with 10-to 100-fold lower efficiency (0.5-5%) (Mass et al. (1998) Human Gene Therapy, 9, 1049-1059, which is incorporated herein by reference in its entirety).

One reason for this might be that an interaction between AAV and an AAV receptor on the surface of the cell is necessary for uptake of AAV into the cell. Thus, for example, the putative primary AAV2 receptor is a cell membrane glycoprotein of 150 kDa (Mizukami, H. et al. (1996), Virology, 217, 124-130, which is incorporated herein by reference in its entirety) or heparan sulphate proteoglycan (Summerford, C. & Samulski, R. J. (1998), J. Virol., 72, 1438-1445, which is incorporated herein by reference in its entirety). Possible secondary receptors which have been determined are: α Vβ 5 integrin (Summerford et al., (1999), Nature Medicine 5, 78-82, which is incorporated herein by reference in its entirety) and human fibroblast growth factor receptor 1 (Qing et al., (1999) Nature Medicine 5, 71-77, which is incorporated herein by reference in its entirety). Binding studies have now shown that the surface density of this receptor is reduced on cells which are inefficiently infected by AAV2.

It is known that it is possible to introduce binding sites for receptors which are expressed only on particular cells by genetic modification of capsid proteins of retroviruses and adenoviruses, and thus a receptor-mediated targeting of vectors has been made possible (see, for example, Cosset, F. L. & Russell, S. J. (1996), Gene Ther., 3, 946-956, Douglas, J. T. et al. (1996), Nat. Biotechnol., 14, 1574-1578, Krasnykh, V. N. et al. (1996), J. Virol., 70, 6839-6846, Stevenson, S. C. et al. (1997), J. Virol., 71, 4782-4790 or Wickman, T. J. et al. (1996), Nat. Biotechnol., 14, 1570-1573, which are incorporated herein by reference in their entirety).

WO 96/00587 also refers to AVV capsid fusion proteins which are said to contain heterologous epitopes of clinically relevant antigens, which is said to induce an immune response, and which are said not to interfere with capsid formation. Steinbach et al. (1997) (Biol. Abstr. 104, Ref. 46570, which is incorporated herein by reference in its entirety) were concerned with the in vitro assembly of AAV particles which had previously been expressed in the baculo system. Mutations are also made on the cap gene, but these are intended not to lead to a change in the tropism but to a plasmid construct in which only one VP protein is expressed in each case. Ruffing et al. (1994) (J. Gen. Virol. 75, 3385-3392, which is incorporated herein by reference in its entirety) intended to investigate the natural tropism of AAV2. For this purpose, mutations were introduced at the C terminus of the AAV2 VP protein, the basic assumption being to change an RGD motif in this way.

Indirect targeting of AAVs is disclosed in Bartlett et al. (1999; Nat. Biotechnol. 17, 181-186, which is incorporated herein by reference in its entirety). In this case, there was use of a bispecific antibody which was directed both against the AAV2 capsid and against a target cell. Yang et al. (1998; Hum. Gene Ther. 1, 1929-1937, which is incorporated herein by reference in its entirety) discloses single-chain antibody fragments against the CD34 molecule fused to the N terminus of VP2, inserted directly at the N terminus of VP1. This method has, however, two distinct disadvantages. Firstly, the infection titre was very low and, secondly, for successful packaging it was necessary to co-express the fusion protein with unmutated capsid proteins VP1, VP2 and VP3. However, this resulted in a mixture of chimeric and wildtype capsid proteins, whose composition and thus activity was unpredictable. Moreover, the packaging efficiency and the infectivity via the wildtype receptor of HeLa cells was also considerably reduced compared with the wildtype.

One object of the present invention is therefore to modify AAVs in such a way that a more specific and more efficient gene transfer is possible than with known AAV vectors.

It has now been found, surprisingly, that AAV binding proteins, such as the AAV receptor (AAVR, KIAA0319L), or portions thereof, can be modified to provide robust binding to AAVs as part of a fusion protein, wherein the fusion partner can be used to change the tropism of the AAV. In some aspects, the binding protein can bind covalently to the virus. Similarly, other virus binding proteins can be modified to provide consistent covalent binding to virus capsids, as part of a fusion protein, wherein the fusion partner can be used to change the tropism of the virus.

### SUMMARY OF THE INVENTION

This summary introduces concepts that are described in more detail in the detailed description. It should not be used to identify essential features of the claimed subject matter, nor to limit the scope of the claimed subject matter.

The present invention provides an isolated polypeptide capable of binding to a viral capsid.

The present invention provides an isolated polypeptide comprising one or more cysteine residues capable of covalently binding to a viral capsid.

The present invention also provides a virus adaptor molecule comprising (i) an isolated polypeptide capable of binding to a viral capsid and (ii) a ligand.

The present invention also provides a virus adaptor molecule comprising (i) an isolated polypeptide comprising one or more cysteine residues capable of covalently binding to a viral capsid and (ii) a ligand.

In some embodiments, the one or more cysteine residues capable of covalently binding to a viral capsid are heterologous to the isolated polypeptide. In some embodiments, the virus adaptor protein comprises (i) an isolated polypeptide comprising one or more cysteine residues capable of covalently binding to a viral capsid and (ii) a ligand, wherein the one or more cysteine residues are heterologous to the isolated polypeptide. In some embodiments, the isolated polypeptide or covalent binding virus adaptor molecule is capable of binding to an unmodified, wildtype viral capsid.

In some embodiments, the isolated polypeptide or virus adaptor molecule is capable of binding to a viral capsid having 5 or fewer (for example 5, 4, 3, 2 or 1) amino acid point mutations compared to a wildtype viral capsid. In some embodiments, the isolated polypeptide or virus adaptor molecule is capable of binding to a viral capsid having an amino acid insertion or deletion of no more than 5 amino acids (i.e. 1, 2, 3, 4 or 5 amino acids) relative to a wildtype viral capsid. In some embodiments, the isolated polypeptide or virus adaptor molecule is capable of binding to a viral capsid having no insertions or deletions relative to a wildtype viral capsid.

In some embodiments, the isolated polypeptide or virus adaptor molecule comprises a portion of adeno-associated virus receptor (AAVR, KIAA0319L) capable of binding to an adeno associated virus capsid. In some embodiments, the isolated polypeptide or AAV adaptor molecule comprises the full-length sequence of adeno-associated virus receptor (AAVR, KIAA0319L).In some embodiments, the adeno-associated virus receptor (AAVR, KIAA0319L) sequence comprises the amino acid sequence of SEQ ID NO:1. In some embodiments, the isolated polypeptide or AAV adaptor molecule comprises one or more point mutations in the AAVR polypeptide, optionally wherein the one or more point mutations are defined by reference to SEQ ID NO:1.

In some embodiments, the isolated polypeptide or virus adaptor molecule comprises a portion of adeno-associated virus receptor (AAVR, KIAA0319L) having one or more cysteine residues and capable of covalently binding to an adeno associated virus capsid. In some embodiments, the isolated polypeptide or virus adaptor molecule comprises the full-length sequence of adeno-associated virus receptor (AAVR, KIAA0319L) having one or more cysteine residues and capable of covalently binding to an adeno associated virus capsid. In some embodiments, the adeno-associated virus receptor (AAVR, KIAA0319L) sequence comprises the amino acid sequence of SEQ ID NO:1. In some embodiments, the isolated polypeptide or covalent binding virus adaptor molecule comprises one or more point mutations in the AAVR polypeptide, optionally wherein the one or more point mutations are defined by reference to SEQ ID NO:1.

In some embodiments, the isolated polypeptide or covalent binding virus adaptor molecule comprises one or more point mutations in the AAVR polypeptide to introduce one or more heterologous cysteine residues, optionally wherein the one or more point mutations are defined by reference to SEQ ID NO:1.

In some embodiments, the isolated polypeptide or virus adaptor molecule comprises a portion of a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof. In some embodiments the isolated polypeptide or AAV adaptor molecule comprises an antibody or antigen binding fragment thereof.

In some embodiments, the isolated polypeptide or virus adaptor molecule comprises a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof. In some embodiments the isolated polypeptide or AAV adaptor molecule comprises an antibody or antigen binding fragment thereof.

In some embodiments, the isolated polypeptide or virus adaptor molecule comprises an antigen binding portion of neutralising antibody A20. In some embodiments, the isolated polypeptide or virus adaptor molecule comprises a sequence substantially identical to one or more complementarity determining regions (CDRs) of neutralising antibody A20, for example wherein the one or more complementarity determining regions (CDRs) of neutralising antibody A20 may comprise 1, 2 or 3 amino acid point mutations, optionally wherein one or more of the point mutations is a heterologous cysteine residue. In some embodiments, the isolated polypeptide or virus adaptor molecule comprises one or more sequences that are substantially identical to each of the complementarity determining regions (CDRs) of neutralising antibody A20, for example wherein 1, 2, 3, 4, 5 or 6 of the complementarity determining regions (CDRs) of neutralising antibody A20 may comprise 1, 2 or 3 amino acid point mutations, optionally wherein one or more of the point mutations is a heterologous cysteine residue.

In some embodiments, the isolated polypeptide or virus adaptor molecule comprises the full-length sequence of one or more complementarity determining regions (CDRs) of neutralising antibody A20, for example wherein the polypeptide comprises one or more of VHCDR1 of antibody A20 (SEQ ID NO:26), VHCDR2 of antibody A20 (SEQ ID NO:27), VHCDR3 of antibody A20 (SEQ ID NO:28), VLCDR1 of antibody A20 (SEQ ID NO:22), VLCDR2 of antibody A20 (SEQ ID NO:23) and VLCDR3 of antibody A20 (SEQ ID NO:24).

In some embodiments, the isolated polypeptide or virus adaptor molecule comprises all of the complementarity determining regions (CDRs) of neutralising antibody A20. In some embodiments, the isolated polypeptide or virus adaptor molecule comprises one or more sequences that are substantially identical to the full-length VH chain (SEQ ID NO:25) and VL chain (SEQ ID NO:21) sequences of neutralising anti-body A20, for example wherein the VH and/or VL sequences of neutralising antibody A20 may comprise 1, 2, 3, 4, 5, 6, 7, 8, 9 or 10 amino acid point mutations, optionally wherein one or more of the point mutations is a heterologous cysteine residue.

In some embodiments, the isolated polypeptide or virus adaptor molecule comprises the full-length VH chain (SEQ ID NO:25) and VL chain (SEQ ID NO:21) sequences of neutralising antibody A20.

In some embodiments, the isolated polypeptide or virus adaptor molecule further comprises one or more of:
one or more non-natural amino acids,
one or more chemical moieties cross linked to the polypeptide,
a biotin tag, for example biotinylated AAVR,
a SpyTag peptide, for example AAVR-SpyTag; and/or
a Protein A or Protein G polypeptide.

In some embodiments, the ligand is capable of binding to a cell surface molecule. In some embodiments, the ligand is a human protein, such as an antibody or antigen binding fragment thereof. In some embodiments, the ligand is one or more ligands selected from the list consisting of: Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Interleukin-17, Interleukin-18, Interleukin-19, Interleukin-20, Interleukin-21, Interleukin-22, Interleukin-23, Interleukin-24, Interleukin-25, Interleukin-26, Interleukin-27, Interleukin-28, Interleukin-29, Interleukin-30, Interleukin-31, Interleukin-32, Interleukin-33, Interleukin-34, Interleukin-35, Insulin, Transferrin, CD2, CD58, CD59, CD2, CD40L/CD154, CD5, CD72, CD5L, CD23, CD70, CD80, CD86, S100Ap, CD178, CD155, CD106, CSF1, CD166, FasL, CD242, CD252, TRAIL, RANKL, APRIL, CD257, CD272, CD273, CD274, CD275, PD-L1, PD-L2, Cas13 and Cas7-11, endothelin, leptin, vasopressin, CD10, CD31, CD119, apelin, elabela, adrenomedullin, a targeting domain from botulinum toxin, a neuropeptide, a cytokine or a small molecule.

In some embodiments, the ligand is a small molecule, optionally wherein the small molecule is linked to the isolated polypeptide or virus adaptor molecule via N-Hydroxysuccinimide (NHS) or maleimide. In some embodiments, the ligand is a small molecule, optionally wherein the isolated polypeptide or virus adaptor molecule comprises a noncanonical amino acid that links the small molecule to the isolated polypeptide or virus adaptor molecule.

In some embodiments, the ligand binds one or more cell surface molecules selected from the list consisting of: Her2, Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-8 receptor, Interleukin-9 receptor, Interleukin-10 receptor, Interleukin-11 receptor, Interleukin-12 receptor, Interleukin-13 receptor, Interleukin-15 receptor, Interleukin-18 receptor, Interleukin-receptor, Interleukin-21 receptor, Interleukin-22 receptor, Interleukin-23 receptor, Interleukin-27 receptor, Interleukin-28 receptor, Insulin receptor, Transferrin receptor, CD58, CD2, CD2, CD59, CD40, CD72, CD5, CD36, CD19, CD21, CD81, CD27, CD28, CTLA-4, CD85j, CD95, CD96, α4β1 integrin, CD115, CD6, CD178, LFA-1, TNFRSF4, DR4, DR5, RANK/CD265, TACI/CD267, CD267, CD268, CD269, HVEM, PD1/CD279, B7-1/CD80, CD278, CD4, CD8, CD19, NMDAR, AMPAR, mGluR5, DRD1, DRD2, Bmp4, GLP1R, leptin receptor, α5β5 integrin and glycoRNAs.

In some embodiments, the portion of adeno-associated virus receptor (AAVR, KIAA0319L) comprises at least a portion of the PKD1 domain, at least a portion of the PKD2 domain, at least a portion of the PKD3 domain, at least a portion of the PKD4 domain, at least a portion of the PKD5 domain, or any combination thereof.

In some embodiments, the portion of adeno-associated virus receptor (AAVR, KIAA0319L) comprises the PKD1 domain, the PKD2 domain, the PKD3 domain, the PKD4 domain, the PKD5 domain, or any combination thereof. The isolated polypeptide or covalent binding virus adaptor molecule according to any one of claims 9 to 30, wherein the portion of adeno-associated virus receptor (AAVR, KIAA0319L) comprises a sequence selected from the list consisting of: SEQ ID NO:2, SEQ ID NO:3, SEQ ID NO:4, SEQ ID NO:5 and SEQ ID NO:6.

In some embodiments, the portion of adeno-associated virus receptor (AAVR, KIAA0319L) comprises the full-length sequence of adeno-associated virus receptor (AAVR, KIAA0319L), optionally wherein the full-length sequence comprises SEQ ID NO:1.

The present invention also provides a virus particle bound to at least one isolated polypeptide or virus adaptor molecule according to the invention. In some embodiments the virus particle is covalently bound to at least one isolated polypeptide or virus adaptor molecule according to the invention.

In some embodiments, one or more viral capsid proteins comprise one or more cysteine residues capable of covalently binding to an isolated polypeptide. In some embodiments, one or more viral capsid proteins comprise one or more cysteine residues capable of forming a disulphide bridge with one or more cysteine residues in an isolated polypeptide.

In some embodiments, the virus is an adeno-associated virus, optionally wherein the adeno associated virus is selected from the list consisting of: AAV1, AAV2, AAV3, AAV4, AAV5, AAV6, AAV7, AAV8, AAV9, AAV10, AAV11, AAV12, AAV13, AAVrh.8, AAVrh.10, AAVrh.74. In some embodiments, the adeno-associated virus is AAV1. In some embodiments, the adeno-associated virus is AAV2. In some embodiments, the adeno-associated virus is a wildtype AAV.

In some embodiments, the adeno-associated virus comprises a portion of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.

In some embodiments, the adeno-associated virus comprises the full-length of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.

In some embodiments, the adeno-associated virus has one or more conservative amino acid changes relative to the wildtype amino acid sequence, for example 1, 2, 3, 4, 5, 6, 7, 8, 9, 10, 11, 12, 13, 14, 15, 16, 17, 18, 19 or 20 conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19. In some embodiments, the adeno-associated virus has five or fewer non-conservative amino acid changes relative to the wildtype amino acid sequence, for example 5, 4, 3, 2 or 1 non-conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.

In some embodiments, the virus is selected from the list consisting of: Adenovirus, Aichi virus, Australian bat lyssavirus, BK polyomavirus, Banna virus, Barmah forest virus, Bunyamwera virus, Bunyavirus, La CrosseBunyavirus, snowshoe hare Cercopithecine herpesvirus, Chandipura virus, Chikungunya virus, Cosavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus, C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human herpesvirus, Human immunodeficiency virus, Human papillomavirus, Human papillomavirus, Human parainfluenza, Human parvovirus, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New Jersey polyomavirus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus, ARoss river virus, Rotavirus, ARotavirus, BRotavirus, CRubella virus, Sagiyama virus, Salivirus, ASandfly fever sicilian virus, Sapporo virus, SARS coronavirus, Semliki forest virus, Seoul virus, Simian foamy virus, Simian virus 40, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus and Zika virus. In some embodiments, the virus is a wildtype virus.

In some embodiments, the virus is an adenovirus and the isolated polypeptide capable of binding to a viral capsid or the virus adaptor molecule comprising an isolated polypeptide capable of binding to a viral capsid and a ligand comprises a portion of the coxsackievirus-adenovirus receptor (CXADR), optionally wherein the CXADR sequence is SEQ ID NO:20.

In some embodiments, the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a portion of the coxsackievirus-adenovirus receptor (CXADR), optionally wherein the CXADR sequence is SEQ ID NO:20.

In some embodiments, the virus is an adenovirus and the isolated polypeptide capable of binding to a viral capsid or the virus adaptor molecule comprising an isolated polypeptide capable of binding to a viral capsid and a ligand comprises a portion of CD46, optionally wherein the CD46 sequence is SEQ ID NO:29.

In some embodiments, the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a portion of CD46, optionally wherein the CD46 sequence is SEQ ID NO:29.

In some embodiments, the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a full-length sequence of a coxsackievirus-adenovirus receptor (CXADR), optionally wherein the CXADR sequence is SEQ ID NO:20. In some embodiments, one or more virus capsids have one or more heterologous cysteine residues introduced relative to the wildtype amino acid sequence, for example 1, 2, 3, 4, 5, 6, 7, 8, 9 or 10 heterologous cysteine residues relative to the wildtype amino acid sequence. In some embodiments, the one or more heterologous cysteine residues are introduced at the interface between the isolated polypeptide or virus adaptor molecule and the virus capsid.

In some embodiments, the virus is AAV1 and the isolated polypeptide or an isolated fusion polypeptide comprises a portion of adeno-associated virus receptor (AAVR, KIAA0319L) capable of covalently binding to an adeno associated virus capsid, and wherein the AAV1 comprises one or more mutations selected from Gly266Cys, Thr504Cys and Asp590Cys, and wherein the AAVR polypeptide or fusion polypeptide comprises one or more mutations selected from Thr434Cys, Asp429Cys and Ser425Cys.

In some embodiments, the virus is AAV2 and the isolated polypeptide or an isolated fusion polypeptide comprises a portion of adeno-associated virus receptor (AAVR, KIAA0319L) capable of covalently binding to an adeno associated virus capsid, and wherein the AAV2 comprises one or more mutations selected from Gly265Cys, Thr503Cys and Gln589Cys, and wherein the AAVR polypeptide or fusion polypeptide comprises one or more mutations selected from Thr434Cys, Asp429Cys and Ser425Cys.

In some embodiments, the virus is AAV2 and the isolated polypeptide or an isolated fusion polypeptide comprises a portion of adeno-associated virus receptor (AAVR, KIAA0319L) capable of covalently binding to an adeno associated virus capsid, and wherein the AAV2 comprises a Gln589Cys mutation, and wherein the AAVR polypeptide or fusion polypeptide comprises one or more mutations selected from Ser425Cys and Val480Glu, optionally wherein the AAVR polypeptide or fusion polypeptide comprises both Ser425Cys and Val480Glu mutations.

In some embodiments, the virus is AAV2 and the isolated polypeptide or an isolated fusion polypeptide comprises a PKD2 domain (e.g. SEQ ID NO:3) of adeno-associated virus receptor (AAVR, KIAA0319L) capable of covalently binding to an adeno associated virus capsid, and wherein the AAV2 comprises a Gln589Cys mutation, and wherein the AAVR polypeptide or fusion polypeptide comprises one or more mutations selected from Ser425Cys and Val480Glu, optionally wherein the AAVR polypeptide or fusion polypeptide comprises both Ser425Cys and Val480Glu mutations.

In some embodiments, the virus is AAV1 and the isolated polypeptide or an isolated fusion polypeptide comprises a portion of adeno-associated virus receptor (AAVR, KIAA0319L) capable of covalently binding to an adeno associated virus capsid, and wherein the AAV2 comprises a Gln589Cys mutation, and wherein the AAVR polypeptide or fusion polypeptide comprises one or more mutations selected from Ser425Cys and Val480Glu, optionally wherein the AAVR polypeptide or fusion polypeptide comprises both Ser425Cys and Val480Glu mutations.

In some embodiments, the virus is AAV1 and the isolated polypeptide or an isolated fusion polypeptide comprises a PKD2 domain (e.g. SEQ ID NO:3) of adeno-associated virus receptor (AAVR, KIAA0319L) capable of covalently binding to an adeno associated virus capsid, and wherein the AAV2 comprises a Gln589Cys mutation, and wherein the AAVR polypeptide or fusion polypeptide comprises one or more mutations selected from Ser425Cys and Val480Glu, optionally wherein the AAVR polypeptide or fusion polypeptide comprises both Ser425Cys and Val480Glu mutations.

In some embodiments, the virus is AAV5 and the isolated polypeptide or an isolated fusion polypeptide comprises a portion of adeno-associated virus receptor (AAVR, KIAA0319L) capable of covalently binding to an adeno associated virus capsid, and wherein the AAV5 comprises a Gln697Cys mutation, and wherein the AAVR polypeptide or fusion polypeptide comprises a Ser356Cys mutation.

In some embodiments, the virus is AAV5 and the isolated polypeptide or an isolated fusion polypeptide comprises a PKD1 domain (e.g. SEQ ID NO:2) of adeno-associated virus receptor (AAVR, KIAA0319L) capable of covalently binding to an adeno associated virus capsid, and wherein the AAV5 comprises a Gln697Cys mutation, and wherein the AAVR polypeptide or fusion polypeptide comprises a Ser356Cys mutation.

In some embodiments, the virus is AAV2 and the isolated polypeptide or an isolated fusion polypeptide comprises an antigen binding portion of neutralising antibody A20, and wherein the AAV2 comprises one or more mutations selected from Ser264Cys, Val708Cys and Asn717Cys, and wherein the neutralising antibody A20 polypeptides or fusion polypeptides comprise one or more mutations selected from VH Tyr102Cys, VH Ser56Cys and VL Ile93Cys.

In some embodiments, the virus is AAV2 and the isolated polypeptide or an isolated fusion polypeptide comprises an antigen binding portion of neutralising antibody A20, and wherein the AAV2 comprises one or more mutations selected from Gly266Cys, Thr504Cys and Asp590Cys, and wherein the neutralising antibody A20 polypeptides or fusion polypeptides comprise one or more mutations selected from VH Tyr102Cys, VH Ser56Cys and VL Ile93Cys.

In some embodiments, the adeno-associated virus particle comprises one or more amino acid changes in the capsid protein that mediates the interaction with non-protein binders such as heparan sulfate proteoglycan (HSPG), O-linked sialic acid, N-linked sialic acid, N-linked galactose.

In some embodiments, the adeno-associated virus particle comprises mutations at arginine residues 585 and 588 of AAV2.

The present invention also provides a pharmaceutical composition comprising a virus particle according to the invention.

The present invention also provides a method of covalently modifying a virus particle comprising:
providing a virus particle;
providing an isolated polypeptide capable of covalently binding to a virus capsid of the virus particle; and
combining the virus particle and the isolated polypeptide such that the isolated polypeptide covalently binds to the virus particle.

The present invention also provides a method of covalently modifying a virus particle comprising:
providing a virus particle;
providing a virus adaptor molecule comprising an isolated polypeptide capable of covalently binding to a virus capsid of the virus particle and a ligand; and
combining the virus particle and the virus adaptor molecule such that the isolated fusion polypeptide covalently binds to the virus particle.

In some embodiments, the method further comprises introducing one or more heterologous cysteine residues to the isolated polypeptide or virus adaptor molecule, for example 1, 2 or 3 heterologous cysteine residues. In some embodiments, the method further comprises introducing one or more heterologous cysteine residues to the virus capsid, for example 1, 2 or 3 heterologous cysteine residues.

In some embodiments, the method further comprises introducing one or more heterologous cysteine residues to both the isolated polypeptide or virus adaptor molecule and to the virus capsid, for example 1, 2 or 3 heterologous cysteine residues to both the isolated polypeptide or virus adaptor molecule and to the virus capsid.

In some embodiments, the binding of the isolated polypeptide or virus adaptor molecule to the virus particle reduces or abolishes the natural tropism of one or more virus capsid proteins. In some embodiments, the binding of the isolated polypeptide or virus adaptor molecule to the virus particle increases the tropism of the virus particle for one or more cell types.

In some embodiments, the binding of the isolated polypeptide or virus adaptor molecule to the virus particle increases and/or reduces the tropism of the virus particle for one or more cell types selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.

In some embodiments, the virus particle has reduced or increased tropism compared to the natural tropism of a virus particle not comprising the isolated polypeptide or the virus adaptor molecule of at least 10%, for example at least 20%, at least 30%, at least 40%, at least 50%, at least 60%, at least 70%, at least 80%, at least 90% or at least 100%. In some embodiments, the binding of the isolated polypeptide or virus adaptor molecule to the virus particle allows the virus particle to display protein antigens in a multimeric fashion using the virus as a scaffold.

In some embodiments, the covalent binding of the isolated polypeptide or virus adaptor molecule to the virus particle selectively induces cell lysis, optionally wherein cell lysis in induced in one or more cell types selected from the list consisting of: neurons, macrophages, microglia, T-cells, B-cells, dendritic cells, antigen presenting cells, NK cells, cancer cells, muscle cells, hepatocytes, photoreceptors, pancreatic beta cells and lung cells.

The present invention also provides a method of targeting a virus particle to a target cell comprising:
providing a virus particle;
providing a virus adaptor molecule comprising an isolated polypeptide capable of binding to a virus capsid of the virus particle and a ligand specific to the target cell;
combining the virus particle and the virus adaptor molecule such that the virus adaptor molecule binds to the virus particle, thereby generating a modified virus particle; and
contacting a mixture of cells comprising the target cell with the modified virus particle.

In some embodiments of methods of the invention, the isolated polypeptide capable of binding to an adeno associated virus (AAV) capsid comprises a portion of adeno-associated virus receptor (AAVR, KIAA0319L). In some embodiments of methods of the invention, the isolated polypeptide capable of binding to an adeno associated virus (AAV) capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination thereof. of methods of the invention, the isolated polypeptide capable of binding to an adeno associated virus (AAV) capsid comprises the full PKD1 domain, the full PKD2 domain, the full PKD3 domain, the full PKD4 domain, the full PKD5 domain of adeno-associated virus receptor (AAVR, KIAA0319L), or any combination thereof. In some embodiments, the isolated polypeptide capable of binding to an adeno associated virus (AAV) capsid comprises one or more mutations in the AAVR polypeptide, optionally wherein the one or more mutations are defined by reference to SEQ ID NO:1 isolated polypeptide capable of binding to an adeno associated virus (AAV) capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination there-of and wherein the isolated polypeptide capable of binding to an adeno associated virus (AAV) capsid comprises one or more mutations in the AAVR polypeptide, optionally wherein the one or more mutations are defined by reference to SEQ ID NO:1. In some embodiments, the isolated polypeptide capable of binding to an adeno associated virus (AAV) capsid comprises the full PKD1 domain, the full PKD2 domain, the full PKD3 domain, the full PKD4 domain, the full PKD5 domain of adeno-associated virus receptor (AAVR, KIAA0319L), or any combination thereof and wherein the isolated polypeptide capable of binding to an adeno associated virus (AAV) capsid comprises one or more mutations in the AAVR polypeptide, optionally wherein the one or more mutations are defined by reference to SEQ ID NO:1.

In some embodiments of methods of the invention, the isolated polypeptide capable of covalently binding to a virus capsid comprises a portion of adeno-associated virus receptor (AAVR, KIAA0319L). In some embodiments of methods of the invention, the isolated polypeptide capable of covalently binding to a virus capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination thereof. In some embodiments of methods of the invention, the isolated polypeptide capable of covalently binding to a virus capsid comprises the PKD1 domain, the PKD2 domain, the PKD3 domain, the PKD4 domain, the PKD5 domain of adeno-associated virus receptor (AAVR, KIAA0319L), or any combination thereof.

In some embodiments of methods of the invention, the isolated polypeptide capable of covalently binding to an adeno associated virus (AAV) capsid comprises a sequence selected from the list consisting of: SEQ ID NO:2, SEQ ID NO:3, SEQ ID NO:4, SEQ ID NO:5 and SEQ ID NO:6. In some embodiments of methods of the invention, the isolated polypeptide capable of binding to a virus capsid comprises the full-length sequence of adeno-associated virus receptor (AAVR, KIAA0319L), optionally wherein the full sequence comprises SEQ ID NO:1.

The present invention also provides a method of delivering a nucleic acid sequence of interest to a target cell comprising contacting the target cell with the modified virus particle according to the invention, or the pharmaceutical composition according to the invention, wherein the virus particle comprises the nucleic acid sequence of interest.

In some embodiments of methods of the invention, the nucleic acid sequence of interest encodes a protein selected from the list consisting of:
RAB escort protein 1, RPE65, Factor VIII, Factor IX, Cochlin, CLN7, acid a-glucosidase (GAA), Aquaporin 1, Glial cell line-derived neurotrophic factor, aspartoacylase, Aromatic L-amino Acid Decarboxylase, Defects in Retinitis Pigmentosa GTPase Regulator, sarcoplasmic reticulum calcium ATPase, Cyclic nucleotide gated channel beta 3, neurturin, Galactosidase beta 1, Glucose-6-Phosphatase, Phenylalanine hydroxylase, Ornithine Transcarbamylase, Dystrophin and Carnitine palmitoyltransferase II, phenylalanine hydroxylase (PAH), Cystic fibrosis transmembrane conductance regulator (CFTR).

In some embodiments, the target cell is in vitro. In some embodiments, the target cell is in vivo in a subject. In some embodiments, the target cell is selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells. In some embodiments, the subject is a human. In some embodiments, the target cell is a human target cell.

The present invention also provides a virus particle according to the invention or a pharmaceutical composition according to the invention for use as a medicament. The present invention also provides a method of treating a patient in need thereof comprising administering a therapeutically effective amount of a virus particle according to the invention or a pharmaceutical composition according to the invention.

The present invention also provides a virus particle according to the invention, or a pharmaceutical composition according to the invention for use in a method of treating a disease in a subject in need thereof, the method comprising delivering a nucleic acid sequence of interest to a target cell by contacting the target cell with the virus particle. The present invention also provides a method of treating a disease in a subject in need thereof, the method comprising delivering a nucleic acid sequence of interest to a target cell by administering a therapeutically effective amount of a virus particle according to the invention, or a pharmaceutical composition according to the invention, thereby contacting the target cell with the virus particle.

In some embodiments, the disease is selected from the list consisting of:
a neurodegenerative disorder, cancer, Duchenne muscular dystrophy, haemophilia, a congenital blindness disorder, diabetes, cystic fibrosis, choroideremia, hemophilia A, hemophilia B, CLN7 disease, Pompe disease, Parkinson's disease, Canavan disease, demyelinating diseases, inherited retinal dystrophy due to RPE65 mutations, aromatic L-amino acid decarboxylase (AADC) deficiency, X-linked retinitis pigmentosa, Leber congenital amaurosis, Churg-Strauss Syndrome (CSS), critical limb ischemia, achromatopsia, Alzheimer's disease, macular degeneration, ornithine transcarbamylase deficiency, Wilson disease, glycogen storage disease type IA, Crigler Najjar syndrome, Tay-Sachs disease, Sandhoff disease, multiple myeloma, multiple system atrophy, gangliosidosis, Danon disease, Fabry disease, Batten disease, phenylketonuria, rheumatoid arthritis, mucopolysaccharidosis type IIIa, Sanfilippo syndrome B, mucopolysaccharidosis type VI, alpha 1-antitrypsin deficiency, spinal muscular atrophy type 1, Krabbe disease, Becker muscular dystrophy, Charcot-Marie-Tooth neuropathy type 1a, carnitine palmitoyltransferase II (CPT II) deficiency and trimethylaminuria, cystic fibrosis, phenylketonuria.

In some embodiments of the invention, the virus particles or compositions of the invention are for use in a method of treating a disease in a subject in need thereof, the method comprising delivering a nucleic acid sequence of interest to a target cell by contacting the target cell with the virus particle, wherein the targeting of the specified target cell treats or ameliorates the specified disease according to the following list:
neurons for treatment of neurodegenerative disorders;
immune cells (e.g. macrophages, microglia, T-cells, B-cells, dendritic cells, antigen presenting cells, NK cells) for treatment of cancer;
immune cells (e.g. macrophages, microglia, T-cells, B-cells, dendritic cells, antigen presenting cells, NK cells) to elicit an immune response;
cancer or tumour cells for treatment of cancer;
muscle cells for treatment of Duchenne muscular dystrophy;
hepatocytes for treatment of haemophilia;
photoreceptor cells for congenital blindness disorders;
pancreatic beta cells for treatment of diabetes; or
lung cells for treatment of cystic fibrosis.

In some embodiments, the virus particle or a pharmaceutical composition is administered to the subject via aerosol (e.g to lung cells), intramuscularly, intraarterially (e.g. via the hepatic artery), intraarticularly, subretinally, intracranially, intravenously, intrathecally. intra-coronarily or subcutaneously.

In some embodiments of the invention, the ligand may consist of a sequence-specific RNA binding molecule, such as a Cas13 molecule. This would then endow the virus with tropism towards cells that express the target RNA on their surfaces. Specifically, in this case, the adaptor molecule would consist of a fusion of Cas13 to a portion of, or the full-length PKD2 domain. The Cas13 could then form a complex with a single guide RNA (sgRNA) appropriate for Cas13, endowing it with the ability to bind RNAs that are complementary to the protospacer region of the sgRNA.

In some embodiments of the invention, a method of generating a viral adaptor protein comprises providing a viral capsid protein and reacting it with a targeting molecule with high specificity for a target cell. The reaction may be performed using NHS or maleimide chemistry, or it may be performed by incorporating a non-canonical amino acid with a reactive group (such as an azide or an alkyne or a tetrazine or a trans-cyclooctene) at a specific location in the viral capsid molecule. In some embodiments of the invention, a viral adaptor protein comprises a viral capsid protein and a targeting molecule with high specificity for a target cell.

In some embodiments, the viral capsid molecule is combined with a specific peptide sequence that is capable of binding to the targeting molecule, such as a split intein, SpyTag, the tetracysteine FCM motif (FLNCCPGCCMEP) or the ybbR motif (TVLDSLEFIASKLA).

The targeting molecule may then be chosen to bind to a specific receptor. In some embodiments, the targeting molecule may be a protein, such as an antibody against a receptor expressed on the target cell type.

In some embodiments, the targeting molecule may be a small molecule. For example, by attaching a neurotransmitter such as serotonin or dopamine to the viral capsid protein, it would be possible to create an adaptor molecule that, when combined with a virus particle, would generate a virus particle specific to cells that express the serotonin or dopamine receptors, respectively.

In some embodiments, an AAV particle may be bound to more than one isolated polypeptide or AAV adaptor molecule according to the invention. In some embodiments, an AAV particle may be bound to 2, 3, 4, 5, 6, 7, 8, 9, 10 or more different isolated polypeptides or different AAV adaptor molecules according to the invention. In some embodiments, an AAV particle may be bound to 2 different isolated polypeptides or different AAV adaptor molecules according to the invention. In some embodiments, an AAV particle may be bound to 3 different isolated polypeptides or different AAV adaptor molecules according to the invention. In some embodiments, an AAV particle may be bound to 4 different isolated polypeptides or different AAV adaptor molecules according to the invention. In some embodiments, an AAV particle may be bound to 5 different isolated polypeptides or different AAV adaptor molecules according to the invention. Such an embodiment provides specificity for multiple different cell types. For example, a single AAV-based gene therapy could be formulated to target both CD4+ and CD8+ T cell lineages by combining a single AAV particle with a mixture of adaptor molecules, some of which are specific for CD4 and some of which are specific for CD8.

In some embodiments, a virus particle may be covalently bound to more than one isolated polypeptide or virus adaptor molecule according to the invention. In some embodiments, a virus particle may be covalently bound to 2, 3, 4, 5, 6, 7, 8, 9, 10 or more different isolated polypeptides or different virus adaptor molecules according to the invention. In some embodiments, a virus particle may be covalently bound to 2 different isolated polypeptides or different virus adaptor molecules according to the invention. In some embodiments, a virus particle may be covalently bound to 3 different isolated polypeptides or different virus adaptor molecules according to the invention. In some embodiments, a virus particle may be covalently bound to 4 different isolated polypeptides or different virus adaptor molecules according to the invention. In some embodiments, a virus particle may be covalently bound to 5 different isolated polypeptides or different virus adaptor molecules according to the invention. Such an embodiment provides specificity for multiple different cell types. For example, a single virus gene therapy could be formulated to target both CD4+ and CD8+ T cell lineages by combining a single virus particle with a mixture of adaptor molecules, some of which are specific for CD4 and some of which are specific for CD8.

In some embodiments, an AAV particle may be bound to more than one isolated polypeptide or AAV adaptor molecule according to the invention. In some embodiments, an AAV particle may be bound to 2, 3, 4, 5, 6, 7, 8, 9, 10 or more different isolated polypeptides or different AAV adaptor molecules according to the invention. In some embodiments, an AAV particle may be bound to 2 different isolated polypeptides or different AAV adaptor molecules according to the invention. In some embodiments, an AAV particle may be bound to 3 different isolated polypeptides or different AAV adaptor molecules according to the invention. In some embodiments, an AAV particle may be bound to 4 different isolated polypeptides or different AAV adaptor molecules according to the invention. In some embodiments, an AAV particle may be bound to 5 different isolated polypeptides or different AAV adaptor molecules according to the invention. Such an embodiment provides specificity for multiple different cell types. For example, a single AAV gene therapy could be formulated to target both CD4+ and CD8+ T cell lineages by combining a single AAV particle with a mixture of adaptor molecules, some of which are specific for CD4 and some of which are specific for CD8.

In some embodiments, the isolated polypeptide or virus adaptor molecule of the invention comprises at least a portion of a CD4-specific Designed Ankyrin Repeat Protein (DARPin) as disclosed in Schweizer A, Rusert P, Berlinger L, Ruprecht C R, Mann A, Corthésy S, et al. (2008) CD4-Specific Designed Ankyrin Repeat Proteins Are Novel Potent HIV Entry Inhibitors with Unique Characteristics. PLoS Pathog 4(7): e1000109. In some embodiments, the isolated polypeptide or virus adaptor molecule of the invention comprises the full-length of a CD4-specific Designed Ankyrin Repeat Protein (DARPin) as disclosed in Schweizer A, Rusert P, Berlinger L, Ruprecht C R, Mann A, Corthésy S, et al. (2008) CD4-Specific Designed Ankyrin Repeat Proteins Are Novel Potent HIV Entry Inhibitors with Unique Characteristics. PLoS Pathog 4(7): e1000109.

In some embodiments of the invention, the isolated polypeptide or virus adaptor molecule of the invention comprises at least a portion of any suitable antibody-like protein capable of binding a virus capsid (Kondo T et al., Antibody-like proteins that capture and neutralize SARS-CoV-2. Sci Adv. 2020 Oct. 14; 6(42):eabd3916). In some embodiments of the invention, the isolated polypeptide or virus adaptor molecule of the invention comprises a full-length protein of any suitable antibody-like protein capable of binding a virus capsid.

In some embodiments of the invention, the isolated polypeptide or virus adaptor molecule of the invention comprises at least a portion of a protein derived from the lipocalin fold capable of binding a virus capsid (Beste et al., Small antibody-like proteins with prescribed ligand specificities derived from the lipocalin fold, PNAS Mar. 2, 1999 96 (5) 1898-1903). In some embodiments of the invention, the isolated polypeptide or virus adaptor molecule of the invention comprises a full-length protein derived from the lipocalin fold capable of binding a virus capsid.

In some embodiments of the invention, the isolated polypeptide or virus adaptor molecule of the invention comprises at least a portion of an affibody derived from the Z domain of Protein A capable of binding a virus capsid (Nord et al., Binding proteins selected from combinatorial libraries of an a-helical bacterial receptor domain, Nature Biotechnology, volume 15, pages 772-777 (1997)). In some embodiments of the invention, the isolated polypeptide or virus adaptor molecule of the invention comprises a full-length affibody derived from the Z domain of Protein A capable of binding a virus capsid.

In some embodiments of the invention, the isolated polypeptide or virus adaptor molecule of the invention comprises at least a portion of a domain of fibronectin capable of binding a virus capsid (Koide, et al., The fibronectin type Ill domain as a scaffold for novel binding proteins, Journal of Molecular Biology, Volume 284, Issue 4, 1998, Pages 1141-1151). In some embodiments of the invention, the isolated polypeptide or virus adaptor molecule of the invention comprises the full-length sequence of a domain of fibronectin capable of binding a virus capsid.

In some embodiments of the invention, the isolated polypeptide or virus adaptor molecule of the invention comprises at least a portion of an SH3 domain of Fyn capable of binding a virus capsid (Grabulovski, et al., A Novel, Non-immunogenic Fyn SH3-derived Binding Protein with Tumor Vascular Targeting Properties, Journal of Biological Chemistry, Volume 282, Issue 5, 2007, Pages 3196-3204). In some embodiments of the invention, the isolated polypeptide or virus adaptor molecule of the invention comprises the full-length sequence of an SH3 domain of Fyn capable of binding a virus capsid.

In some embodiments of the invention, the isolated polypeptide or virus adaptor molecule of the invention comprises at least a portion of a derivative of an affimer or a scaffold derived from the protease inhibitor stefin A capable of binding a virus capsid (Woodman R, Yeh J T, Laurenson S, Ko Ferrigno P. Design and validation of a neutral protein scaffold for the presentation of peptide aptamers. J Mol Biol. 2005 Oct. 7; 352(5):1118-33). In some embodiments of the invention, the isolated polypeptide or virus adaptor molecule of the invention comprises the full-length sequence of a derivative of an affimer or a scaffold derived from the protease inhibitor stefin A capable of binding a virus capsid.

In some embodiments of the invention, the isolated polypeptide or virus adaptor molecule of the invention comprises at least a portion of a non-antibody scaffold protein (Adhiron) capable of binding a virus capsid (Tiede et al., Adhiron: a stable and versatile peptide display scaffold for molecular recognition applications, Protein Eng Des Sel. 2014 May; 27(5): 145-155). In some embodiments of the invention, the isolated polypeptide or virus adaptor molecule of the invention comprises the full-length sequence of a non-antibody scaffold protein (Adhiron) capable of binding a virus capsid.

In some embodiments, the isolated polypeptide capable of binding to an adeno-associated virus (AAV) capsid or AAV adaptor molecule comprising an isolated polypeptide capable of binding to an adeno associated virus (AAV) capsid and a ligand comprises one or more point mutations that increase the solubility of the isolated polypeptide or adaptor molecule, for example 1, 2, 3, 4, 5 or more point mutations that increase the solubility of the isolated polypeptide or adaptor molecule.

In some embodiments, the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises one or more point mutations that increase the solubility of the isolated polypeptide or adaptor molecule, for example 1, 2, 3, 4, 5 or more point mutations that increase the solubility of the isolated polypeptide or adaptor molecule.

In some embodiments, the isolated polypeptide capable of binding to an adeno-associated virus (AAV) capsid or AAV adaptor molecule comprising an isolated polypeptide capable of binding to an adeno associated virus (AAV) capsid and a ligand comprises one or more point mutations that increase the yield of protein expression and/or purification of the isolated polypeptide or adaptor molecule in bacterial and/or mammalian systems, for example 1, 2, 3, 4, 5 or more point mutations that increase the yield of protein expression and/or purification of the isolated polypeptide or adaptor molecule in bacterial and/or mammalian systems.

In some embodiments, the isolated polypeptide capable of binding to an adeno-associated virus (AAV) capsid or AAV adaptor molecule comprising an isolated polypeptide capable of binding to an adeno associated virus (AAV) capsid and a ligand comprises a PKD2 domain of AAVR comprising one or more point mutations that increase the solubility of the isolated polypeptide or adaptor molecule, for example 1, 2, 3, 4, 5 or more point mutations that increase the solubility of the isolated polypeptide or adaptor molecule.

In some embodiments, the isolated polypeptide capable of binding to an adeno-associated virus (AAV) capsid or AAV adaptor molecule comprising an isolated polypeptide capable of binding to an adeno associated virus (AAV) capsid and a ligand comprises a PKD2 domain of AAVR comprising one or more point mutations that increase the yield of protein expression and/or purification of the isolated polypeptide or adaptor molecule in bacterial and/or mammalian systems, for example 1, 2, 3, 4, 5 or more point mutations that increase the yield of protein expression and/or purification of the isolated polypeptide or adaptor molecule in bacterial and/or mammalian systems.

A particular advantage of the present invention is that through the use of AAV-binding proteins as part of adaptor molecules, the tropism of AAVs can be altered essentially without loss of the packaging efficiency of recombinant AAV vectors into the capsid of the virus, in particular the infectivity for cells of low susceptibility can be increased several times, or the infectivity for cells of high susceptibility can be reduced several times. The present invention is therefore particularly suitable for an improved in vitro and in vivo transduction of particular cells, for example for somatic gene therapy.

In some embodiments, the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises one or more point mutations that increase the yield of protein expression and/or purification of the isolated polypeptide or adaptor molecule in bacterial and/or mammalian systems, for example 1, 2, 3, 4, 5 or more point mutations that increase the yield of protein expression and/or purification of the isolated polypeptide or adaptor molecule in bacterial and/or mammalian systems.

In some embodiments, the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a PKD2 domain of AAVR comprising one or more point mutations that increase the solubility of the isolated polypeptide or adaptor molecule, for example 1, 2, 3, 4, 5 or more point mutations that increase the solubility of the isolated polypeptide or adaptor molecule.

In some embodiments, the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a PKD2 domain of AAVR comprising one or more point mutations that increase the yield of protein expression and/or purification of the isolated polypeptide or adaptor molecule in bacterial and/or mammalian systems, for example 1, 2, 3, 4, 5 or more point mutations that increase the yield of protein expression and/or purification of the isolated polypeptide or adaptor molecule in bacterial and/or mammalian systems.

In some embodiments of the invention, the isolated polypeptide, AAV adaptor molecule or covalent binding virus adaptor molecule according to the invention may be bound to an AAV particle, AAV capsid, viral particle or viral capsid in the presence of a reducing agent. In some embodiments of the invention, the reducing agent is Tris(2-carboxyethyl)phosphine hydrochloride (TCEP). In some embodiments of the invention, the isolated polypeptide, AAV adaptor molecule or covalent binding virus adaptor molecule according to the invention may be partially oxidized prior to the coupling reaction. In some embodiments of the invention, the AAV particle, AAV capsid, viral particle or viral capsid according to the invention may be partially oxidized prior to the coupling reaction. In some embodiments of the invention, the isolated polypeptide, AAV adaptor molecule or covalent binding virus adaptor molecule according to the invention and the AAV particle, AAV capsid, viral particle or viral capsid according to the invention may be partially oxidized prior to the coupling reaction.

In some embodiments of the invention, the partially oxidised isolated polypeptide, AAV adaptor molecule or covalent binding virus adaptor molecule according to the invention may be bound to an AAV particle, AAV capsid, viral particle or viral capsid in the presence of a reducing agent. In some embodiments of the invention, the isolated polypeptide, AAV adaptor molecule or covalent binding virus adaptor molecule according to the invention may be bound to a partially oxidised AAV particle, AAV capsid, viral particle or viral capsid in the presence of a reducing agent. In some embodiments of the invention, the partially oxidised isolated polypeptide, AAV adaptor molecule or covalent binding virus adaptor molecule according to the invention may be bound to a partially oxidised AAV particle, AAV capsid, viral particle or viral capsid in the presence of a reducing agent. In some embodiments of the invention, the reducing agent is Tris(2-carboxyethyl)phosphine hydrochloride (TCEP).

In some embodiments of the invention, the “virus particle” or “AAV particle” may be a virus-like particle. In some embodiments of the invention, the “virus particle” or “AAV particle” may be a virus-like particle assembled in vitro following recombinant expression of capsid proteins. In some embodiments of the invention, the “virus particle” or “AAV particle” may be a virus-like particle assembled in vivo in a cellular expression system. In some embodiments of the invention, the cellular expression system may be a bacterial (e.g. E. coli), yeast (e.g. S. cerevisiae), plant, insect (e.g Trichoplusia) or mammalian (e.g. human) cellular expression system.

A particular advantage of the present invention is that through the use of binding proteins capable of covalently binding to viral capsids as part of adaptor molecules, the tropism of viruses can be altered essentially without loss of the packaging efficiency of recombinant vectors into the capsid of the virus, in particular the infectivity for cells of low susceptibility can be increased several times, or the infectivity for cells of high susceptibility can be reduced several times. The present invention is therefore particularly suitable for an improved in vitro and in vivo transduction of particular cells, for example for somatic gene therapy.

In any of the embodiments of the invention described herein, the isolated polypeptide capable of binding to a virus capsid or the viral adaptor molecule comprising an isolated polypeptide capable of binding to a viral capsid and a ligand (the “virus adaptor molecule”), may be capable of covalently binding to a virus capsid. Any of the statements above relating to non-covalent binding of an isolated polypeptide to a virus capsid apply mutatis mutandis to isolated polypeptides capable of covalently binding to a virus capsid. Specific embodiments relating to covalent binding of isolated polypeptides or viral adaptor molecules to virus capsids are described herein.

### BRIEF DESCRIPTION OF THE DRAWINGS

Embodiments of the present invention will now be described, by way of example only, with reference to the accompanying drawings, in which:

FIG. 1—Images showing expression of mScarlet gene in cells exposed to AAVs. HEK293FT expressing a synthetic membrane-anchored anti-sfGFP nanobody were treated as described. A—HEK293FT cells were infected by AAV2 wildtype carrying the mScarlet gene. B—HEK293FT cells were infected by AAV2 virus Arg585Ala Arg588Ala carrying the mScarlet gene. C—HEK293FT cells were exposed to the sfGFP-PKD2 adaptor protein, and then infected by AAV2 virus Arg585Ala Arg588Ala carrying the mScarlet gene. The data show that the sfGFP-PKD2 protein fusion greatly recovers the infectivity of AAV2 Arg585Ala Arg588Ala in cells expressing a partner receptor (see Example 6).

FIG. 2—SKBR3 cell line experiments showing infection with wildtype AAV2 with and without a DAPRin-PKD2 adaptor protein. A—SKBR3+AAV2 wt (−) DAPRin-PKD2, B—SKBR3+AAV2 wt (+) DAPRin-PKD2. The images show that the Her2-specific-DARPin-PKD2 fusion protein greatly enhances infectivity of AAV2 wildtype in SKBR3 cells.

FIG. 3—Experiments with HEK293FT cells showing infection with mScarlet-PKD2+AAV2 mix. The data show that the presence in solution of mScarlet-PKD2 fusion protein inhibits the infectivity of AAV2 wildtype in a concentration-dependent manner, suggesting that the PKD2 domain of the fusion protein can effectively interact with the virus and outcompete the cellular AAVR for binding, preventing the infection.

FIG. 4—Surface plasmon resonance (SPR) measurement of the binding kinetics of PKD2 fusion proteins to AAV2. The SPR signal confirms the interaction between the immobilized AAV2 and the fusion protein with measured affinity of around 6 μM.

FIG. 5—Surface plasmon resonance (SPR) measurement of the binding kinetics of PKD2 fusion proteins to AAV1. The SPR signal confirms the interaction between the immobilized AAV2 and the fusion protein with measured affinity of around 2 μM.

FIG. 6—Exemplary AAV receptor amino acid sequence. Derived from Uniprot entry Q81ZA0 and disclosed as SEQ ID NO:1. PKD 1 is residues 312-401, PKD 2 is residues 409-498, PKD 3 is residues 504-594, PKD 4 is residues 600-688 and PKD 5 is residues 694-785, all highlighted in bold and represented as SEQ ID NOs 2-6.

FIG. 7—Exemplary AAV1 cap gene amino acid sequence. Derived from NCBI entry NP_049542 and disclosed as SEQ ID NO:7.

FIG. 8—Exemplary AAV2 cap gene amino acid sequence. Derived from NCBI entry YP_680426 and disclosed as SEQ ID NO:8.

FIG. 9—Exemplary AAV3 cap gene amino acid sequence. Derived from NCBI entry NP_043941.1 and disclosed as SEQ ID NO:9.

FIG. 10—Exemplary AAV4 cap gene amino acid sequence. Derived from NCBI entry NP_044927 and disclosed as SEQ ID NO:10.

FIG. 11—Exemplary AAV5 cap gene amino acid sequence. Derived from NCBI entry YP_068409 and disclosed as SEQ ID NO:11.

FIG. 12—Exemplary AAV6 cap gene amino acid sequence. Derived from Uniprot entry 056137_9VIRU and disclosed as SEQ ID NO:12.

FIG. 13—Exemplary AAV7 cap gene amino acid sequence. Derived from NCBI entry YP_077178 and disclosed as SEQ ID NO:13.

FIG. 14—Exemplary AAV8 cap gene amino acid sequence. Derived from NCBI entry YP_077180 and disclosed as SEQ ID NO:14.

FIG. 15—Exemplary AAV9 cap gene amino acid sequence. Derived from Uniprot entry Q6JC40_9VIRU and disclosed as SEQ ID NO:15.

FIG. 16—Exemplary AAV10 cap gene amino acid sequence. Derived from NCBI entry AAT46337 and disclosed as SEQ ID NO:16.

FIG. 17—Exemplary AAV11 cap gene amino acid sequence. Derived from Uniprot entry Q5Y9B2_9VIRU and disclosed as SEQ ID NO:17.

FIG. 18—Exemplary AAV12 cap gene amino acid sequence. Derived from Uniprot entry A9RAI0 and disclosed as SEQ ID NO:18.

FIG. 19—Exemplary AAV13 cap gene amino acid sequence. Derived from Uniprot entry B5SUY7 and disclosed as SEQ ID NO:19.

FIG. 20—Formation of a disulfide bond between residue Cys590 of AAV1(Asp590Cys) mutant and Cys425 of sfGFP-PKD2(Ser425Cys) adaptor fusion protein. This figure shows the formation of three bands, migrating between the 150 kDa and 100 kDa markers, corresponding to the disulfide-bound sfGFP-PKD2+VP1, sfGFP-PKD2+VP2 and sfGFP-PKD2+VP3, respectively. These three bands can only be observed on a non-reducing gel (top) when AAV1 capsids with the mutations Asp590Cys were mixed with sfGFP-PKD2 with the mutation Ser425Cys, and are not observed otherwise. Furthermore, these bands are labile in the presence of reducing agents such as β-mercaptoethanol (PME, bottom gel)—see Example 6.

FIG. 21—Isolation of the covalently bound AAV1 in complex with an adaptor protein. The figure shows the composition of the AAV1-sfGFP CAR-V complex purified by iodixanol step gradient ultracentrifugation. Stably-bound heterodimers of the PKD2 mutant fusion protein and the viral capsid proteins VP1, VP2 and VP3 could be recovered from the 40% iodixanol fraction (bands above 100 kDa). This proves the successful formation of a covalently bound AAV1Cys< >sfGFP particle (see Example 2).

FIG. 22—Tropism of AAV1 covalently bound to an adaptor. The data shows that AAV1(Asp590Cys)< >sfGFP CAR-V has reduced tropism towards HEK293T cells that are not expressing the chimeric sfGFP receptor; but that infectivity is recovered when the target cells express the chimeric receptor. The data further show that HEK293T cells are highly susceptible to infection with AAV1(Asp590Cys) without the adaptor protein (see Example 3).

FIG. 23—Exemplary coxsackievirus and adenovirus receptor (CxAdR) cap gene amino acid sequence. Derived from Uniprot entry P78310 and disclosed as SEQ ID NO:20.

FIG. 24—Exemplary A20 antibody sequence. Derived from McCraw et al., Structure of adeno-associated virus-2 in complex with neutralizing monoclonal antibody, Volume 431, Issues 1-2, 15-30 Sep. 2012, Pages 40-49 and disclosed as SEQ ID NOs:21-28

FIG. 25—Exemplary CD46 sequence. Derived from Uniprot entry P15529 and disclosed as SEQ ID NO:29.

FIG. 26—Affinity of the PKD2(V480E) mutant for AAV1 capsids. Response-concentration plot (Y axis: in arbitrary units, X axis: molarity) for the affinity between AAV1 capsid and the PKD2 domains of AAVR, either wild type (indicated by squared, fitted by a solid line) or for the Val480Glu mutation (indicated by circles, fitted by a dashed line). The plot indicates that the Kd measured for the PKD2 wild type is 12.18 μM and for the PKD2(V480E) mutant is 6.13 μM, indicating that the mutant has higher affinity to the AAV1 capsid. Thus, we have discovered that the PKD2(V480E) variant has improved affinity for the AAV capsid, which makes it superior as an adapter protein.

FIG. 27—AdV-5 fibre knob coupling with CAR-derived Adaptor-protein. Reaction between the fibre knob protein from adenovirus-5 containing the mutation V441C and the adaptor protein derived from the human coxsackievirus-adenovirus receptor (CAR) containing the mutation V70C. The reaction was carried out in 20 mM Tris-HCl pH 8, 150 mM NaCl and 0.1 mM CuSO4. The final concentration of the knob protein (22 kDa) was 2 μM and the concentration of the adaptor protein (42 kDa) was 4 μM. The protein bands were resolved in a non-reducing 4-12% bis-tris gel. Lane 3 shows the formation of a band with molecular weight around 65 kDa, corresponding to the covalent adduct between the knob protein and the CAR-derived adaptor protein. We have therefore demonstrated that the use of adaptor proteins to retarget viruses can be applied to adenovirus using CAR-fusion proteins as adaptors.

FIG. 28—Negative-stain electron microscopy of AAV particles covalently bound to AAVR-derived adaptor protein. Negative-stain electron microscopy of AAV particles at a magnification of 52000×. A) AAV1(Asp590Cys) particles were purified by affinity chromatography (Cytiva AVB Sepharose resin) followed by iodixanol gradient ultracentrifugation, stained by phosphotungstic acid and imaged by electron microscopy. The figure reveals particles with a smooth contour. B) AAV1(Asp590Cys) particles were purified by affinity chromatography (Cytiva AVB Sepharose resin), then they were incubated with the sfGFP-PKD2(Ser425Cys) adaptor protein (3 μM, 20h at room temperature). The particles were then purified by iodixanol gradient ultracentrifugation, stained by phosphotungstic acid and imaged by electron microscopy. The figure reveals particles with a rough contour and larger than the native particles. C) Quantification of the diameter of the particles. The native particles in A) have average diameter of 26.0 nm, while the adaptor protein-coated particles in B) have an average diameter of 29.0 nm. This figure shows the complexes between AAV1(Asp590Cys) and an adaptor protein.

FIG. 29—AAV1^{Asp590Cys} coupling with PKD2-derived adaptor proteins. Coomassie-stained SDS PAGE showing that PKD2-derived fusion proteins containing a cysteine and encompassing various fusion partners can form covalent bond with AAV1^{Asp590Cys} The fusion partners used are: a single domain antibody against eGFP (a:eGFP sdAb); a DARPin evolved to bind Her2 (a:Her2 DARPin), a monomeric variant of super folder GFP (msfGFP) and the maltose-binding protein from E. coli (MBP). All the adaptor proteins were coupled with AAV1^{Asp590Cys} for 2 h at room temperature at a final concentration of 3 μM. The proteins were run on a non-reducing 4-12% bis-tris gradient gel. This figure shows that the identity of the fusion partner which composes the adaptor protein together with PKD2(Ser425Cys) can be customized in a modular fashion while maintaining the ability to form a covalent adduct. This implies that our technology can be used to retarget the AAVs towards many different receptors by changing the identity of the fusion partner.

Figure legend: * represents a capsid protein, † represents an adaptor protein, †† represents an adaptor protein dimer, *† represents a conjugate species between an adaptor protein and a viral protein.

FIG. 30—AAV1^{Asp590Cys} coupling with PKD2-derived adaptor proteins in 1 mM TCEP. Blot against the AAV1^{Asp590Cys} capsid proteins when the virus was in isolation (first lane after the marker) or incubated with 3 μM of an adaptor protein composed of a fusion partner as indicated in the figure, together with PKD2(Ser425Cys-Val480Glu). Incubations were carried out for 20 h at room temperature in presence of 1 mM TCEP. After incubation, the reactions were run on an 8% bis-tris non-reducing gel and run in MOPS buffer for 37 min at 200V. After the run, the gel was transferred on a PVDF membrane and immunoblotted using the primary mouse monoclonal 1 antibody against AAV VP1/2/3 (Progen, Cat. No. 690058) and an infrared IRDye® 8000W Goat anti-Mouse IgG Secondary Antibody (Li-COR, Cat. No. 926-32210). The figure shows that the covalently bound adducts between the adaptor protein and the viral capsid protein can be formed even in the presence of 1 mM of the reducing agent TCEP, indicating that the disulfide bond between the proteins is formed at an interface which is not fully solvent exposed. Notably, adducts can be formed between the viral protein and the adaptor both when the adaptor is expressed in bacterial cells (e.g. sfGFP and DARPins) and when the adaptor is expressed in mammalian cells (e.g. a:ICAM scFv).

FIG. 31—Infection of SKOV3 cells with AAV1 wild type or anti-EGFR-AAV1. Infection of SKOV3 cells by an AAV coated by an (anti:EGFR)DARPin-PKD2(Ser425Cys-Val480Glu) fusion protein shows that this cell line, which is normally poorly sensitive to AAV1 administered at high doses (1.5×107 vg/μL of medium), shows that this cell line is 3x more susceptible to infection by the coated virus. Infectivity was measured by the fraction of cells which expressed GFP (whose gene was delivered by the virus) after 72h of infection. Data plotted as mean of three replicates and analysed via two-tailed unpaired t-test. Error bars indicate standard deviation. Significance represented by ns: p>0.05, *=p<0.05, **=p<0.01, ***=p<0.001. This figure shows that re-targeting of AAV1(Asp590Cys) by means of an adaptor protein can substantially increase its infectivity.

FIG. 32—ADK1a neutralization—effect of anti-AAV1 mAbs on rAAV1 transgene expression. 0.5×10⁹ vg of recombinant AAV1 or 0.5×10⁹ vg of AAV1(Asp590Cys)< >(anti:EGFR)DARPin-PKD2(Ser425Cys-Val480Glu) CAR-V were incubated with serial dilution of the ADK1a neutralizing antibody (Progen, Cat. No. 610150) for 1 h at room temperature, then the mixture was used to infect HEK293T cells seeded in a 96-well plate. The fraction of infected cells, as measured by expression of the GFP reporter, was measured after 24 h and plotted as a function of the concentration of the antibody used. The plot shows that while recombinant AAV1 gets neutralized by the ADK1a antibody, the coated CAR-V particles maintain the same level of infectivity regardless of the concentration of neutralizing antibody and are hence resistant to neutralization.

FIG. 33—Infection of 293T, CHO and SKBR3 cell lines with coated AAV(Asp590Cys). Percentage of transduced cells 48-hours post-infection with coated and uncoated AAV1(Asp590Cys) viral particles at a final concentration of 2.0×10⁶vg per μL of cell medium. Coating of the virus was performed in the presence of 1 μM PKD2(Ser425Cys-Val480Glu) adaptor protein for 2 hours at room temperature. Quantification based on number of cells expressing the reporter delivered by the viruses assessed by laser scanning confocal microscopy. The x axis indicates the receptor targeted by the adaptor protein used to coat the virus. Data plotted as mean of two replicates. Infection of the SKBR3 cell line (C), which expresses high levels of HER2 and EGFR, indicates a higher infectivity by AAV1(Asp590Cys) viral particles coated with the corresponding PKD2(Ser425Cys-Val480Glu) adaptor protein. Coating AAV1(Asp590Cys) knocks down infectivity in CHO cells (B), which do not possess the corresponding receptors. This data show that coating AAV1(Asp590Cys) with adaptor proteins can enhance its specificity towards cell types expressing the receptor which interacts with the adaptor protein.

FIG. 34—Hepatic AAV transduction in TZM-bl xenograft model. Liver infectivity by AAV1(Asp590Cys) coated in sfGFP-PKD2(Ser425Cys-Val480Glu) and (anti-CD4)DARPin-PKD2(Ser425Cys-Val480Glu) relative to uncoated AAV1(Asp590Cys). Livers harvested 14 days after intravenous injection of C57BL/6Jax mice with coated and uncoated AAV1(Asp590Cys) particles at 3.2×10¹⁰vg/mouse. The mice hosted a tumor composed of TZM-bl cells. The data shows that coating of the virus with adaptor proteins abolishes infection of the liver, the main site of off-target infection for AAVs. Quantification based on three 50 μm sections of the liver for each mouse. Images acquired with a laser scanning confocal microscope. Data plotted as mean of three replicates.

FIG. 35—AAV5^{Q697C} coupling with PKD1^{S356C}-derived AP. Blot against the AAV5 capsid proteins on AAV5^{Gln697Cys} of the virus in isolation (first lane after the marker) or incubated with 10 μM of an adaptor protein (38.2 kDa) composed of superfolder GFP fused to PKD1(Ser356Cys). Incubations were carried out for 16 h at room. After incubation, the reactions were run on an 8% bis-tris non-reducing gel and run in MOPS buffer for 37 min at 200V. The gel was then transferred on a PVDF membrane and immunoblotted using the primary mouse monoclonal 1 antibody against AAV VP1/2/3 (Progen, Cat. No. 690058) and an infrared IRDye® 8000W Goat anti-Mouse IgG Secondary Antibody (Li-COR, Cat. No. 926-32210). The figure shows the covalently bound adducts between the adaptor protein and the viral capsid proteins, as indicated by the shift in molecular weight of the capsid proteins. Importantly, no appreciable bands corresponding to free VP proteins can be observed after incubation with the adaptor protein, indicating that the coupling reaches completion. This indicates that PKD1-derived adaptor proteins can be used to retarget AAV5^{Q697C}.

FIG. 36—AdV B3 Knob coupling DSG2-derived AP. Coomassie stained gels showing the covalent coupling between the adenovirus B3 fibre knob (residues 128-319, Uniprot ID P04501, strep-tagged, Asn192Cys mutant, 23.4 kDa) and an adaptor protein (AP) obtained by fusing superfolder GFP to desmoglein2 (residues 150-385, Ala174Cys mutant). The AP has molecular weight of 54.4 kDa. 3 μg of purified knob was incubated with 4 μg of purified adaptor protein and incubated for 18h in PBS at room temperature in a total reaction volume of 15 μL, in the absence (left) or presence (right) of 1 mM CaCl₂. The reactions were then run on a 4-12% bis-tris acrylamide gel with MES running buffer for 40 min at 200V in non-reducing conditions. The gel was then stained with Coomassie dye. The gel shows the formation of a covalent adduct between the fibre knob and the adaptor protein which is observed exclusively in the presence of calcium ions, indicating that the formation of the bond is dependent on the Ca2+-dependent interaction between the knob and desmoglein2. Hence, DSG2-derived adaptor proteins can be covalently coupled to adenovirus B3 and used to redirect its tropism.

FIG. 37—Exemplary Human Adenovirus B3 fibre protein sequence. Derived from Uniprot entry P04501 and disclosed as SEQ ID NO:30.

FIG. 38—Exemplary desmoglein 2 (DSG2) sequence. Derived from Uniprot entry Q14126 and disclosed as SEQ ID NO:31.

FIG. 39—Infection of 293T cells with coated AAV2(Gln589Cys). Percentage of transduced cells 48-hours post-infection with coated and uncoated AAV2(Gln589Cys) viral particles at a final concentration of 1.6×10⁶vg per μL of cell medium. Coating of the virus was performed in the presence of 3 μM PKD2(Ser425Cys-Val480Glu) adaptor protein for 24 hours at room temperature. Quantification based on number of cells expressing the reporter delivered by the viruses assessed by laser scanning microscopy. The x axis indicates the receptor targeted by the adaptor protein used to coat the virus. Data plotted as mean of two replicates. The virus is more infectious towards 293T cells when coated with adaptor proteins against HER2 and EGFR, which are expressed at the cell surface. Infectivity is knocked down when coated with GFP, for which there is no cell surface receptor present. The data shows that coating AAV2(Gln589Cys) with adaptor proteins can enhance its specificity towards cells expressing the receptor which interacts with the adaptor protein.

FIG. 40—AAV2(Gln589Cys) coupling with PKD2-derived adaptor proteins. Blot against the AAV1^{Asp590Cys} and AAV2^{Gln589Cys} capsid proteins with the virus in isolation (first and third lane after the marker, respectively) or incubated with 5 μM of superfolder GFP fused to PKD2(Ser425Cys-Val480Glu). Incubations were carried out for 22 h at room temperature. After incubation, the reactions were run on an 8% bis-tris non-reducing gel and run in MOPS buffer for 40 min at 200V. The gel was then transferred on a PVDF membrane and immunoblotted using the primary mouse monoclonal B1 antibody against AAV VP1/2/3 (Progen, Cat. No. 690058) and an infrared IRDye® 800CW Goat anti-Mouse IgG Secondary Antibody (Li-COR, Cat. No. 926-32210). The figure shows the covalently bound adducts between the adaptor protein and the viral capsid proteins, as indicated by the shift in molecular weight of the capsid proteins in lanes 2 and 5 after the marker. Importantly, the adduct bands are formed for both the AAV1 and AAV2 mutant viruses. This confirms that PKD2-derived adaptor proteins can be used to redirect the tropism of AAV1 and AAV2.

FIG. 41—Infection of SKBR3 cells with coated AAV5(Gln697Cys). Percentage of transduced cells 6 days post-infection with coated and uncoated AAV5(Gln697Cys) viral particles at a final concentration of 1.0×10⁷vg per μL of cell medium. Coating of the virus was performed in the presence of 0.5 μM PKD1(Ser356Cys) adaptor protein for 24 hours at room temperature. Quantification based on number of cells expressing the reporter delivered by the viruses assessed by laser scanning microscopy. The x axis indicates the receptor targeted by the adaptor protein used to coat the virus. Data plotted as mean of two replicates. The virus is more infectious towards SKBR3 cells when coated with an adaptor protein against HER2, which is highly expressed at the cell surface. Infectivity is knocked down when coated with GFP, for which there is no cell surface receptor present. The data shows that coating AAV5(Gln697Cys) with adaptor proteins can permit retargeting by enhancing its specificity towards cells expressing the corresponding receptor.

### DETAILED DESCRIPTION OF THE INVENTION

In order for the present invention to be more readily understood, certain terms are first defined below. Additional definitions for the following terms and other terms are set forth throughout the specification.

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%), 6%), 5%, 4%, 3%, 2%, 1%), or less in either direction (greater than or less than) of the stated reference value unless otherwise stated or otherwise evident from the context (except where such number would exceed 100% of a possible value).

As used herein, the term “amelioration” means the prevention, reduction or palliation of a state, or improvement of the state of a subject. Amelioration includes, but does not require, complete recovery or complete prevention of a disease condition.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogues known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “ligand” refers to a molecule capable of mediating an interaction with a biological molecule, for example a cell surface receptor. A ligand may comprise a portion of a protein or polypeptide or a full-length sequence of a protein or polypeptide. A ligand may also comprise any other molecule capable of mediating an interaction with a biological molecule, for example a cell surface receptor, such as a suitable small molecule. In some embodiments of the invention, the ligand mediates a biological interaction with a target cell. In some embodiments of the invention, the ligand mediates a different biological interaction to the interaction between the isolated polypeptide or adaptor molecule and the virus particle or virus capsid.

As used herein, the term “full-length” of a protein or polypeptide refers to the entire canonical protein including substantially all of the amino acids indicated in the sequence. As used herein, the term “portion” of a protein or polypeptide refers to a characteristic part of the full-length protein capable of achieving substantially the same function as the full-length protein (e.g. binding of a target). The term “portion” of a protein or polypeptide may refer to part of a protein or polypeptide comprising at least 50%, 60%, 70%, 80%, 90%, 95%, 96%, 97%, 98% or 99% of the amino acids of the full-length protein or polypeptide. As used herein, a “portion” of a protein or polypeptide may comprise at least 100, 200, 300, 400, 500 or more amino acids.

As used herein, the term “conservative” changes or “conservative amino acid changes”, refers to where a substituted amino acid has similar structural or chemical properties. With reference to a protein or polypeptide, a conservative change is an amino acid change that does not affect at least one function of the protein or polypeptide (for example, an amino acid change that does not affect the function of a viral capsid. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As used herein, the term “non-conservative” changes or “non-conservative amino acid changes” refers to replacement of an amino acid with another amino acid having different structural or chemical properties, such as glycine with a tryptophan. With reference to a protein or polypeptide, a non-conservative change is an amino acid change that affects at least one function of the protein or polypeptide (for example, an amino acid change that affects the function of a viral capsid. Guidance in determining which and how many amino acid residues may be substituted, inserted or deleted without affecting biological activity may be found using computer programs well known in the art, for example, DNAStar software. Variants can be tested in functional assays. Certain variants have less than 10%, and preferably less than 5%, and still more preferably less than 2% or less than 1% substituted amino acids. As used herein, the term “point mutation” refers to a single amino acid substitution.

As used herein, the term “insertion” refers to the introduction of one or more contiguous heterologous amino acid residues (i.e. amino acids which do not appear in the corresponding position in the wildtype amino acid sequence). An insertion may be 1, 2, 3, 4, 5, 6, 7, 8, 9, 10 or more amino acids in length. When referring to viral capsids not having an insertion, preferably the viral capsids do not comprise insertions of more than 5 amino acids in length. Means of introducing an insertion will be known to skilled person, for example via genetic or chemical means.

As used herein, the term “deletion” refers to the removal of one or more contiguous amino acid residues from an amino acid sequence. A deletion be 1, 2, 3, 4, 5, 6, 7, 8, 9, 10 or more amino acids in length. When referring to viral capsids not having deletion, preferably the viral capsids do not comprise deletions of more than 5 amino acids in length. Means of introducing a deletion will be known to skilled person, for example via genetic or chemical means.

As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In a preferred embodiment of the invention the subject is a human.

As used herein, the terms “target cell” or “target tissue” refers to any cell, tissue, or organism. In some embodiments, the target cell or target tissue is the cell or tissue involved in a pathological condition which is susceptible to treatment via gene therapy via viral vectors.

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g. transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

In some embodiments of the invention, the methods of treatment comprise administration of a composition or viral particle described herein in a therapeutically effective amount. In some embodiments of the invention, the compositions described for use herein are administered in a therapeutically effective amount.

As used herein, the term “treatment” (also “treat” or “treating”) refers to any administration of a therapeutic agent according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition. In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “antibody” includes immunoglobulin molecules comprising four polypeptide chains, two heavy (H) chains and two light (L) chains inter-connected by disulfide bonds. Each heavy chain comprises a heavy chain variable domain (VH) and a heavy chain constant region (CH). The heavy chain constant region comprises at least three domains, CH 1, CH 2, CH 3 and optionally CH 4. Each light chain comprises a light chain variable domain (CH) and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. Typical tetrameric antibody structures comprise two identical antigen-binding domains, each of which formed by association of the VH and VL domains, and each of which together with respective CH and CL domains form the antibody Fv region. Single domain antibodies comprise a single antigen-binding domain, e.g., a VH or a VL. The antigen-binding domain of an antibody, e.g., the part of an antibody that recognizes and binds to the first member of a specific binding pair of an antigen, is also referred to as a “paratope.” It is a small region (of 5 to 10 amino acids) of an antibody's Fv region, part of the fragment antigen-binding (Fab region), and may contains parts of the antibody's heavy and/or light chains. A paratope specifically binds a first member of a specific binding pair when the paratope binds the first member of a specific binding pair with a high affinity. The term “high affinity” antibody refers to an antibody that has a KD with respect to its target first member of a specific binding pair about of 10-9 M or lower (e.g., about 1×10-9 M, 1×10-10 M, 1×10-11 M, or about 1×10-12 M). In one embodiment, KD is measured by surface plasmon resonance, e.g., BIACORE™; in another embodiment, KD is measured by ELISA.

The phrase “complementarity determining region,” or the term “CDR,” includes an amino acid sequence encoded by a nucleic acid sequence of an organism's immunoglobulin genes that normally (i.e., in a wildtype animal) appears between two framework regions in a variable region of a light or a heavy chain of an immunoglobulin molecule (e.g., an antibody or a T cell receptor). A CDR can be encoded by, for example, a germ line sequence or a rearranged or unrearranged sequence, and, for example, by a naive or a mature B cell or a T cell. A CDR can be somatically mutated (e.g., vary from a sequence encoded in an animal's germ line), humanized, and/or modified with amino acid substitutions, insertions, or deletions. In some circumstances (e.g., for a CDR3), CDRs can be encoded by two or more sequences (e.g., germ line sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH 1 domain, a hinge, a CH 2 domain, and a CH 3 domain. A functional fragment of a heavy chain includes a fragment that is capable of specifically recognizing an first member of a specific binding pair (e.g., recognizing the first member of a specific binding pair with a KD in the micromolar, nanomolar, or picomolar range), that is capable of expressing and secreting from a cell, and that comprises at least one CDR. Heavy chain variable domains are encoded by variable region nucleotide sequence, which generally comprises VH, DH, and JH segments derived from a repertoire of VH, DH, and JH segments present in the germline.

The phrase “light chain” includes an immunoglobulin light chain sequence from any organism, and unless otherwise specified includes human K and A light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises VL and JL, segments, derived from a repertoire of V and J segments present in the germ line. Sequences, locations and nomenclature for V and J light chain segments for various organisms can be found in IMGT database, www.imgt.org. Light chains include those, e.g., that do not selectively bind either a first or a second first member of a specific binding pair selectively bound by the first member of a specific binding pair-binding protein in which they appear. Light chains also include those that bind and recognize, or assist the heavy chain or another light chain with binding and recognizing, one or more first member of a specific binding pairs selectively bound by the first member of a specific binding pair-binding protein in which they appear. Common or universal light chains include those derived from a human Vκ1-39Jκ gene or a human Vκ3-20Kκ gene, and include somatically mutated (e.g., affinity matured) versions of the same. Exemplary human VL segments include a human Vκ1-39 gene segment, a human Vκ3-20 gene segment, a human VA1-40 gene segment, a human Vλ1-44 gene segment, a human Vλ2-8 gene segment, a human Vλ2-14 gene segment, and human Vλ3-21 gene segment, and include somatically mutated (e.g., affinity matured) versions of the same. Light chains can be made that comprise a variable domain from one organism (e.g., human or rodent, e.g., rat or mouse; or bird, e.g., chicken) and a constant region from the same or a different organism (e.g., human or rodent, e.g., rat or mouse; or bird, e.g., chicken).

The term “capsid protein” includes a protein that is part of the capsid of the virus. For adeno-associated viruses, the capsid proteins are generally referred to as VP1, VP2 and/or VP3, and are encoded by the single cap gene. For AAV, the three AAV capsid proteins are produced in an overlapping fashion from the cap open reading frame (ORF) via alternative mRNA splicing and/or alternative translational start codon usage, although all three proteins use a common stop codon.

The term “wildtype”, as used herein, includes an entity having a structure and/or activity as found in nature in a “normal” (as contrasted with mutant, diseased, altered, etc.) state or context. Those of ordinary skill in the art will appreciate that wildtype viral vectors, e.g., wildtype capsid proteins, may be used as reference viral vector in comparative studies with modified capsids. Generally, a reference viral capsid protein/capsid/vector are identical to the test viral capsid protein/capsid/vector but for the change for which the effect is to be tested. When used in reference to polynucleotides or polypeptides, “wildtype” refers to the native (unmodified) form of the polynucleotide or polypeptide as found within, or expressed by, the wildtype organism.

As used herein, the term “covalent”, “covalent bond” or “covalently bound” refers to a chemical bond that involves the sharing of electron pairs between atoms. These electron pairs are known as shared pairs or bonding pairs, and the stable balance of attractive and repulsive forces between atoms forms a covalent bond. For many molecules, the sharing of electrons allows each atom to attain the equivalent of a full valence shell, corresponding to a stable electronic configuration. In a preferred embodiment of the invention, the covalent bond is a disulfide (SS-bond) and can be derived by the coupling of two thiol groups. Covalent bonds between thiol groups in two cysteine residues are an important component of the secondary and tertiary structure of proteins. Covalent bonds can provide a stronger linkage between one or more biological molecules than other forms of bonding such as ionic bonding or electromagnetic intermolecular forces.

As used herein, the term “virus adaptor molecule” refers to a protein, fusion protein, or conjugated compound comprising a polypeptide capable of binding to a virus capsid and a ligand. The term “virus adaptor” may also be used to refer to such compounds. In some embodiments, the term “virus adaptor molecule” refers to a protein, fusion protein, or conjugated compound comprising a polypeptide comprising one or more cysteine residues capable of covalently binding to a viral capsid and a ligand. The terms “virus adaptor” or “covalent virus adaptor” may also be used to refer to such compounds. As used herein, the term “AAV adaptor molecule” refers to a protein, fusion protein, or conjugated compound comprising a polypeptide capable of binding to an adeno associated virus (AAV) capsid and a ligand. The term “AAV adaptor” may also be used to refer to such compounds.

### Gene Therapy

In some embodiments, recombinant viral vectors as described herein comprise a virus particle bound to an isolated polypeptide capable of binding to a viral capsid or a virus adaptor molecule comprising an isolated polypeptide capable of binding to a viral capsid and a ligand, wherein the virus particle encapsulates a nucleotide of interest. In some embodiments, the nucleotide of interest is under the control of a promoter selected from the group consisting of a viral promoter, a bacterial promoter, a mammalian promoter, an avian promoter, a fish promoter, an insect promoter, and any combination thereof. In some embodiments, the nucleotide of interest is under the control of a non-human promoter. In some embodiments, the promoter is a cytomegalovirus (CMV) promoter. In some embodiments, the promoter is an EF1α promoter. In some embodiments, the promoter is a CAGG promoter. In some embodiments, the promoter is a Ubiquitin C (UbC) promoter.

In some embodiments, recombinant viral vectors as described herein comprise a virus particle covalently bound to an isolated polypeptide comprising one or more heterologous cysteine residues capable of covalently binding to a viral capsid or a virus adaptor molecule comprising an isolated polypeptide comprising one or more heterologous cysteine residues capable of covalently binding to a viral capsid and a ligand, wherein the virus particle encapsulates a nucleotide of interest. In some embodiments, the nucleotide of interest is under the control of a promoter selected from the group consisting of a viral promoter, a bacterial promoter, a mammalian promoter, an avian promoter, a fish promoter, an insect promoter, and any combination thereof. In some embodiments, the nucleotide of interest is under the control of a non-human promoter. In some embodiments, the promoter is a cytomegalovirus (CMV) promoter. In some embodiments, the promoter is an EF1a promoter. In some embodiments, the promoter is a CAGG promoter. In some embodiments, the promoter is a Ubiquitin C (UbC) promoter.

Generally, a nucleotide or a gene of interest may be one or more genes, which may encode a detectable marker, e.g., reporter, or a therapeutic polypeptide. In some embodiments, the nucleotide of interest is a reporter gene. In some embodiments, the nucleotide or gene of interest is a reporter gene that encodes a detectable marker selected from the group consisting of green fluorescent protein, luciferase, p-galactosidase, etc. In some embodiments, the detectable marker is green fluorescent protein. In other embodiments, the nucleotide of interest is selected from the group consisting of a suicide gene, a nucleotide encoding an antibody or fragment thereof, a nucleotide encoding a CRISPR/Cas system or portion(s) thereof, a nucleotide encoding antisense RNA, a nucleotide encoding siRNA, a secreted enzyme, a gene encoding a therapeutic protein, etc. In one embodiment, the nucleotide of interest encodes a multidomain therapeutic, e.g., a protein that comprises at least two domains providing two distinct functions.

Compositions described herein may comprise a viral vector that comprises a recombinant viral particle as described herein, e.g., comprises a viral particle bound to an isolated polypeptide capable of binding to a viral capsid or a virus adaptor molecule comprising an isolated polypeptide capable of binding to a viral capsid and a ligand, wherein the virus particle encapsulates a nucleotide or gene of interest.

Compositions described herein may comprise a viral vector that comprises a recombinant viral particle as described herein, e.g., comprises a viral particle covalently bound to an isolated polypeptide comprising one or more heterologous cysteine residues capable of covalently binding to a viral capsid or a virus adaptor molecule comprising an isolated polypeptide comprising one or more heterologous cysteine residues capable of covalently binding to a viral capsid and a ligand, wherein the virus particle encapsulates a nucleotide or gene of interest.

Also described herein are methods of making and using the recombinant viral capsid proteins, viral vectors comprising same, compositions, etc. In some embodiments, a method of redirecting a virus, e.g., an adenovirus, adeno-associated virus, etc.; delivering diagnostic/therapeutic cargo to a target cell, etc. comprises contacting a target cell (which may be in vitro or in vivo, e.g., in a human) with a recombinant viral vector comprising a recombinant viral capsid protein as described herein. Such methods may include as a first step producing a viral vector, e.g., culturing a packaging cell in conditions sufficient for the production of viral vectors, wherein the packaging cell comprises a plasmid encoding an isolated polypeptide capable of binding to a viral capsid or a virus adaptor molecule comprising an isolated polypeptide capable of binding to a viral capsid and a ligand. Such methods may include as a first step producing a viral vector, e.g., culturing a packaging cell in conditions sufficient for the production of viral vectors, wherein the packaging cell comprises a plasmid encoding an isolated polypeptide comprising one or more heterologous cysteine residues capable of covalently binding to a viral capsid or a virus adaptor molecule comprising an isolated polypeptide comprising one or more heterologous cysteine residues capable of covalently binding to a viral capsid and a ligand.

In some embodiments, the target cell is a (human) liver cell and the (mosaic) recombinant viral vector comprises a targeting ligand that specifically binds asialoglycoprotein receptor, e.g., (h)ASGR1. In some embodiments, the target cell is a (human) neuronal cell, and the (mosaic) recombinant viral vector comprises a targeting ligand that specifically binds GABA, transferrin receptor, etc. In some embodiments, the target cell is a (human) T cell, and the (mosaic) recombinant viral vector comprises a targeting ligand that specifically binds CD3, e.g., CD3E. In some embodiments, the target cell is a (human) hematopoietic stem cell, and the (mosaic) recombinant viral vector comprises a targeting ligand that specifically binds CD34. In some embodiments, the target cell is a (human) kidney cell. In some embodiments, the target cell a (human) muscle cell, and the (mosaic) recombinant viral vector comprises a targeting ligand that specifically binds an integrin. In some embodiments, the target cell is a (human) cancerous cell, and the (mosaic) recombinant viral vector comprises a targeting ligand that specifically binds a tumor associated antigen, e.g., E6 and E7, Her2, etc. In some embodiments, the targeting ligand binds human glucagon receptor (hGCGR).

The genetic cargo capacity of the viral particles provided herein (e.g. of the nucleotide of interest) can vary. The genetic cargo capacity of the virus particles can be, or can be about, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, 10.0 kb, or a number or a range between any two of these values. The genetic cargo capacity of the virus particles can be at least, or can be at most, 5.0 kb, 5.1 kb, 5.2 kb, 5.3 kb, 5.4 kb, 5.5 kb, 5.6 kb, 5.7 kb, 5.8 kb, 5.9 kb, 6.0 kb, 6.1 kb, 6.2 kb, 6.3 kb, 6.4 kb, 6.5 kb, 6.6 kb, 6.7 kb, 6.8 kb, 6.9 kb, 7.0 kb, 7.1 kb, 7.2 kb, 7.3 kb, 7.4 kb, 7.5 kb, 7.6 kb, 7.7 kb, 7.8 kb, 7.9 kb, 8.0 kb, 8.1 kb, 8.2 kb, 8.3 kb, 8.4 kb, 8.5 kb, 8.6 kb, 8.7 kb, 8.8 kb, 8.9 kb, 9.0 kb, 9.1 kb, 9.2 kb, 9.3 kb, 9.4 kb, 9.5 kb, 9.6 kb, 9.7 kb, 9.8 kb, 9.9 kb, or 10.0 kb. The genetic cargo capacity can be: (i) the maximum length of a single-stranded DNA molecule that the virus particle is capable of protecting from DNAse I digestion; and/or (ii) the maximum length of a double-stranded DNA molecule that the virus particle is capable of protecting from DNAse I digestion. The single-stranded DNA molecule can be capable of self-hybridizing to form a double-stranded region. The single-stranded DNA molecule can comprise a self-complementary AAV (scAAV) vector.

### Adeno-Associated Viruses (AAVs)

Adeno-associated viruses (AAVs) are small viruses that infect humans and some other primate species. They belong to the genus Dependoparvovirus, which in turn belongs to the family Parvoviridae. They are small (20 nm) replication-defective, nonenveloped viruses and have linear single-stranded DNA (ssDNA) genome of approximately 4.8 kilobases (kb).

AAVs are not currently known to cause disease. The viruses cause a very mild immune response. Several additional features make AAVs an attractive candidate for creating viral vectors for gene therapy, and for the creation of isogenic human disease models. Gene therapy vectors using AAV can infect both dividing and quiescent cells and persist in an extrachromosomal state without integrating into the genome of the host cell, although in the native virus integration of virally carried genes into the host genome does occur.

The AAV genome is built of single-stranded deoxyribonucleic acid (ssDNA), either positive- or negative-sensed, which is about 4.7 kilobase long. The genome comprises inverted terminal repeat (ITR) sequences at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact to form a capsid with icosahedral symmetry.

The inverted terminal repeat (ITR) sequences comprise 145 bases each. They are named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19th chromosome in humans) and rescue from it, as well as for efficient encapsidation of the AAV DNA combined with generation of a fully assembled, deoxyribonuclease-resistant AAV particles.

On the “left side” of the genome there are two promoters called p5 and p19, from which two overlapping messenger ribonucleic acids (mRNAs) of different length can be produced. Each of these contains an intron which can be either spliced out or not. Given these possibilities, four mRNAs, and consequently four Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter (mentioned below), but downregulate both p5 and p19 promoters.

The right side of a positive-sensed AAV genome encodes overlapping sequences of three capsid proteins, VP1, VP2 and VP3, which start from one promoter, designated p40. The molecular weights of these proteins are 87, 72 and 62 kilodaltons, respectively. The AAV capsid is composed of a mixture of VP1, VP2, and VP3 totalling 60 monomers arranged in icosahedral symmetry in a ratio of 1:1:10, with an estimated size of 3.9 megadaltons.

The cap gene produces an additional, non-structural protein called the Assembly-Activating Protein (AAP). This protein is produced from ORF2 and is essential for the capsid-assembly process.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is spliced out, resulting in a reduced overall level of VP1 protein synthesis. The first AUG codon that remains in the major splice is the initiation codon for VP3 protein. However, upstream of that codon in the same open reading frame lies an ACG sequence which is surrounded by an optimal Kozak context.

Since the bigger intron is preferably spliced out, and since in the major splice the ACG codon is a much weaker translation initiation signal, the ratio at which the AAV structural proteins are synthesized in vivo is about 1:1:10, which is the same as in the mature virus particle. The unique fragment at the N terminus of VP1 protein was shown to possess the phospholipase A2 (PLA2) activity, which is probably required for the releasing of AAV particles from late endosomes.

AAV is highly prevalent in humans and other primates and several serotypes have been isolated from various tissue samples. Serotypes 2, 3, 5, and 6 were discovered in human cells, AAV serotypes 1, 4, and 7-11 in nonhuman primate samples. AAV capsid proteins contain 12 hypervariable surface regions, with most variability occurring in the threefold proximal peaks, but the parvovirus genome in general presents highly conserved replication and structural genes across serotypes. All of the known serotypes can infect cells from multiple diverse tissue types. Tissue specificity is determined by the capsid serotype and pseudotyping of AAV vectors to alter their tropism range will likely be important to their use in therapy.

Serotype 2 (AAV2) has been the most extensively examined so far. AAV2 presents natural tropism towards skeletal muscles, neurons, vascular smooth muscle cells and hepatocytes. Three cell receptors have been described for AAV2: heparan sulfate proteoglycan (HSPG), aVβ5 integrin and fibroblast growth factor receptor 1 (FGFR-1). The first functions as a primary receptor, while the latter two have a co-receptor activity and enable AAV to enter the cell by receptor-mediated endocytosis. HSPG functions as the primary receptor, though its abundance in the extracellular matrix can scavenge AAV particles and impair the infection efficiency.

Studies have shown that serotype 2 of the virus (AAV2) cancer cells without harming healthy ones. “Our results suggest that adeno-associated virus type 2, which infects the majority of the population but has no known ill effects, kills multiple types of cancer cells yet has no effect on healthy cells,” said Craig Meyers,[69] a professor of immunology and microbiology at the Penn State College of Medicine in Pennsylvania in 2005.[70] This could lead to a new anti-cancer agent.

Other serotypes. Although AAV2 is the most popular serotype in various AAV-based research, it has been shown that other serotypes can be more effective as gene delivery vectors. For instance, AAV6 appears much better in infecting airway epithelial cells, AAV7 presents very high transduction rate of murine skeletal muscle cells (similar to AAV1 and AAV5), AAV8 is superb in transducing hepatocytes and AAV1 and 5 were shown to be very efficient in gene delivery to vascular endothelial cells. In the brain, most AAV serotypes show neuronal tropism, while AAV5 also transduces astrocytes. AAV6, a hybrid of AAV1 and AAV2, also shows lower immunogenicity than AAV2.

Serotypes can differ with the respect to the receptors they are bound to. For example, AAV4 and AAV5 transduction can be inhibited by soluble sialic acids (of different form for each of these serotypes), and AAV5 was shown to enter cells via the platelet-derived growth factor receptor.

There have been many efforts to engineer and improve new AAV variants for both clinical and research purposes. Such modifications include new tropisms to target specific tissues, and modified surface residues to evade detection by the immune system. Beyond opting for particular strains of recombinant AAV (rAAV) to target particular cells, researchers have also explored AAV pseudotyping, the practice of creating hybrids of certain AAV strains to approach an even more refined target. The hybrid is created by taking a capsid from one strain and the genome from another strain. For example, research involving AAV2/5, a hybrid with the genome of AAV2 and the capsid of AAV5, was able to achieve more accuracy and range in brain cells than AAV2 would be able to achieve unhybridized. Researchers have continued to experiment with pseudotyping by creating strains with hybrid capsids. AAV-DJ has a hybrid capsid from eight different strains of AAV; as such, it can infect different cells throughout many areas of the body, a property which a single strain of AAV with a limited tropism would not have. Other efforts to engineer and improve new AAV variants have involved the ancestral reconstruction of virus variants to generate new vectors with enhanced properties for clinical applications and the study of AAV biology.

### Chimeric Antigen Receptor Virus (CAR-V)

As used herein, Chimeric Antigen Receptor Virus, or CAR-V, is a viral particle whose tropism has been altered by means of the surface display of a synthetic protein (the chimeric antigen receptor) capable of binding to a cell surface ligand or antigen. In the exemplifications provided, a CAR-V can be obtained from AAV1 Asp590Cys virions by coupling overnight in a 500 μL reaction at room temperature, 0.1-1 pg of capsids with a synthetic receptor, in the form of the sfGFP-PKD2 adaptor molecule at a concentration of 15 μM, capable of binding to a synthetic surface antigen, in the form of an engineered surface expressed anti-sfGFP nanobody.

### Virus-Like Particles (VLPs)

Virus-like particles (VLPs) are molecules that resemble viruses, but are non-infectious because they contain no viral genetic material. They can be naturally occurring or synthesized through the individual expression of viral structural proteins, which can then self-assemble into a virus-like structure (Zeltins, Molecular Biotechnology volume 53, pages 92-107 (2013)). Combinations of structural capsid proteins from different viruses can be used to create recombinant VLPs. Both in-vivo assembly (i.e., assembly inside a cell such as an E. coli bacterium via recombinant co-expression of multiple proteins) and in-vitro assembly (i.e., protein self-assembly in a reaction vessel using stoichiometric quantities of previously purified proteins) can be used to form virus-like particles.

VLPs have been produced from components of a wide variety of virus families including Parvoviridae (e.g. adeno-associated virus), Retroviridae (e.g. HIV), Flaviviridae (e.g. Hepatitis C virus), Paramyxoviridae (e.g. Nipah) and bacteriophages (e.g. QR, Aβ205). VLPs can be produced in multiple cell culture systems including bacteria, mammalian cell lines, insect cell lines, yeast and plant cells.

VLPs can also refer to structures produced by some LTR retrotransposons in nature. These are defective, immature virions, sometimes containing genetic material, that are generally non-infective due to the lack of a functional viral envelope.

### Pharmaceutical Compositions of the Agent

Other aspects of the present invention also relate to a medicinal product or a diagnostic aid comprising an isolated polypeptide, an AAV adaptor molecule, a virus adaptor molecule, a virus particle, a virus-like particle, a nucleic acid or a cell according to the invention and, where appropriate, suitable excipients and additives, such as, for example, a physiological saline solution, stabilizers or proteinase inhibitors.

### Kits

In some embodiments of the invention, an agent (e.g. an isolated polypeptide, an AAV adaptor molecule, a virus adaptor molecule, a virus particle, a virus-like particle, a nucleic acid, a cell or a pharmaceutical composition) described herein can be provided in a kit. In some instances, the kit includes (a) a container that contains an agent described herein and, optionally (b) informational material. The informational material can be descriptive, instructional, marketing or other material that relates to the methods described herein and/or the use of an agent, e.g., for therapeutic benefit.

The informational material of the kits is not limited in its form. In some instances, the informational material can include information about production of a therapeutic agent, molecular weight of a therapeutic agent, concentration, date of expiration, batch or production site information, and so forth. In other situations, the informational material relates to methods of administering a therapeutic agent, e.g., in a suitable amount, manner, or mode of administration (e.g., a dose, dosage form, or mode of administration described herein).

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a therapeutic agent therein and/or their use in the methods described herein. The informational material can also be provided in any combination of formats.

In addition to a therapeutic agent, the kit can include other ingredients, such as a solvent or buffer, a stabilizer, or a preservative. The kit can also include further agents, e.g., a second or third agent, e.g., other therapeutic agents. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a therapeutic agent or other agents. In some cases, the kit contains separate containers, dividers or compartments for a therapeutic agent and informational material. For example, a therapeutic agent can be contained in a bottle, vial, or syringe, and the informational material can be contained in a plastic sleeve or packet. In other situations, the separate elements of the kit are contained within a single, undivided container. For example, a therapeutic agent can be contained in a bottle, vial or syringe that has attached thereto the informational material in the form of a label. In some cases, the kit can include a plurality (e.g., a pack) of individual containers, each containing one or more unit dosage forms (e.g., a dosage form described herein) of a therapeutic agent. The containers can include a unit dosage, e.g., a unit that includes a therapeutic agent. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of a therapeutic agent, e.g., a syringe or other suitable delivery device. The device can be provided preloaded with a therapeutic agent, e.g., in a unit dose, or can be empty, but suitable for loading.

### Combination Therapies

In some embodiments, the invention features a composition (e.g., one or more compositions, formulations or dosage formulations) or a pharmaceutical combination, comprising an isolated polypeptide capable of binding to a virus capsid or a viral adaptor molecule comprising an isolated polypeptide capable of binding to a viral capsid and a ligand according to the invention and a second therapeutic agent. In some embodiments, the second therapeutic agent is chosen from one or more agents selected from the list consisting of analgesics, anesthetics, antibacterials, anticonvulsants, antidementia agents, antidepressants, antidotes, antiemetics, antifungals, antigout agents, anti-inflammatory agents, antimigraine agents, antimyasthenic agents, antimycobacterials, antineoplastics, antiparasitics, antiparkinson agents, antipsychotics, antispasticity agents, antivirals, anxiolytics, bipolar agents, blood glucose regulators, cardiovascular agents, central nervous system agents, dental and oral agents, dermatological agents, enzyme replacements/modifiers, gastrointestinal agents, genitourinary agents, hormonal agents, inflammatory bowel disease agents, metabolic bone disease agents, ophthalmic agents, otic agents, respiratory tract agents, sedatives/hypnotics, skeletal muscle relaxants, therapeutic nutrients/minerals and electrolytes.

In some embodiments, the composition comprises a pharmaceutically acceptable carrier. In some embodiments, the isolated polypeptide capable of binding to a virus capsid or a viral adaptor molecule comprising an isolated polypeptide capable of binding to a virus capsid and a ligand, and the second agent can be present in a single composition or as two or more different compositions. The isolated polypeptide capable of binding to a virus capsid or a virus adaptor molecule comprising an isolated polypeptide capable of binding to a virus capsid and a ligand, and the second agent can be administered via the same administration route or via different administration routes. The isolated polypeptide capable of binding to a virus capsid or a virus adaptor molecule comprising an isolated polypeptide capable of binding to a virus capsid and a ligand, and the second agent can be administered simultaneously or sequentially. In some embodiments, the pharmaceutical combination comprises the isolated polypeptide capable of binding to a virus capsid or a virus adaptor molecule comprising an isolated polypeptide capable of binding to a virus capsid and a ligand, and the second agent separately or together.

### Routes of Administration

The agent or pharmaceutical composition can be administered by different routes including orally, parenterally, sublingually, transdermally, rectally, transmucosally, topically, via inhalation, via buccal administration, intrapleurally, intravenously, intraarterially, intraperitoneally, subcutaneously, intramuscularly, intranasal intrathecally, and/or intraarticularly, or combinations thereof. In some embodiments the agent or pharmaceutical composition is administered orally.

### Transduction Efficiencies

In some embodiments, a virus particle bound to an isolated polypeptide capable of binding to a viral capsid of the invention exhibits at least 10% decrease in transduction efficiency compared to an appropriate control wildtype virus particle. In some embodiments, a virus particle bound to an isolated polypeptide capable of binding to a viral capsid of the invention exhibits at least 20% decrease in transduction efficiency compared to an appropriate control wildtype virus particle. In some embodiments, a virus particle bound to an isolated polypeptide capable of binding to a viral capsid of the invention exhibits at least 30% decrease in transduction efficiency compared to an appropriate control wildtype virus particle. In some embodiments, a virus particle bound to an isolated polypeptide capable of binding to a viral capsid of the invention exhibits at least 40% decrease in transduction efficiency compared to an appropriate control wildtype virus particle. In some embodiments, a virus particle bound to an isolated polypeptide capable of binding to a viral capsid of the invention exhibits at least 50% decrease in transduction efficiency compared to an appropriate control wildtype virus particle. In some embodiments, a virus particle bound to an isolated polypeptide capable of binding to a viral capsid of the invention exhibits at least 60% decrease in transduction efficiency compared to an appropriate control wildtype virus particle. In some embodiments, a virus particle bound to an isolated polypeptide capable of binding to a viral capsid of the invention exhibits at least 70% decrease in transduction efficiency compared to an appropriate control wildtype virus particle. In some embodiments, a virus particle bound to an isolated polypeptide capable of binding to a viral capsid of the invention exhibits at least 80% decrease in transduction efficiency compared to an appropriate control wildtype virus particle. In some embodiments, a virus particle bound to an isolated polypeptide capable of binding to a viral capsid of the invention exhibits at least 90% decrease in transduction efficiency compared to an appropriate control wildtype virus particle. In some embodiments, a virus particle bound to an isolated polypeptide capable of binding to a viral capsid of the invention exhibits at least 95% decrease in transduction efficiency compared to an appropriate control wildtype virus particle. In some embodiments, a virus particle bound to an isolated polypeptide capable of binding to a viral capsid of the invention exhibits at least 99% decrease in transduction efficiency compared to an appropriate control wildtype virus particle. In some embodiments, transduction of a control cell by a virus particle comprising a recombinant virus particle molecule as described herein is abolished, e.g., undetectable, e.g., via methods measuring expression of the nucleotide of interest, e.g., reporter assays, etc.

Conversely, a virus particle bound to a virus adaptor molecule comprising a ligand may be able to infect a target cell, e.g., has a partially or completely restored capacity to target and bind a reference cell naturally permissive to transduction compared to that of a reference viral capsid, e.g., a capsid comprising a reference viral capsid protein, e.g., a wildtype control viral capsid protein. In some embodiments, a virus particle bound to a virus adaptor molecule comprising an isolated polypeptide capable of binding to a viral capsid and a ligand of the invention exhibits a transduction efficiency that is at least 10% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 20% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 30% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 40% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 50% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 60% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 70% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 80% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 90% higher than the transduction efficiency of an appropriate control wildtype viral capsid. In some embodiments, a virus particle bound to a virus adaptor molecule comprising an isolated polypeptide capable of binding to a viral capsid and a ligand of the invention exhibits a transduction efficiency that is at least 100% higher than the transduction efficiency of an appropriate control wildtype viral capsid.

In some embodiments, a virus particle covalently bound to an isolated polypeptide comprising one or more heterologous cysteine residues capable of covalently binding to a viral capsid of the invention exhibits at least 10% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 20% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 30% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 40% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 50% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 60% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 70% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 80% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 90% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 95% decrease in transduction efficiency compared to an appropriate control wildtype virus particle. In some embodiments, a virus particle covalently bound to an isolated polypeptide comprising one or more heterologous cysteine residues capable of covalently binding to a viral capsid of the invention exhibits at least 99% decrease in transduction efficiency compared to an appropriate control wildtype virus particle. In some embodiments, transduction of a control cell by a virus particle comprising a recombinant virus particle molecule as described herein is abolished, e.g., undetectable, e.g., via methods measuring expression of the nucleotide of interest, e.g., reporter assays, etc.

Conversely, a virus particle comprising a covalently bound virus adaptor molecule comprising a ligand is able to infect a target cell, e.g., has a partially or completely restored capacity to target and bind a reference cell naturally permissive to transduction compared to that of a reference viral capsid, e.g., a capsid comprising a reference viral capsid protein, e.g., a wildtype control viral capsid protein. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 10% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 20% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 30% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 40% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 50% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 60% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 70% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 80% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 90% higher than the transduction efficiency of an appropriate control wildtype viral capsid. In some embodiments, a virus particle covalently bound to a virus adaptor molecule comprising an isolated polypeptide comprising one or more heterologous cysteine residues capable of covalently binding to a viral capsid and a ligand of the invention exhibits a transduction efficiency that is at least 100% higher than the transduction efficiency of an appropriate control wildtype viral capsid.

### Identification of Residues Suitable for Mutation in Capsid Proteins and Binder Proteins

In order to identify pairs of residues which could be mutated into cysteines capable of forming disulfide bonds, the structures of viruses bound to a receptor or ligand were retrieved from public databases such as PDB (e.g. AAV1 structure in complex with the PKD2 domain of AAVR, PBD entry 6jcq). Residues lining the interface between the two partners were examined to identify pairs of (i) a residue on the viral particle and (ii) a residue on the receptor or ligand, such that:
when mutated in silico to cysteine residues using software such as Pymol or Chimera, the side chains would not induce a major steric clash which would disrupt the interface (i.e. the side chain of the cysteine residues would not occupy a region of space where another residue were already present);
the distance of the beta carbons of the cysteines replacing residues (i) and (ii) would be less than 5.5 Å;
an orientation would exist of the side chains of the cysteines residues (i) and (ii), obtained by rotation of the side chain around C(α)-C(β), the such that the distance of the sulfur atoms at the gamma positions of the side chains would be less than 2.5 Å; the angles formed by the atoms the C(β)1-S(γ)1-S(γ)2, S(γ)1-S(γ)2-C(β) would not be markedly different (>40%) from the optimal angles observed in naturally occurring disulfide bonds, as indicated in the literature (e.g. Dombkowski et al., Protein disulphide engineering, FEBS Letters Volume 588, Issue 2, 21 Jan. 2014, Pages 206-212).

To take into account that the flexibility of proteins is not correctly represented by static structures, a discretionary deviation from the reference parameters was allowed for surface residues displaying minimal structural constraints.

An adaptor protein (A) can be derived from a viral binding protein (B) capable of interacting with a viral particle (V) such that:
1) The binding protein (B) can bind to a virus (V) with affinity Kd<100 μM, as measured by standard methods in the art
2) At least one pair of residues X and Y exist, where X is part of the binding protein B and Y is part of the viral particle V, such that their respective β carbons are less than 5.5 Å apart when the binding protein B forms a complex with the viral particle V;
3) Residues X and Y can be mutated to cysteines such that the cysteine side chains do not induce a major steric clash which would disrupt the interface (i.e. the side chain of the cysteine residues would not occupy a region of space where another residue is already present);
4) An orientation exists for the side chains of residues X and Y, after mutation to cysteines, obtained by rotation of the side chain around C(α)-C(β), such that the distance of the sulfur atoms at the gamma positions of the side chains are less than 2.5 Å apart; and/or
5) The angles formed by the atoms the C(β)1-S(γ)1-S(γ)2, S(γ)1-S(γ)2-C(β) would not be markedly different (for example are not more than 40% greater) than the optimal angles observed in naturally occurring disulfide bonds, as indicated in the literature (e.g. Dombkowski et al., Protein disulphide engineering, FEBS Letters Volume 588, Issue 2, 21 Jan. 2014, Pages 206-212).
   When one or more of these criteria are fulfilled, the adapter protein (A) obtained from the binding protein (B) by mutation of X to a cysteine, and the modified virus (V′) obtained from V by mutation of Y to a cysteine, are a candidate adapter protein-virus pair that can be expected with reasonable likelihood to bind to each other covalently, in accordance with the present invention.

In some embodiments of the present invention, candidate adapter protein-virus pairs can be selected based on one or more of the criteria above. In some embodiments of the present invention, the candidate adapter protein-virus pair meets at least 1 requirement in the list above. In some embodiments of the present invention, the candidate adapter protein-virus pair meets at least 2 requirements in the list above. In some embodiments of the present invention, the candidate adapter protein-virus pair meets at least 3 requirements in the list above. In some embodiments of the present invention, the candidate adapter protein-virus pair meets at least 4 requirements in the list above. In some embodiments of the present invention, the candidate adapter protein-virus pair meets all 5 requirements in the list above.

In some embodiments of the present invention, residues in a viral capsid and/or in an isolated polypeptide suitable for binding to a viral capsid can be selected for mutation to cysteine residues by using a method as set herein. Means for mutating amino acid residues to a cysteine residue are known to the skilled person, for example via genetic or chemical means.

The present invention is further illustrated in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLES

### Example 1—Formation of a Disulfide Bond Between Residue Cys590 of AAV1(Asp590Cys) Mutant and Cys425 of sfGFP-PKD2(Ser425Cys) Adaptor Fusion Protein

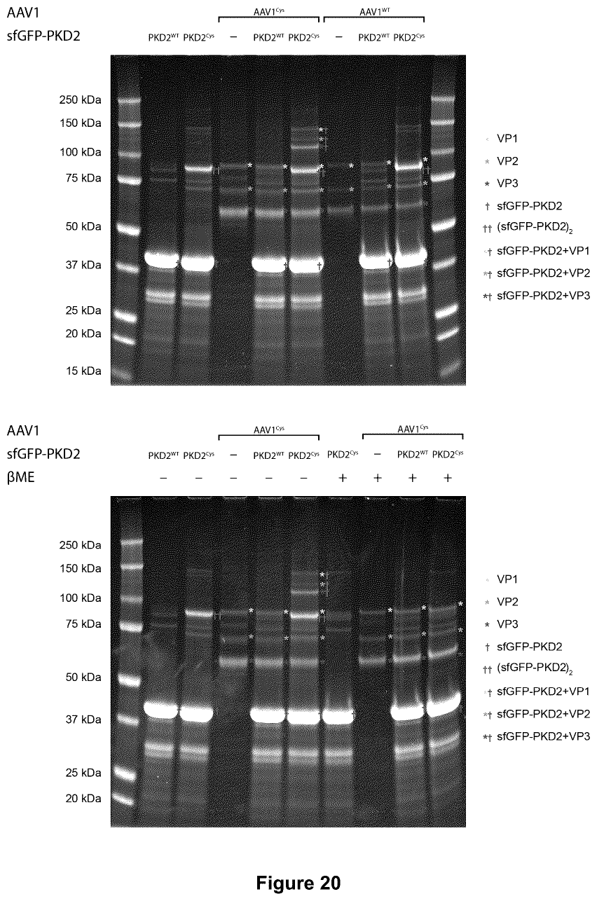

See FIG. 20. Top gel—2 μL of viral, either wildtype (AAV1WT in the legend) or Asp590Cys (AAV1Cys in the legend) were mixed with 1.5 μL of a 100 μM stock of sfGFP-PKD2 fusion protein, either wt (PKD2WT in the legend) or Ser425Cys (PKD2Cys in the legend), in a final reaction volume of 10 μL (20 mM Tris HCl pH 8, 100 mM NaCl), in the combinations indicated in the figure. The reactions were incubated at room temp for 1 h, then were run on a 4-12% NuPAGE for 1 h at 200V. The appropriate individual components were run as a control as well. The gel loading dye did not contain reducing agents.

See FIG. 20. Bottom gel—4 μL of viral stock, either wildtype (AAV1WT in the legend) or Asp590Cys (AAV1Cys in the legend) were mixed with 3 μL of a 100 μM stock of sfGFP-PKD2 fusion protein, either wt (PKD2WT in the legend) or Ser425Cys (PKD2Cys in the legend), in a final reaction volume of 20 μL (20 mM Tris HCl pH 8, 100 mM NaCl), in the combinations indicated in the figure. The reaction was incubated at room temp for 1 h, then the samples were split in two. Half of the reaction was mixed with a non-reducing loading dye (left lanes), and half of the reaction was mixed with a reducing loading dye containing @ME. The samples were run on a 4-12% NuPAGE for 1 h at 200V. The appropriate individual components were run as a control as well.

The gel shows the formation of bands corresponding to sfGFP-PKD2+AAV1, sfGFP-PKD2+VP2 and sfGFP-PKD2+VP3, migrating between 150 kDa and 100 kDa, which can only be observed when both the AAV1 capsid and the sfGFP-PKD2 adaptor protein contain a cysteine residue, and which are labile in the presence of reducing agents, consistent with disulfide-bonded heterodimers (FIG. 20).

### Example 2—Purification of Covalently Bound AAV1 CAR-V Complexes

AAV1(Asp590Cys) virus was incubated overnight in PBS in the presence of 50 μM sfGFP-PKD2(Ser425Cys) fusion protein—where PKD2(Ser425Cys) refers to the PKD2 domain of the AAVR(Ser425Cys)) mutant (fusion protein 50 μM).

After incubation, the reaction was subject to ultracentrifugation using an iodixanol step gradient, in order to separate viral particles from the unreacted excess sfGFP-PKD2(Ser425Cys) fusion polypeptide. After ultracentrifugation, particles that partitioned in the 40% iodixanol fraction were collected, buffer exchanged and run on a 4-12% SDS PAGE.

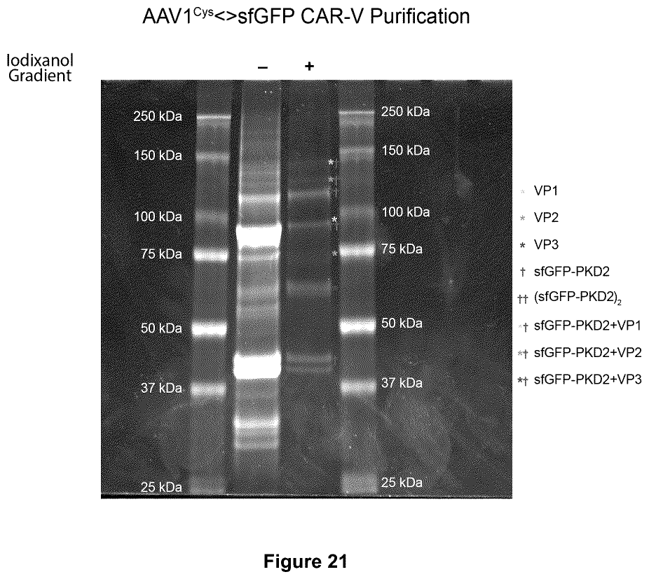

The gel shows that the gradient was effective at purifying out the majority of the unbound sfGFP-PKD2(Ser425Cys) adaptor protein (compare bands between 37 kDa and 50 kDa, and between 75 kDa and 100 kDa). Moreover, the gel shows that stably-bound heterodimers of the PKD2 mutant fusion protein and the viral capsid proteins VP1, VP2 and VP3 could be recovered (bands above 100 kDa). This proved the successful formation of a covalently bound AAV1Cys< >sfGFP particle (FIG. 21).

### Example 3—Tropism of AAV1 Covalently Bound to an Adaptor

HEK293T cells were seeded in a 24 well plate. When the cells reached approx. 60% confluency, two wells were transfected with a plasmid expressing a chimeric sfGFP receptor, in the form of a membrane-bound anti-sfGFP nanobody (right column), while the other wells were not transfected (left column). Cells were incubated for 24 h at 37° C. to enable the expression of the chimeric receptor in the transfected cells. Subsequently, cells were either infected with AAV1(Asp590Cys)< >sfGFP (CAR-V), or with AAV1(Asp590Cys). Both viruses carried the gene encoding the red-fluorescent protein mScarlet. Images show phase contrast (gray) or mScarlet (red).

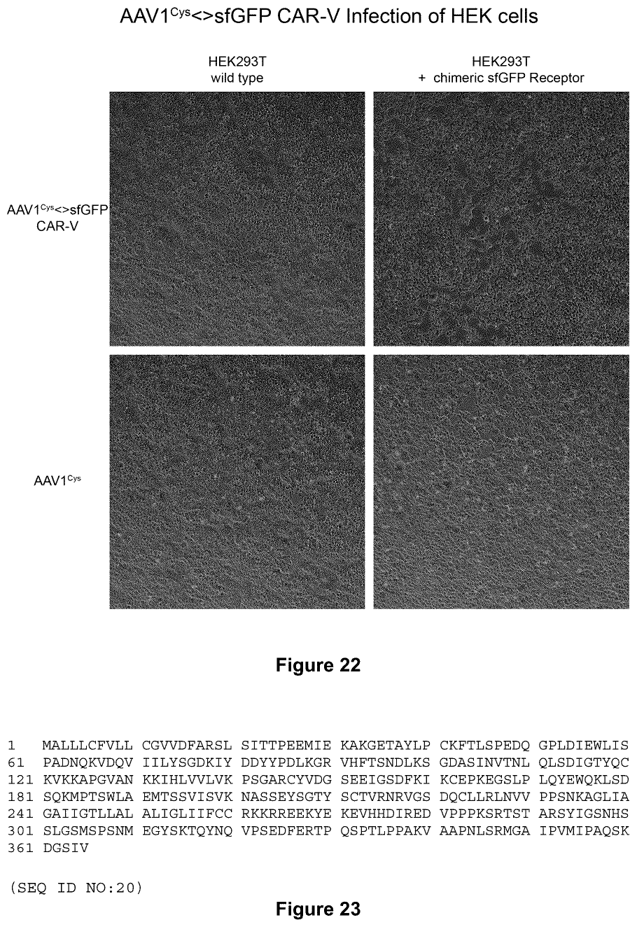

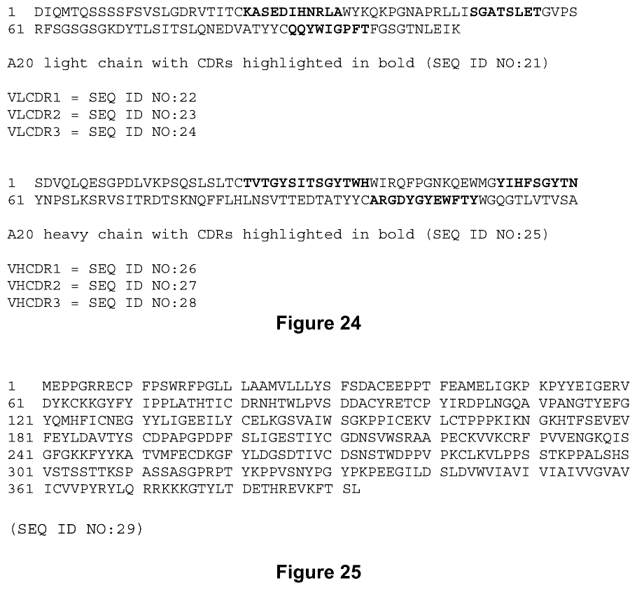

The data shows that AAV1(Asp590Cys)< >sfGFP CAR-V (as described in Example 1) has reduced tropism towards HEK293T cells that are not expressing the chimeric sfGFP receptor; but that infectivity is recovered when the target cells express the chimeric receptor. The data further show that HEK293T cells are highly susceptible to infection with AAV1(Asp590Cys) without the adaptor protein. These data demonstrate that the adaptor protein reduces the infectivity of AAV1 towards cells that do not express the corresponding chimeric receptor, but restores infectivity towards cells that do express the corresponding chimeric receptor (FIG. 22).

### Example 4—Use of Small Molecule Ligands

It is possible to generate adaptor proteins by taking an isolated polypeptide comprising one or more cysteine residues capable of covalently binding to a viral capsid and reacting it with a targeting molecule with high specificity for a target cell. The reaction may be performed using NHS or maleimide chemistry, or it may be performed by incorporating a non-canonical amino acid with a reactive group (such as an azide or an alkyne or tetrazine or trans-cyclooctene) at a specific location in the viral capsid-binding molecule.

It may also be performed by fusing the isolated polypeptide comprising one or more cysteine residues capable of covalently binding to a viral capsid with a specific peptide sequence that is capable of binding to the targeting molecule, such as a split intein, SpyTag, the tetracysteine FCM motif (FLNCCPGCCMEP) or the ybbR motif (TVLDSLEFIASKLA). In these cases, the targeting molecule would be designed to contain the other side of the reactive element, such as the other side of the split intein; SpyCatcher; a fluorescein arsenical hairpin, or coenzyme A, respectively.

The targeting molecule may then be chosen to bind to a specific receptor. In some instances, the targeting molecule may be a protein, such as an antibody against a receptor expressed on the target cell type. In the case of antibodies, the antibody may be modified with an alkyne group and then reacted via click chemistry to an azide group on a viral capsid molecule.

In some instances, the targeting molecule may be a small molecule. For example, by attaching a neurotransmitter such as serotonin or dopamine to the viral capsid, it would be possible to create an adaptor molecule that, when combined with a virus particle, would generate a virus particle specific to cells that express the serotonin or dopamine receptors, respectively. This approach (i.e., creating an adaptor protein comprising an isolated polypeptide comprising one or more cysteine residues capable of covalently binding to a viral capsid and a reactive group and then reacting it to a targeting molecule) would be particularly useful if the targeting molecule cannot be expressed as a fusion with the isolated polypeptide comprising one or more cysteine residues capable of covalently binding to a viral capsid (e.g. in the case where the targeting molecule is not a single chain protein, or is a small molecule), or in the case where the fusion of the targeting molecule to the isolated polypeptide comprising one or more cysteine residues capable of covalently binding to a viral capsid cannot be purified efficiently.

### Example 5—Use of Different Viruses

### Adenoviruses

The invention can be implemented using adenoviruses for delivery of payloads that are too large for AAVs. For example, adenoviruses (AdVs) have been used in the AstraZeneca COVID-19 vaccine to deliver the full-length spike protein, and adenoviruses have been in trials for several other gene therapies. As in the case of AAVs, cell-type-specific AdVs would enable more specific, more efficient, and less-toxic gene therapies. Some adenoviruses are specific for the coxsackievirus and adenovirus receptor (CXADR). Thus, an adaptor molecule comprising a fusion of CXADR to a ligand could be used for retargeting adenoviruses to specific cell types. In some embodiments of the invention, an adaptor molecule comprises of a fusion of at least a portion of CXADR and a ligand. Some adenoviruses are specific for CD46. Thus, an adaptor molecule comprising a fusion of a portion or the full-length of CD46, to a ligand could be used for retargeting adenoviruses to specific cell types. In some embodiments of the invention, an adaptor molecule comprises of a fusion of at least a portion of CD46 and a ligand. The invention could be implemented using an adenovirus-CXADR CAR-V or an adenovirus-CD46 CAR-V or an AAV-A20 antibody CAR-V. In some embodiments of the invention the CXADR protein may comprise a portion of SEQ ID NO:20. In some embodiments of the invention the CXADR protein may comprise the full-length sequence of SEQ ID NO:20.

### Lentiviruses

The invention can be implemented using lentiviruses to deliver payloads that are intended for genomic integration. For example, lentiviruses are often used for cell engineering in vitro, because their payloads integrate into the target cells, thus allowing for the creation of stable cell lines with specific genetic insertions. Cell-type-specific lentiviral vectors could be used to increase the specificity and efficiency of gene editing steps in cell therapies. Lentiviruses are highly specific for the CD4 receptor; thus an adaptor molecule consisting of a portion of the CD4 receptor fused to a ligand could facilitate retargeting of lentiviral vectors to specific cell types.

### Combinations of Residues on Viruses and Binders

For the combination of human Adenovirus D37 fiber protein and human (h) CxAdR (crystal structure accession number in PDB 2j12, see Seiradake et al., Structural and Mutational Analysis of Human (h) Ad37 and Canine Adenovirus 2 Fiber Heads in Complex with the D1 Domain of Coxsackie and Adenovirus Receptor, JBC, Volume 281, Issue 44, 3 Nov. 2006, Pages 33704-33716), covalently bound adenovirus (hAdV) CAR-V can be obtained by using a hAdV-D37 containing the mutations below, in pair with an adaptor fusion protein containing at least a portion of hCxAdR, where the hCxAdR contains the corresponding point mutations below [numbering as appearing in the PDB file]:
Val226Cys on hAdV-D37 and Val70Cys in hCxAdR
Ser193Cys on hAdV-D37 and Glu56Cys in hCxAdR
Ser274Cys on hAdV-D37 and Val128Cys in hCxAdR

In some embodiments of the invention, the isolated polypeptides or adaptor proteins of the invention may comprise at least a portion of hCxAdR in combination with hAdV-D37 comprising one or more of the point mutations set out above. In some embodiments of the invention, the isolated polypeptides or adaptor proteins of the invention may comprise at least a portion of hCxAdR in combination with human adenovirus 5 (e.g. comprising hAdV-D37) wherein the hCxAdR comprises a Val70Cys mutation and AdV-5 comprises a Val441Cys mutation.

Other adenoviruses may not bind to CxAdR, but to other cellular receptors, including CD46 (see Seiradake et al., Structural and Mutational Analysis of Human Ad37 and Canine Adenovirus 2 Fiber Heads in Complex with the D1 Domain of Coxsackie and Adenovirus Receptor, JBC, Volume 281, Issue 44, 3 Nov. 2006, Pages 33704-33716). Accordingly, the invention may be implemented using combinations of an adenovirus capsid or viral particle and another suitable adenovirus binding protein, such as CD46.

The structure of the fiber protein of hAdV11p in complex with hCD46 (PDB entry 2039, see Persson et al., Adenovirus type 11 binding alters the conformation of its receptor CD46, Nature Structural & Molecular Biology volume 14, pages 164-166 (2007)) indicates that that covalently bound hAdV CAR-V can be obtained by using a hAdV-11p containing the point mutation below, in pair with an adaptor fusion protein containing a fragment of hCD46, where the hCD46 contains the corresponding point mutation below:
Asn283Cys on hAdV-11p and Thr42Cys in hCD46

In some embodiments of the invention, the isolated polypeptides or adaptor proteins of the invention may comprise at least a portion of CD46 in combination with hAdV-11p comprising the point mutation set out above.

AAV2 is capable of binding to the A20 neutralizing antibody (PDB entry 3j1s (see McCraw et al., Structure of adeno-associated virus-2 in complex with neutralizing monoclonal antibody, Volume 431, Issues 1-2, 15-30 Sep. 2012, Pages 40-49).

Covalently bound AAV2 CAR-V with A20 antibody can be obtained by using an AAV2 containing the mutation below, in pair with an adaptor fusion protein containing a portion of the A20 neutralising antibody (nAb), where the A20 nAb contains the corresponding mutation below:
AAV2(Ser264Cys) to A20 Antibody(Heavy chain Tyr102Cys→This is in CDR3 of A20)
AAV2(Val708Cys) to A20 Antibody(Heavy chain Ser56Cys→This is in CDR2 of A20)
AAV2(Asn717Cys) to A20 Antibody(Light chain Ile93Cys→This is in CDR3 of A20)

In some embodiments of the invention, the isolated polypeptides or adaptor proteins of the invention may comprise at least a portion of the A20 antibody in combination with AAV2 comprising one or more of the point mutations set out above. In some embodiments of the invention the AAV2 comprises a sequence having at least 85%, 90%, 95%, 99% or 100% identity to SEQ ID NO:8 and the portion of the A20 antibody comprises one or more sequences having at least 85%, 90%, 95%, 99% or 100% identity to any one of SEQ ID NOs: 21-28.

AAV5 is capable of binding the PKD1 domain of AAVR. Covalent binding between AAV5 and the PKD1 domain of AAVR can be obtained by using an AAV5 containing one or more of the mutations below, in pair with an adaptor fusion protein containing at least a portion of the PKD1 domain of AAVR, wherein the PKD1 domain of AAVR contains the corresponding mutation below:
Mutation Gln697Cys on AAV5 and Ser356Cys on PKD1;
Mutation Leu543Cys on AAV5 and Leu376Cys on PKD1

In some embodiments of the invention, the isolated polypeptides or adaptor proteins of the invention may comprise at least a portion of the PKD1 domain of AAVR in combination with AAV5 comprising one or more of the point mutations set out above. In some embodiments of the invention the AAV5 comprises a sequence having at least 85%, 90%, 95%, 99% or 100% identity to SEQ ID NO: 11 and the PKD1 domain of AAVR comprises a sequence having at least 85%, 90%, 95%, 99% or 100% identity to SEQ ID NO:2.

Human Adenovirus B3 (through its fibre protein, Uniprot sequence P04501) is capable of binding desmoglein 2 (DSG2, Uniprot sequence ID: Q14126). Covalent binding between Human Adenovirus B3 and desmoglein 2 can be obtained by using a Human Adenovirus B3 containing one or more of the mutations below, in pair with an adaptor fusion protein containing at least a portion of desmoglein 2, wherein desmoglein 2 contains the corresponding mutation below:
Mutation Tyr147Cys on the fibre protein and mutation His175Cys on DSG2;
Mutation Asn188Cys on the fibre protein and mutation Thr226Cys on DSG2;
Mutation Asn192Cys on the fibre protein and mutation Ala174Cys on DSG2;
Mutation Asp261Cys on the fibre protein and mutation Lys362Cys on DSG2;
Mutation Phe265Cys on the fibre protein and mutation Ser366Cys on DSG2

In some embodiments of the invention, the isolated polypeptides or adaptor proteins of the invention may comprise at least a portion of desmoglein 2 in combination with Human Adenovirus B3 comprising one or more of the point mutations set out above. In some embodiments of the invention the Human Adenovirus B3 comprises a sequence having at least 85%, 90%, 95%, 99% or 100% identity to SEQ ID NO:30 and the desmoglein 2 comprises a sequence having at least 85%, 90%, 95%, 99% or 100% identity to SEQ ID NO:31. In some embodiments the isolated polypeptides or adaptor proteins of the invention may comprise at least a portion of desmoglein 2 in combination with Human Adenovirus B3 wherein the Human Adenovirus B3 comprises a Asn192Cys mutation on the fibre protein and the portion of desmoglein 2 comprises a Ala174Cys mutation.

### Example 6—sfGFP-PKD2 Fusion Protein

In a 24 well plate HEK293FT cells were seeded in wells A, B and C and waited for them to reach 70-80% confluency. The cells were then transfected in all 3 wells with a plasmid enabling the expression of a membrane-anchored anti-sfGFP nanobody. 24h after transfection, 5 μL of sfGFP-PKD2 fusion protein (about 2 mg/mL) was added to well A3, incubated 30 min to enable capture of the fusion protein by the nanobody, then the unbound fusion protein washed off and the cell medium changed.

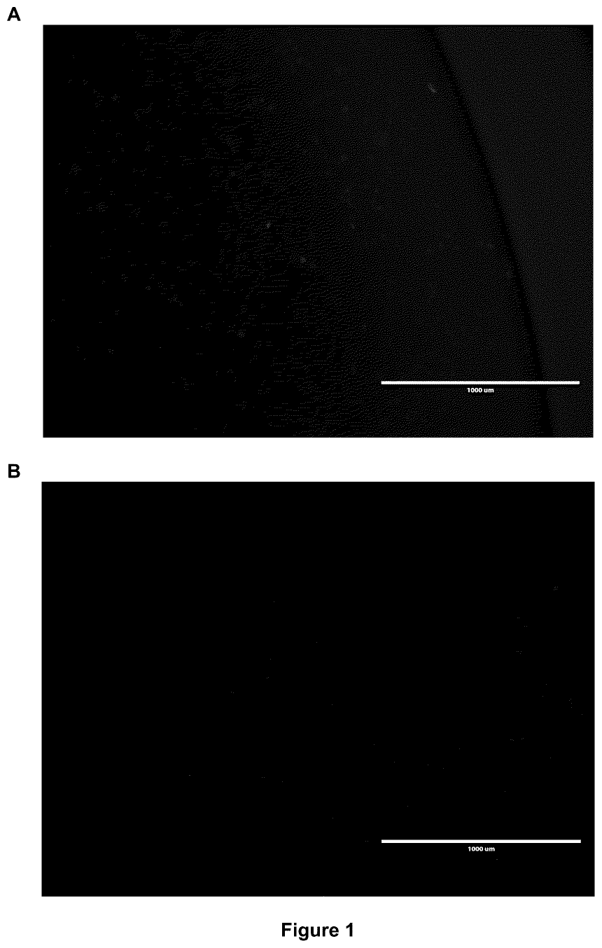

Well A (FIG. 1A) was infected with AAV2 wildtype carrying the mScarlet gene. Wells B and C (FIGS. 1B and C) were infected with AAV2 virus Arg585Ala Arg588Ala carrying the mScarlet gene. 24h after infection, the cells were imaged to visualise mScarlet. The images shows that the sfGFP-PKD2 fusion protein enhances the infectivity of AAV2 Arg585Ala Arg588Ala in cells expressing the synthetic membrane-anchored anti-sfGFP nanobody (FIG. 1).

### Example 7—SKBR3 Cells with DARPin-PKD2 Fusion Proteins

Two 35 mm dishes were seeded with SKBR3 cells. When the cells reached about 60% confluency, the medium was replaced with 1 mL of DMEM+10% FBS, and 100 μL of DARPin-PKD2 (2 μM)—where the DARPin ligand was a high affinity binder for Her2—was added to one of the dishes and the other kept as a control. The wells were incubated for 30 min to enable the DARPin domain of the DARPin-PKD2 fusion protein to bind to the Her2 receptor which is expressed endogenously by the SKBR3 cell line, then the medium was removed, the cells washed with PBS and fresh medium added.

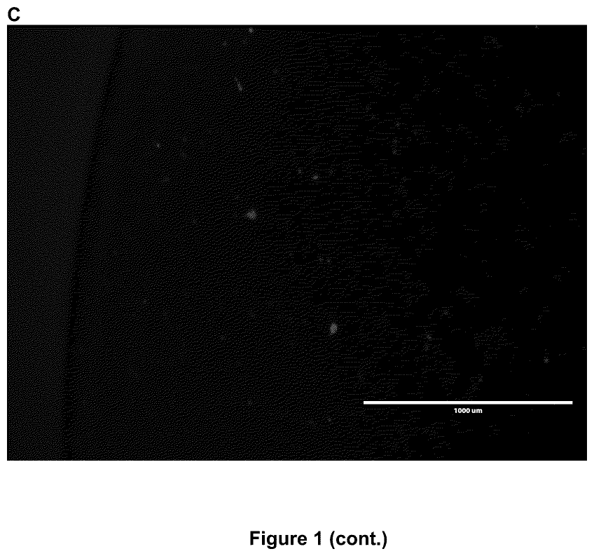

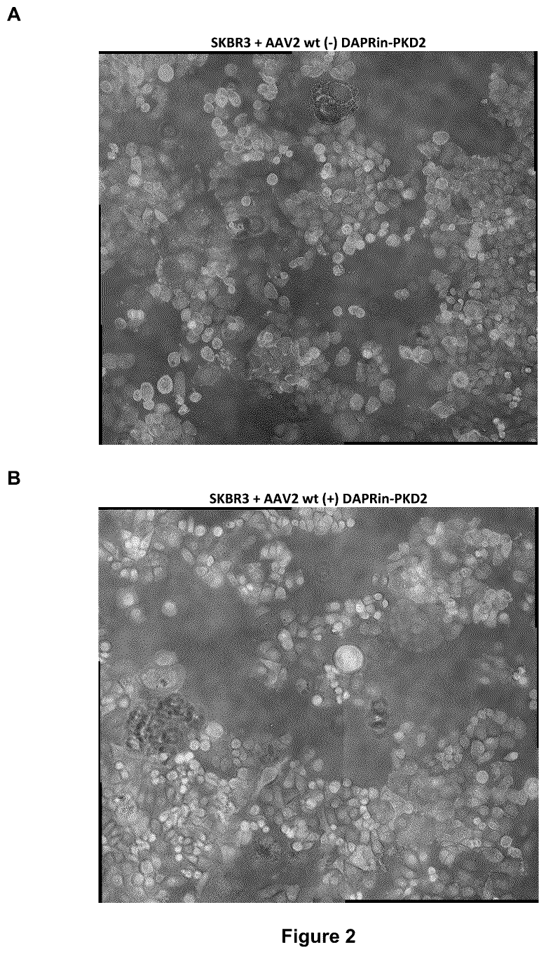

Both dishes were then infected with AAV2 wildtype carrying the mScarlet gene. After 24 h, the cells were fixed for immunostaining (DNA=blue, Her2=green, mScarlet=red, Phase Contrast=grey). The images show that the DARPin-PKD2 fusion protein greatly enhances infectivity of AAV2 wildtype in SKBR3 cells (FIG. 2).

### Example 8—mScarlet-PKD2 Fusion Protein Blocks Infectivity of AAV2

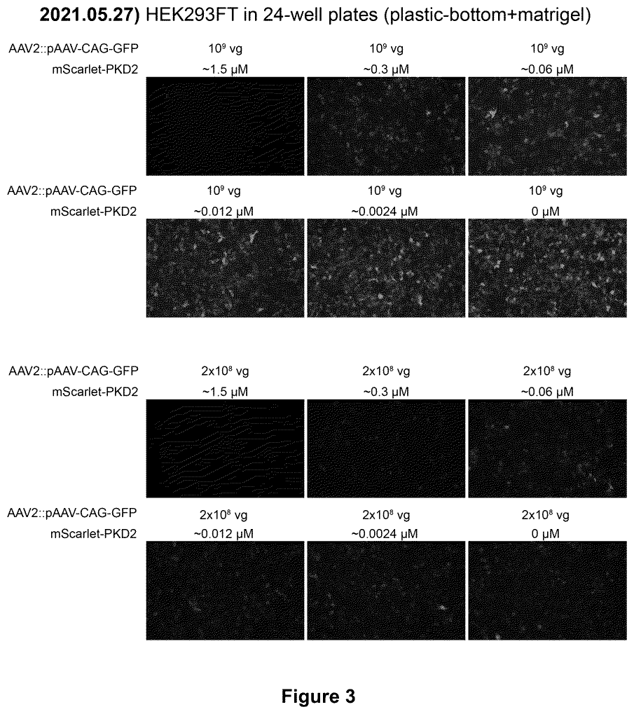

HEK293FT cells were seeded in a 24 well plate coated with Matrigel and cultured until the cells reached about 90% confluency. mScarlet-PKD2 fusion protein (at the concentration indicated in FIG. 3) was mixed with AAV2 wildtype carrying the GFP gene under a strong promoter (at the concentration indicated in FIG. 3), varying the concentration of the fusion protein in each sample while keeping the amount of virus fixed. After 10 min incubation, the cells were infected with the mScarlet-PKD2+AAV2 mix. After 24 h, the cells were imaged for GFP expression as a marker for infection.

The data show that the presence in solution of mScarlet-PKD2 fusion protein inhibits the infectivity of AAV2 wildtype in a concentration-dependant manner, suggesting that the PKD2 domain of the fusion protein can effectively interact with the virus and outcompete the cellular AAVR for binding, preventing the infection (FIG. 3).

### Example 9—Binding of the PKD2 Fusion Proteins to AAV2

10 μL of commercially sourced AAV2 virus (10\hat{ }13 vg/mL) was immobilised onto a CM3 chip to perform surface plasmon resonance (SPR) by amine coupling. The SPR signal of the AAV2 channel was measured relative to a reference channel given by increasing concentrations of sfGFP-PKD2 fusion protein.

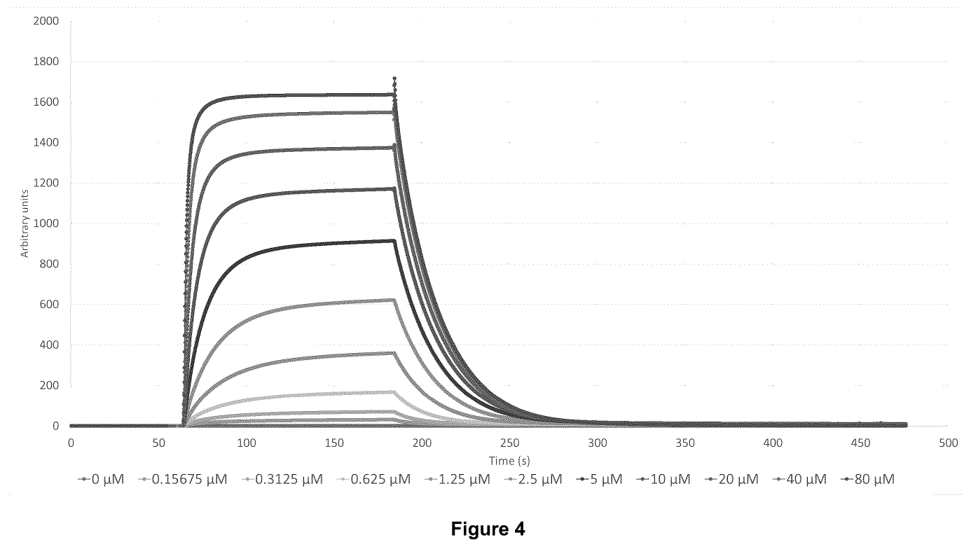

The SPR signal confirms the interaction between the immobilized AAV2 and the fusion protein with measured affinity of around 6 μM (FIG. 4).

### Example 10—Binding of the PKD2 Fusion Proteins to AAV1

10 μL of commercially sourced AAV1 virus (10\hat{ }13 vg/mL) was immobilised onto a CM3 chip to perform surface plasmon resonance (SPR) by amine coupling. The SPR signal of the AAV2 channel was measured relative to a reference channel given by increasing concentrations of sfGFP-PKD2 fusion protein.

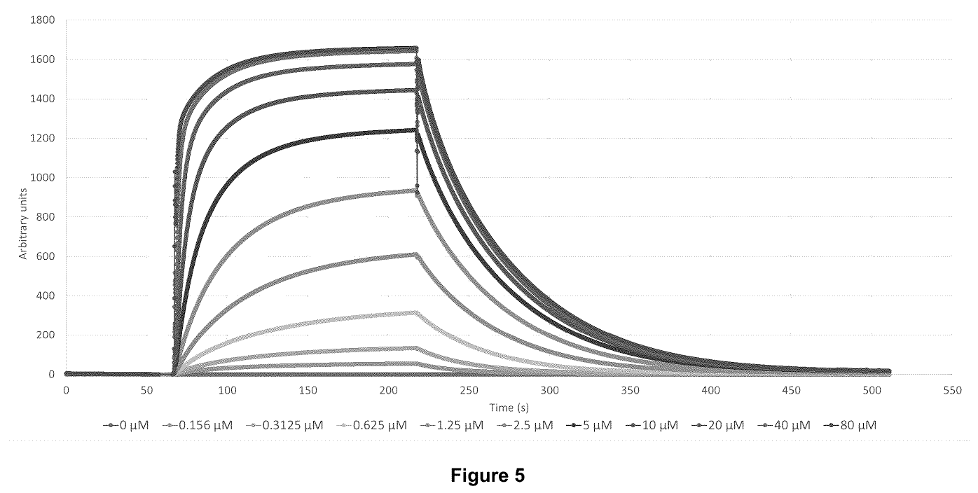

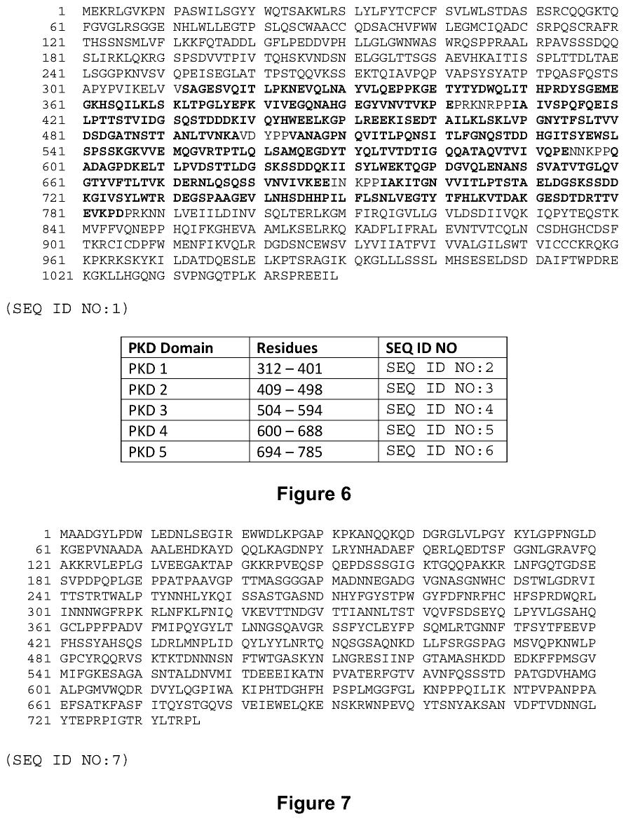

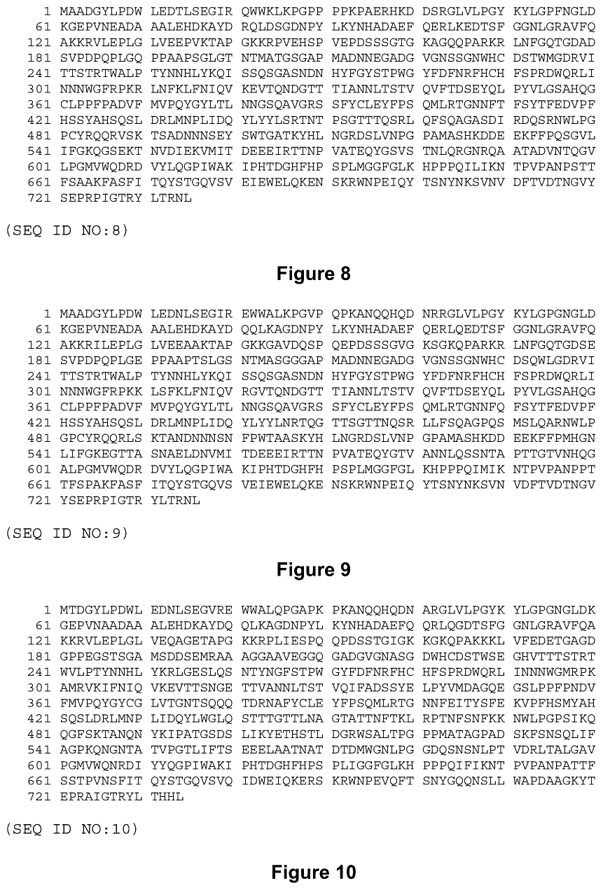

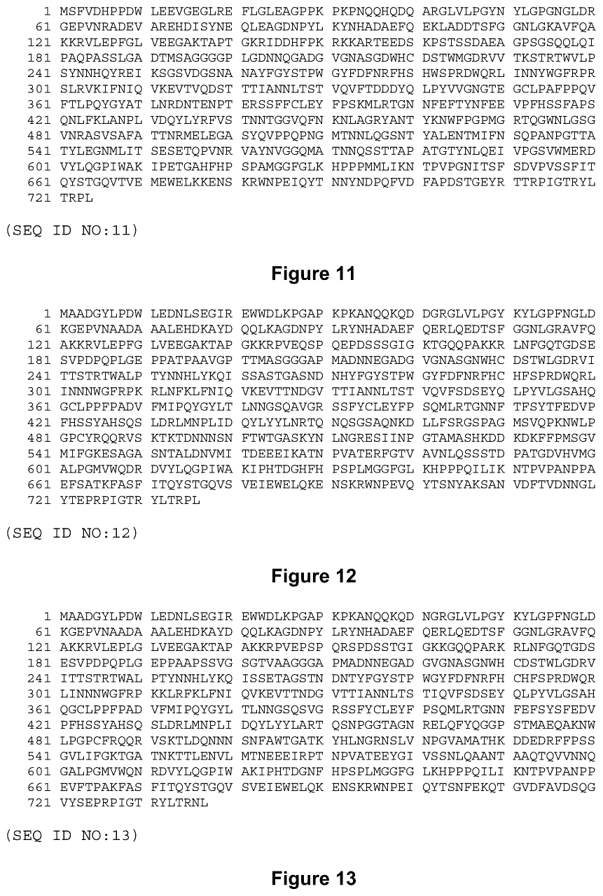

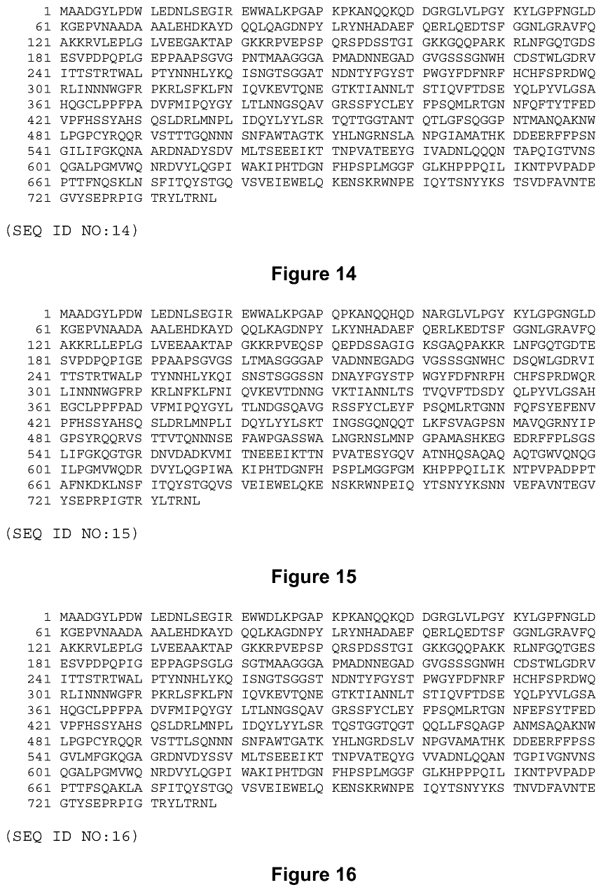

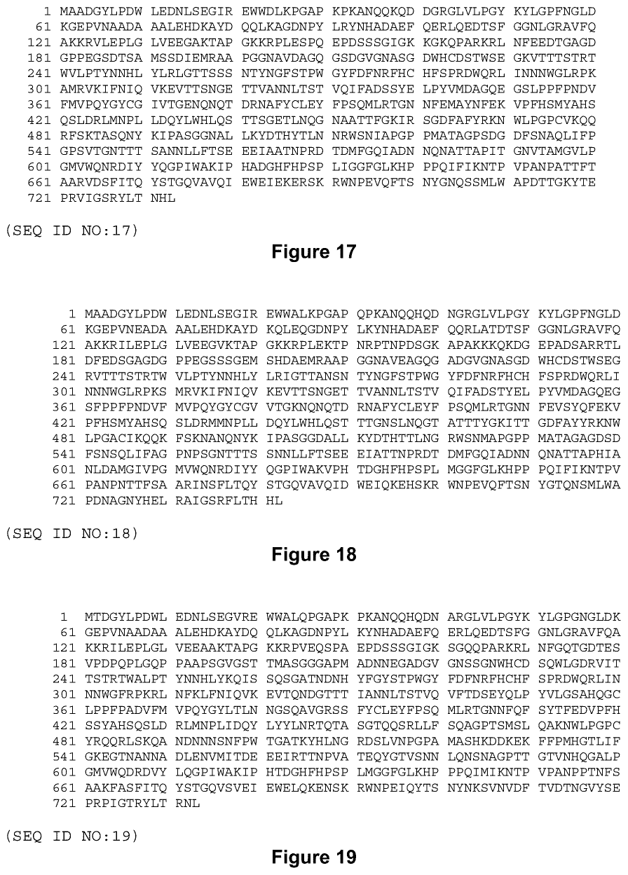

The SPR signal confirms the interaction between the immobilized AAV1 and the fusion protein with measured affinity of around 2 μM (FIG. 5).

### Example 11—Use of Small Molecule Ligands

It is possible to generate adaptor proteins by taking an isolated polypeptide capable of binding to an adeno-associated virus (AAV) capsid (e.g. a PKD2 domain-containing protein) and reacting it with a targeting molecule with high specificity for a target cell. The reaction may be performed using NHS or maleimide chemistry, or it may be performed by incorporating a non-canonical amino acid with a reactive group (such as an azide or an alkyne or a tetrazine or a trans-cyclooctene) at a specific location in the isolated polypeptide capable of binding to an adeno-associated virus (AAV) capsid.

It may also be performed by fusing the isolated polypeptide capable of binding to an adeno-associated virus (AAV) capsid molecule with a specific peptide sequence that is capable of binding to the targeting molecule, such as a split intein, SpyTag, the tetracysteine FCM motif (FLNCCPGCCMEP) or the ybbR motif (TVLDSLEFIASKLA) (see Fernandes, D. D., et al. Characterization of Fluorescein Arsenical Hairpin (FlAsH) as a Probe for Single-Molecule Fluorescence Spectroscopy. Sci Rep 7, 13063 (2017). In these cases, the targeting molecule would be designed to contain the other side of the reactive element, such as the other side of the split intein; SpyCatcher; a fluorescein arsenical hairpin, or coenzyme A, respectively.

The targeting molecule may then be chosen to bind to a specific receptor. In some instances, the targeting molecule may be a protein, such as an antibody against a receptor expressed on the target cell type. In the case of antibodies, the antibody may be modified with an alkyne group and then reacted via click chemistry to an azide group on an isolated polypeptide capable of binding to an adeno-associated virus (AAV) capsid.

In some instances, the targeting molecule may be a small molecule. In some instances, the isolated polypeptide capable of binding to an adeno-associated virus (AAV) capsid is a PKD2 domain-containing protein. For example, by attaching a neurotransmitter such as serotonin or dopamine to an isolated polypeptide capable of binding to an adeno-associated virus (AAV) capsid, it would be possible to create an adaptor molecule that, when combined with an AAV, would generate an AAV specific to cells that express the serotonin or dopamine receptors, respectively. This approach (i.e., creating an adaptor molecule comprising an isolated polypeptide capable of binding to an adeno-associated virus (AAV) capsid and a reactive group and then reacting it to a targeting molecule) would be particularly useful if the targeting molecule cannot be expressed as a fusion with an isolated polypeptide capable of binding to an adeno-associated virus (AAV) capsid (e.g. in the case where the targeting molecule is not a single chain protein, or is a small molecule), or in the case where the fusion of the targeting molecule to an isolated polypeptide capable of binding to an adeno-associated virus (AAV) capsid cannot be purified efficiently.

### Example 12—Further Exemplifications of the Present Invention

The inventors have identified a number of aspects of the present invention which give rise to particularly beneficial features of the invention:

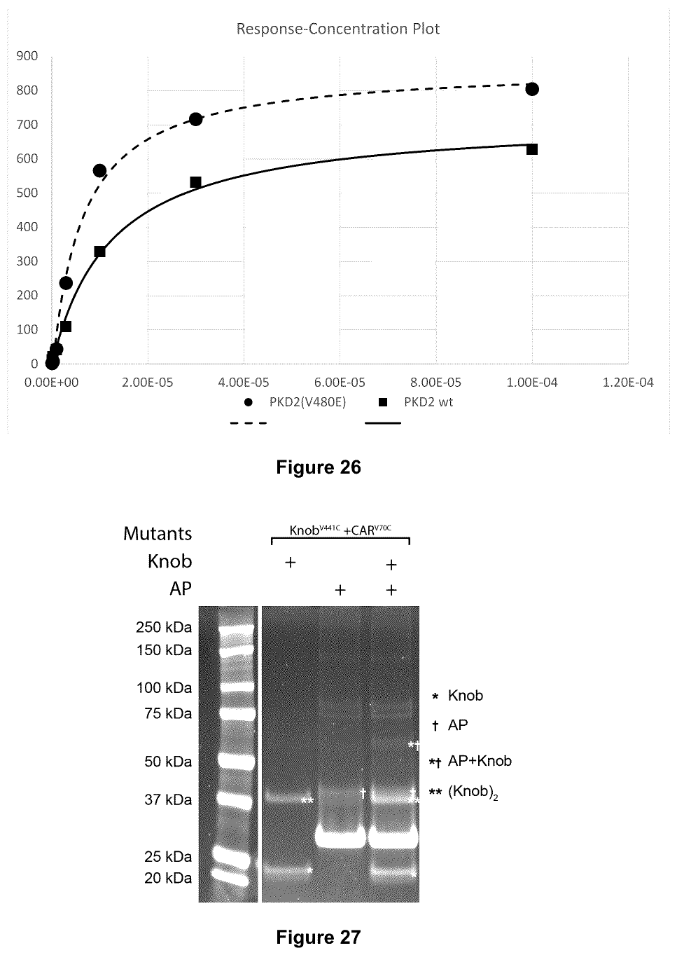

The PKD2(V480E) variant of AAVR has improved affinity for the AAV capsid, which makes it superior as an adapter protein (see FIG. 26).

The use of viral adaptor proteins to retarget viruses can be applied to adenovirus using CAR-fusion proteins as adaptors (see FIG. 27).

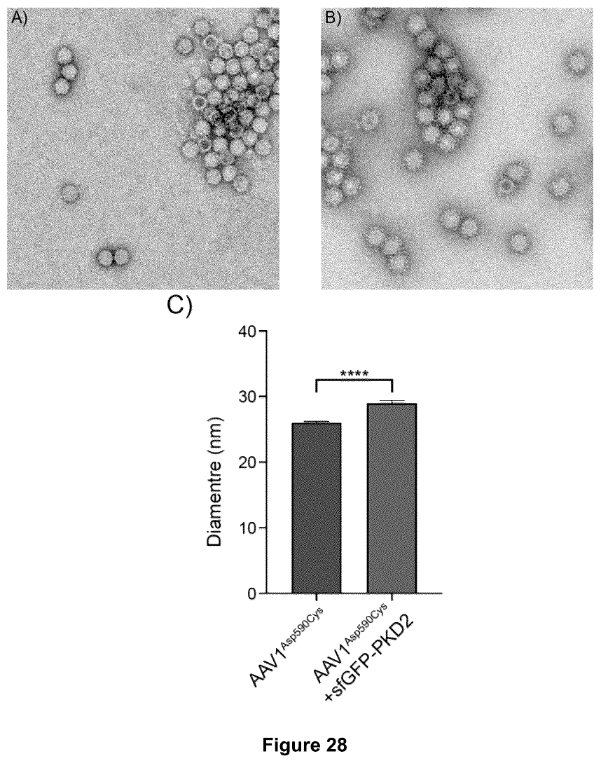

Complexes can be generated between AAV1(Asp590Cys) and an adaptor protein (see FIG. 28).

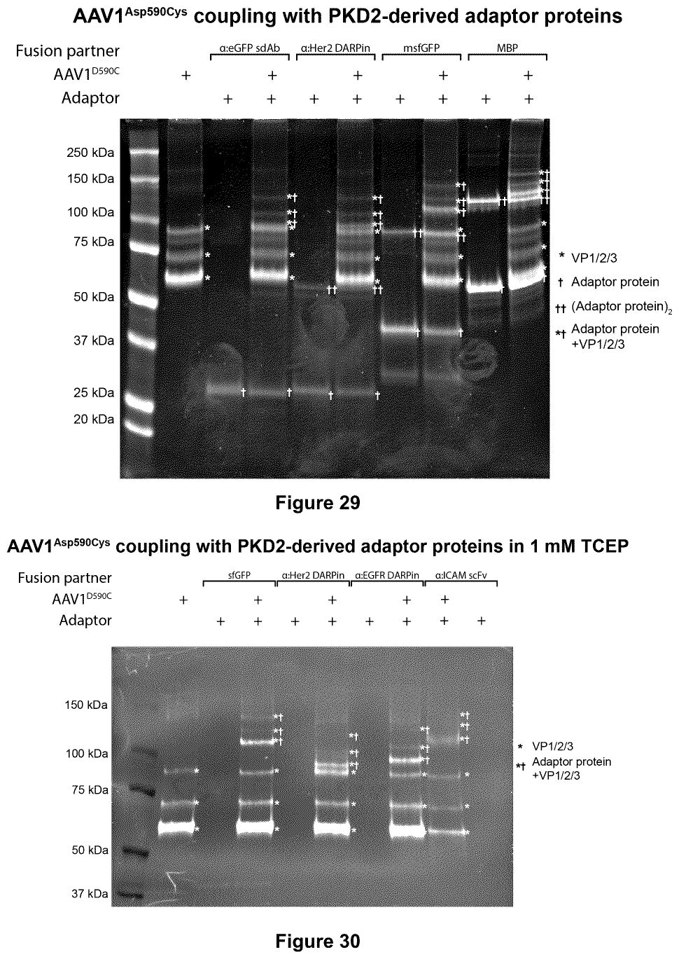

The identity of the fusion partner which composes the adaptor protein together with PKD2(Ser425Cys) can be customized in a modular fashion while maintaining the ability to form a covalent adduct. This implies that the present technology can be used to retarget the AAVs towards many different receptors by changing the identity of the fusion partner (see FIG. 29).

The covalently bound adducts between the adaptor protein and the viral capsid protein can be formed even in the presence of 1 mM of the reducing agent Tris(2-carboxyethyl)phosphine hydrochloride (TCEP), indicating that the disulfide bond between the proteins is formed at an interface which is not fully solvent exposed (see FIG. 30). Coupling in the presence of reducing agents such as TCEP may also be advantageous for coupling adapter proteins and viruses where the proteins and viruses may be partially oxidized prior to the coupling reaction.

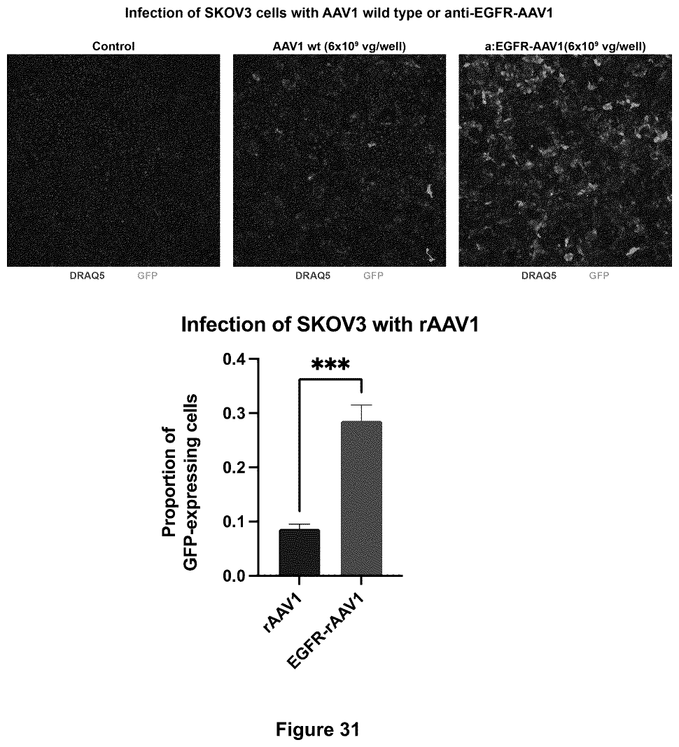

Re-targeting of AAV1(Asp590Cys) by means of an adaptor protein can substantially increase its infectivity (see FIG. 31).

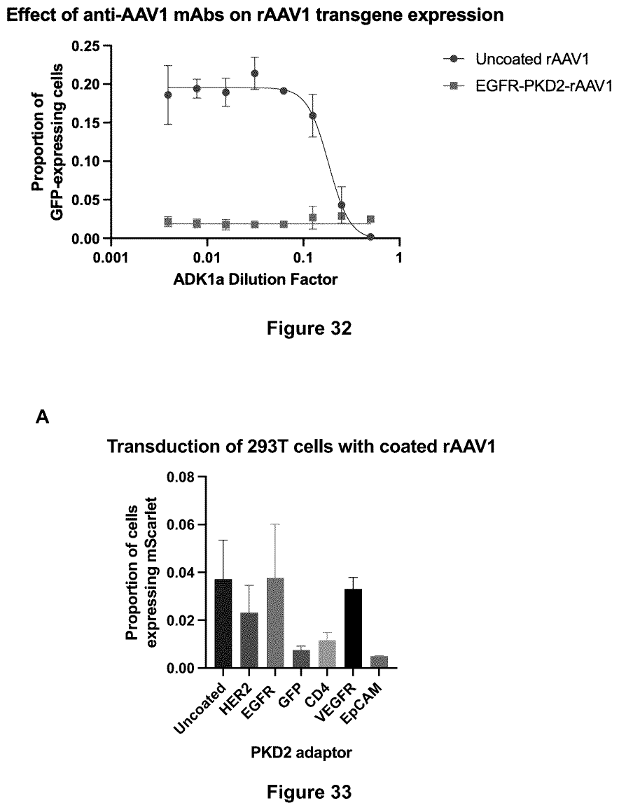

While recombinant AAV1 gets neutralized by the ADK1a antibody, the coated viral particles (CAR-V particles) maintain the same level of infectivity regardless of the concentration of neutralizing antibody and are hence resistant to neutralization (see FIG. 32).

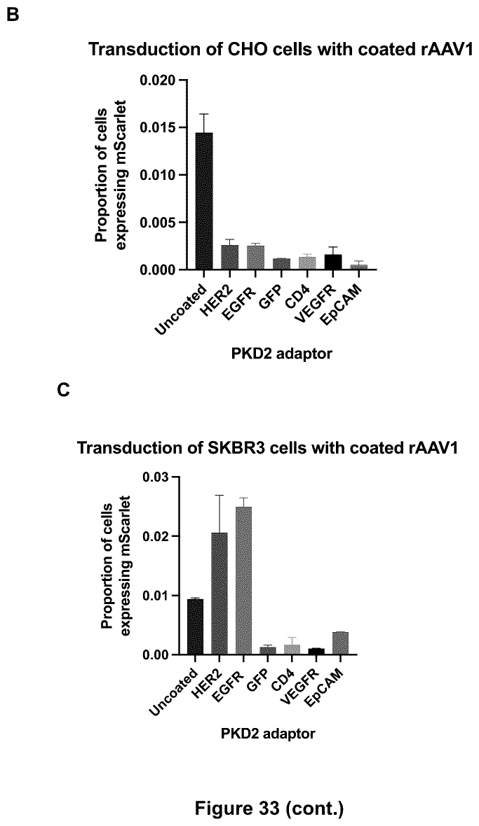

Coating AAV1(Asp590Cys) with adaptor proteins can enhance its specificity towards cell types expressing the receptor which interacts with the adaptor protein (see FIG. 33).

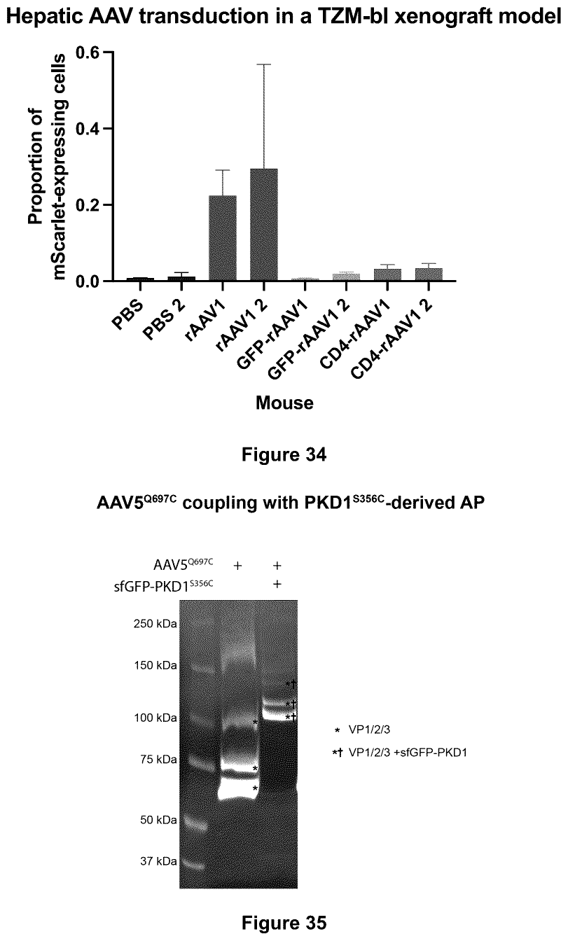

Coating of the virus with adaptor proteins abolishes infection of the liver, the main site of off-target infection for AAVs (see FIG. 34).

No appreciable bands corresponding to free VP proteins can be observed after incubation with the adaptor protein, indicating that the coupling reaches completion. This indicates that PKD1-derived adaptor proteins can be used to retarget AAV5^{Q697C} (see FIG. 35).

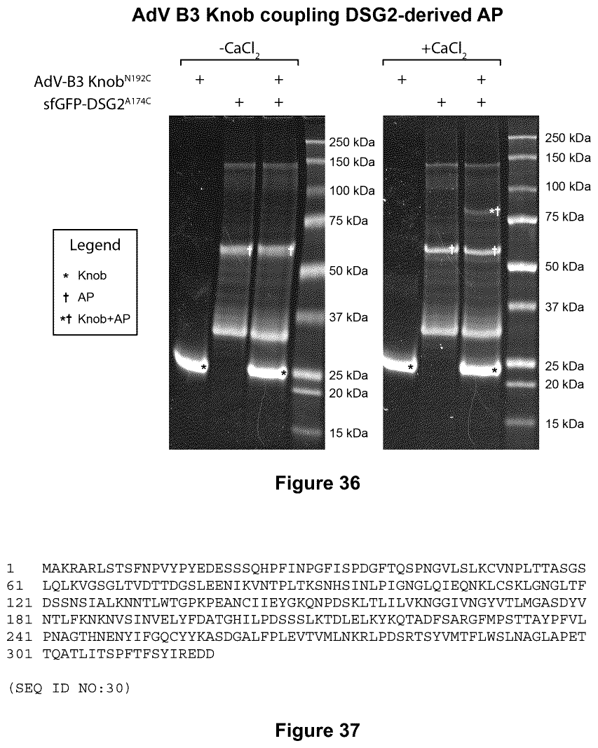

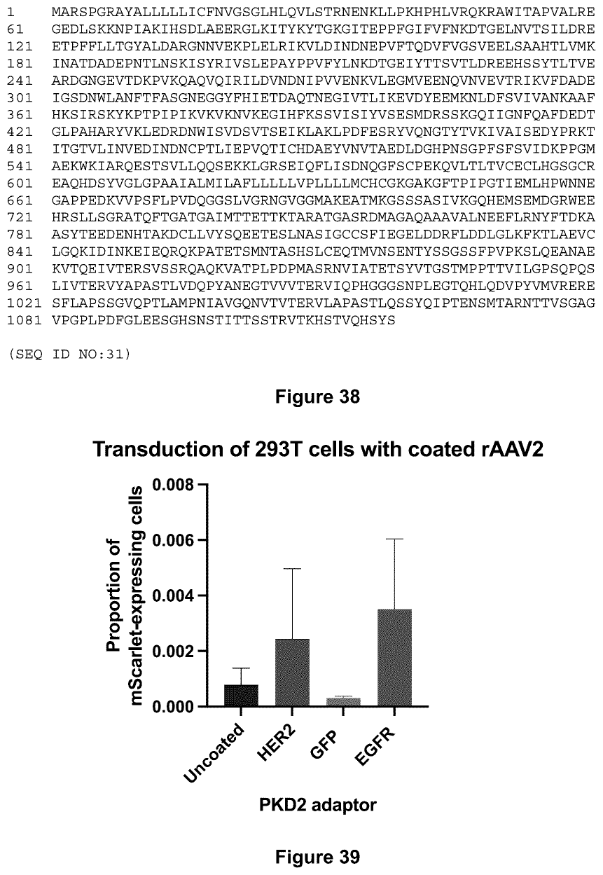

The formation of a covalent adduct between the fibre knob and the adaptor protein is observed exclusively in the presence of calcium ions, indicating that the formation of the bond is dependent on the Ca²⁺-dependent interaction between the knob and desmoglein2. Hence, DSG2-derived adaptor proteins can be covalently coupled to adenovirus B3 and used to redirect its tropism (see FIG. 36).

FIG. 39 shows that coating AAV2(Gln589Cys) with adaptor proteins can enhance its specificity towards cells expressing the receptor which interacts with the adaptor protein.

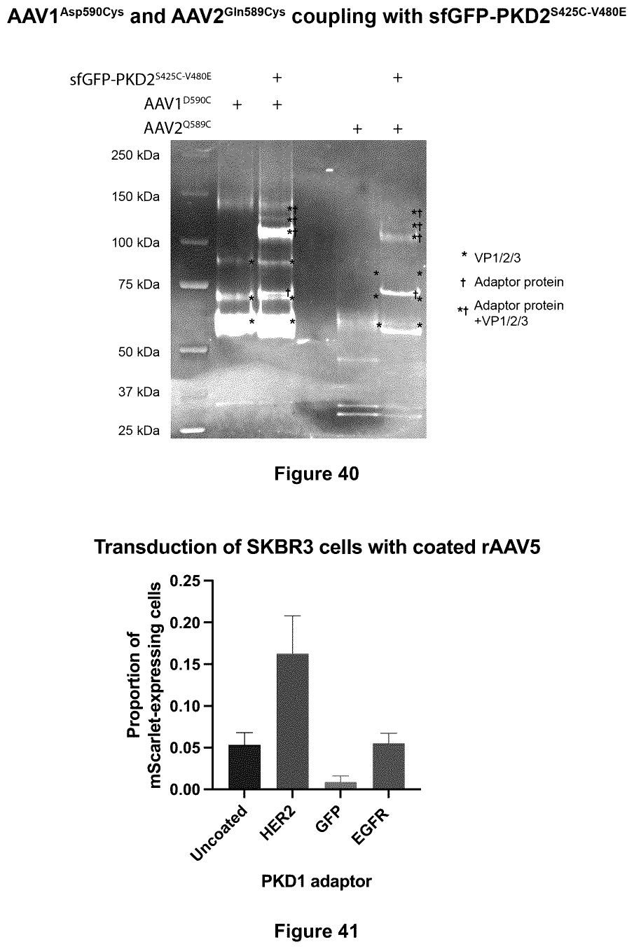

FIG. 40 shows the covalently bound adducts between the adaptor protein and the viral capsid proteins, as indicated by the shift in molecular weight of the capsid proteins in lanes 2 and 5 after the marker. Importantly, the adduct bands are formed for both the AAV1 and AAV2 mutant viruses. This confirms that PKD2-derived adaptor proteins can be used to redirect the tropism of AAV1 and AAV2.

FIG. 41 shows that coating AAV5(Gln697Cys) with adaptor proteins can permit retargeting by enhancing its specificity towards cells expressing the corresponding receptor.

These additional data demonstrate the general applicability of the principles described herein to multiple different viral adaptor proteins and multiple different virus types. In particular the present application provides evidence supporting a general effect across 5 distinct virus-adapter protein pairs:
PKD2(Ser425Cys-Val480Glu)+AAV1(Asp590Cys)
PKD2(Ser425Cys-Val480Glu)+AAV2(Gln589Cys)
PKD1(Ser356Cys)+AAV5(Gln697Cys)
DSG2(Ala174Cys)+AdV-B3(Asn192Cys)
CAR(Val70Cys)+AdV-5(Val441Cys)

It will be apparent to those skilled in the art that various modifications and variations can be made to the compositions and processes of this invention. Thus, it is intended that the present invention cover such modifications and variations, provided they come within the scope of the appended claims and their equivalents.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Further embodiments of the present invention are disclosed below:
1. An isolated polypeptide capable of binding to an adeno-associated virus (AAV) capsid.
2. An AAV adaptor molecule comprising (i) an isolated polypeptide capable of binding to an adeno associated virus (AAV) capsid and (ii) a ligand.
3. The isolated polypeptide or AAV adaptor molecule according to any one of embodiments 1 or 2 wherein the isolated polypeptide or AAV adaptor molecule is capable of binding to an unmodified, wildtype viral capsid.
4. The isolated polypeptide or AAV adaptor molecule according to any one of embodiments 1 to 3 wherein the isolated polypeptide or AAV adaptor molecule is capable of binding to a viral capsid having 5 or fewer (for example 5, 4, 3, 2 or 1) amino acid point mutations compared to a wildtype viral capsid.
5. The isolated polypeptide or AAV adaptor molecule according to any one of embodiments 1 to 4 wherein the isolated polypeptide or AAV adaptor molecule is capable of binding to a viral capsid having an amino acid insertion or deletion of no more than 5 amino acids (i.e. 1, 2, 3, 4 or 5 amino acids) relative to a wildtype viral capsid.
6. The isolated polypeptide or AAV adaptor molecule according to any one of embodiments 1 to 5 wherein the isolated polypeptide or AAV adaptor molecule is capable of binding to a viral capsid having no insertions or deletions relative to a wildtype viral capsid.
7. The isolated polypeptide or AAV adaptor molecule according to any one of embodiments 1 to 6, wherein the isolated polypeptide or AAV adaptor molecule comprises a portion of adeno-associated virus receptor (AAVR, KIAA0319L) capable of binding to an adeno associated virus capsid.
8. The isolated polypeptide or AAV adaptor molecule according to any one of embodiments 1 to 7, wherein the isolated polypeptide or AAV adaptor molecule comprises the full-length sequence of adeno-associated virus receptor (AAVR, KIAA0319L).
9. The isolated polypeptide or AAV adaptor molecule according to any one of embodiments 7 to 8, wherein the adeno-associated virus receptor (AAVR, KIAA0319L) sequence comprises the amino acid sequence of SEQ ID NO:1.
10. The isolated polypeptide or AAV adaptor molecule according to any one of embodiments 7 to 9, comprising one or more point mutations in the AAVR polypeptide, optionally wherein the one or more point mutations are defined by reference to SEQ ID NO:1.
11. The isolated polypeptide or AAV adaptor molecule according to any one of embodiments 7 to 10, comprising one or more point mutations in the AAVR polypeptide, optionally wherein the one or more point mutations are defined by reference to a sequence selected from the list consisting of: SEQ ID NO:2, SEQ ID NO:3, SEQ ID NO:4, SEQ ID NO:5 and SEQ ID NO:6.
12. The isolated polypeptide or AAV adaptor molecule according to any one of embodiments 7 to 10, comprising a V480E mutation in the PKD2 domain, optionally wherein the one or more point mutations are defined by reference to SEQ ID NO:1.
13. The isolated polypeptide or AAV adaptor molecule according to any one of embodiments 7 to 12, comprising the sequence of SEQ ID NO:1 with a V480E mutation.
14. The isolated polypeptide or AAV adaptor molecule according to any one of embodiments 7 to 11, comprising the sequence of SEQ ID NO:3 with a V480E mutation.
15. The isolated polypeptide or AAV adaptor molecule according to any one of embodiments 7 to 14, comprising a S425C mutation in the PKD2 domain, optionally wherein the one or more point mutations are defined by reference to SEQ ID NO:1.
16. The isolated polypeptide or AAV adaptor molecule according to any one of embodiments 7 to 14, comprising the sequence of SEQ ID NO:1 with a S425C mutation.
17. The isolated polypeptide or AAV adaptor molecule according to any one of embodiments 7 to 14, comprising the sequence of SEQ ID NO:3 with a S435C mutation.
18. The isolated polypeptide or AAV adaptor molecule according to any one of embodiments 7 to 17, comprising S425C and V480E mutations in the PKD2 domain, optionally wherein the one or more point mutations are defined by reference to SEQ ID NO:1 or SEQ ID NO:3.
19. The isolated polypeptide or AAV adaptor molecule according to any one of embodiments 1 to 6, wherein the isolated polypeptide or AAV adaptor molecule comprises a portion of a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof
20. The isolated polypeptide or AAV adaptor molecule according to any one of embodiments 1 to 6, wherein the isolated polypeptide or AAV adaptor molecule comprises a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof.
21. The isolated polypeptide or AAV adaptor molecule according to any one of embodiments 1 to 20, further comprising one or more of:
   a) one or more non-natural amino acids,
   b) one or more chemical moieties cross linked to the polypeptide,
   c) a biotin tag, for example biotinylated AAVR,
   d) a SpyTag peptide, for example AAVR-SpyTag; and/or
   e) a Protein A or Protein G polypeptide.
22. The isolated polypeptide or AAV adaptor molecule according to any one of embodiments 1 to 21, wherein the ligand is capable of binding to a cell surface molecule.
23. The isolated polypeptide or AAV adaptor molecule according to any one of embodiments 1 to 22, wherein the ligand is a human protein, such as an antibody or antigen binding fragment thereof.
24. The isolated polypeptide or AAV adaptor molecule according to any one of embodiments 1 to 23, wherein the ligand is one or more ligands selected from the list consisting of:
   Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Interleukin-17, Interleukin-18, Interleukin-19, Interleukin-20, Interleukin-21, Interleukin-22, Interleukin-23, Interleukin-24, Interleukin-25, Interleukin-26, Interleukin-27, Interleukin-28, Interleukin-29, Interleukin-30, Interleukin-31, Interleukin-32, Interleukin-33, Interleukin-34, Interleukin-35, Insulin, Transferrin, CD2, CD58, CD59, CD2, CD40L/CD154, CD5, CD72, CD5L, CD23, CD70, CD80, CD86, S100Ap, CD178, CD155, CD106, CSF1, CD166, FasL, CD242, CD252, TRAIL, RANKL, APRIL, CD257, CD272, CD273, CD274, CD275, PD-L1, PD-L2, Cas13 and Cas7-11, endothelin, leptin, vasopressin, CD10, CD31, CD119, apelin, elabela, adrenomedullin, a targeting domain from botulinum toxin, a neuropeptide, a cytokine or a small molecule.
25. The isolated polypeptide or AAV adaptor molecule according to any one of embodiments 1 to 24, wherein the ligand is a small molecule, optionally wherein the small molecule is linked to the isolated polypeptide or AAV adaptor molecule via N-Hydroxysuccinimide (NHS) or maleimide.
26. The isolated polypeptide or AAV adaptor molecule according to any one of embodiments 1 to 24, wherein the ligand is a small molecule, optionally wherein the isolated polypeptide or AAV adaptor molecule comprises a noncanonical amino acid that links the small molecule to the isolated polypeptide or AAV adaptor molecule.
27. The isolated polypeptide or AAV adaptor molecule according to any one of embodiments 1 to 26, wherein the ligand binds one or more cell surface molecules selected from the list consisting of:
   Her2, Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-8 receptor, Interleukin-9 receptor, Inter-leukin-10 receptor, Interleukin-11 receptor, Interleukin-12 receptor, Interleukin-13 receptor, Interleukin-15 receptor, Interleukin-18 receptor, Interleukin-20 receptor, Interleukin-21 receptor, Interleukin-22 receptor, Interleukin-23 receptor, Interleukin-27 receptor, Interleukin-28 receptor, Insulin receptor, Transferrin receptor, CD58, CD2, CD2, CD59, CD40, CD72, CD5, CD36, CD19, CD21, CD81, CD27, CD28, CTLA-4, CD85j, CD95, CD96, α4p1 integrin, CD115, CD6, CD178, LFA-1, TNFRSF4, DR4, DR5, RANK/CD265, TACI/CD267, CD267, CD268, CD269, HVEM, PD1/CD279, B7-1/CD80, CD278, CD4, CD8, CD19, NMDAR, AMPAR, mGluR5, DRD1, DRD2, Bmp4, GLP1R, leptin receptor, α5R5 integrin and glycoRNAs.
28. The isolated polypeptide or AAV adaptor molecule according to any one of embodiments 7 to 27, wherein the portion of adeno-associated virus receptor (AAVR, KIAA0319L) comprises at least a portion of the PKD1 domain, at least a portion of the PKD2 domain, at least a portion of the PKD3 domain, at least a portion of the PKD4 domain, at least a portion of the PKD5 domain, or any combination thereof.
29. The isolated polypeptide or AAV adaptor molecule according to any one of embodiments 7 to 28, wherein the portion of adeno-associated virus receptor (AAVR, KIAA0319L) comprises the full PKD1 domain, the full PKD2 domain, the full PKD3 domain, the full PKD4 domain, the full PKD5 domain, or any combination thereof.
30. The isolated polypeptide or AAV adaptor molecule according to any one of embodiments 7 to 29, wherein the portion of adeno-associated virus receptor (AAVR, KIAA0319L) comprises a sequence selected from the list consisting of: SEQ ID NO:2, SEQ ID NO:3, SEQ ID NO:4, SEQ ID NO:5 and SEQ ID NO:6.
31. The isolated polypeptide or AAV adaptor molecule according to any one of embodiments 7 to 30, wherein the portion of adeno-associated virus receptor (AAVR, KIAA0319L) comprises the full-length sequence of adeno-associated virus receptor (AAVR, KIAA0319L), optionally wherein the full sequence comprises SEQ ID NO:1.
32. An adeno-associated virus particle bound to at least one isolated polypeptide or AAV adaptor molecule according to any one of embodiments 1 to 31.
33. An adeno-associated virus particle bound to at least 2, 3, 4 or 5 isolated polypeptides or AAV adaptor molecules according to any one of embodiments 1 to 32.
34. The adeno-associated virus particle according to embodiment 33, wherein the adeno-associated virus particle has specificity for two or more different target cells.
35. The adeno-associated virus particle according to embodiment 33, wherein the adeno-associated virus particle has increased tropism for two or more different target cells.
36. The adeno-associated virus particle according to embodiment 34 or 35, wherein the two or more target cells express different cell surface molecules.
37. The adeno-associated virus particle according to any one of embodiments 34 to 36, wherein the at least 2, 3, 4 or 5 isolated polypeptides or AAV adaptor molecules specifically bind to the different cell surface molecules on the two or more target cells.
38. The adeno-associated virus particle according to embodiment 33, wherein the adeno-associated virus particle has specificity for a target cell expressing two or more different cell surface molecules.
39. The adeno-associated virus particle according to embodiment 33, wherein the adeno-associated virus particle has increased tropism for a target cell expressing two or more different cell surface molecules.
40. The adeno-associated virus particle according to embodiments 38 or 39, wherein the at least 2, 3, 4 or 5 isolated polypeptides or AAV adaptor molecules specifically bind to the two or more different cell surface molecules on the target cell.
41. The adeno-associated virus particle according to embodiment 32, wherein the adeno associated virus is selected from the list consisting of: AAV1, AAV2, AAV3, AAV4, AAV5, AAV6, AAV7, AAV8, AAV9, AAV10, AAV11, AAV12, AAV13, AAVrh.8, AAVrh.10, AAVrh.74.
42. The adeno-associated virus particle according to any one of embodiments 32 or 41, wherein the adeno-associated virus is AAV2.
43. The adeno-associated virus particle according to any one of embodiments 32 to 42, wherein the adeno-associated virus is a wildtype AAV.
44. The adeno-associated virus particle according to any one of embodiments 32 to 43, wherein the adeno-associated virus comprises a portion of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
45. The adeno-associated virus particle according to any one of embodiments 32 to 44, wherein the adeno-associated virus comprises the full-length of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
46. The adeno-associated virus particle according to any one of embodiments 32 to 45, wherein the adeno-associated virus has one or more conservative amino acid changes relative to the wildtype amino acid sequence, for example 1, 2, 3, 4, 5, 6, 7, 8, 9, 10, 11, 12, 13, 14, 15, 16, 17, 18, 19 or 20 conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
47. The adeno-associated virus particle according to any one of embodiments 32 to 46, wherein the adeno-associated virus has five or fewer non-conservative amino acid changes relative to the wildtype amino acid sequence, for example 5, 4, 3, 2 or 1 non-conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
48. The adeno-associated virus particle according to any one of embodiments 32 to 47, wherein the adeno-associated virus particle comprises one or more amino acid changes in the capsid protein that mediates the interaction with non-protein binders such as heparan sulfate proteoglycan (HSPG), O-linked sialic acid, N-linked sialic acid, N-linked galactose.
49. The adeno-associated virus particle according to embodiment 48, wherein the one or more mutations comprise arginine residues 585 and 588 of AAV2.
50. A pharmaceutical composition comprising an adeno-associated virus particle according to any one of embodiments 32 to 49.
51. A method of modifying an AAV particle comprising:
   providing an AAV particle;
   providing an isolated polypeptide capable of binding to an adeno-associated virus (AAV) capsid; and
   combining the AAV particle and the isolated polypeptide such that the isolated polypeptide binds to the AAV particle.
52. A method of modifying an AAV particle comprising:
   providing an AAV particle;
   providing an AAV adaptor molecule comprising an isolated polypeptide capable of binding to an adeno associated virus (AAV) capsid and a ligand; and
   combining the AAV particle and the AAV adaptor molecule such that the isolated fusion polypeptide binds to the AAV particle.
53. A method of modifying an AAV particle comprising:
   providing an AAV particle;
   providing two or more (e.g. 2, 3, 4 or 5) isolated polypeptides capable of binding to an adeno-associated virus (AAV) capsid; and
   combining the AAV particle and the isolated polypeptides such that the isolated polypeptides bind to the AAV particle.
54. A method of modifying an AAV particle comprising:
   providing an AAV particle;
   providing two or more (e.g. 2, 3, 4 or 5) AAV adaptor molecules comprising an isolated polypeptide capable of binding to an adeno associated virus (AAV) capsid and a ligand; and
   combining the AAV particle and the AAV adaptor molecules such that the AAV adaptor molecules bind to the AAV particle.
55. The method of modifying an AAV particle according to any one of embodiments 51 to 54, wherein the binding of the isolated polypeptide or AAV adaptor molecule to the AAV particle reduces or abolishes the natural tropism of one or more AAV capsid proteins.
56. The method of modifying an AAV particle according to any one of embodiments 51 to 55, wherein the binding of the isolated polypeptide or AAV adaptor molecule to the AAV particle increases the tropism of the AAV particle for one or more cell types.
57. The method of modifying an AAV particle according to any one of embodiments 51 to 56, wherein the binding of the isolated polypeptide or AAV adaptor molecule to the AAV particle increases the tropism of the AAV particle for one or more cell types selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.
58. The method of modifying an AAV particle according to any one of embodiments 51 to 57, wherein the AAV particle has reduced or increased tropism compared to the natural tropism of an AAV particle not comprising the isolated polypeptide or the AAV adaptor molecule of at least 10%, for example at least 20%, at least 30%, at least 40%, at least 50%, at least 60%, at least 70%, at least 80%, at least 90% or at least 100%.
59. The method of modifying an AAV particle according to any one of embodiments 51 to 58, wherein the AAV particle has specificity for two or more different target cells.
60. The method of modifying an AAV particle according to any one of embodiments 51 to 58, wherein the AAV particle has increased tropism for two or more different target cells.
61. The method of modifying an AAV particle according to embodiment 59 or 60, wherein the two or more target cells express different cell surface molecules.
62. The method of modifying an AAV particle according to any one of embodiments 59 to 61, wherein the at least 2, 3, 4 or 5 isolated polypeptides or AAV adaptor molecules specifically bind to the different cell surface molecules on the two or more target cells.
63. The method of modifying an AAV particle according to any one of embodiments 51 to 58, wherein the adeno-associated virus particle has specificity for a target cell expressing two or more different cell surface molecules.
64. The method of modifying an AAV particle according to any one of embodiments 51 to 58, wherein the adeno-associated virus particle has increased tropism for a target cell expressing two or more different cell surface molecules.
65. The method of modifying an AAV particle according to embodiment 63 or 64, wherein the at least 2, 3, 4 or 5 isolated polypeptides or AAV adaptor molecules specifically bind to the two or more different cell surface molecules on the target cell.
66. A method of targeting an AAV particle to a target cell comprising:
   providing an AAV particle;
   providing an AAV adaptor molecule comprising an isolated polypeptide capable of binding to an adeno associated virus (AAV) capsid and a ligand specific to the target cell;
   combining the AAV particle and the AAV adaptor molecule such that the AAV adaptor molecule binds to the AAV particle, thereby generating a modified AAV particle; and
   contacting a mixture of cells comprising the target cell with the modified AAV particle.
67. A method of targeting an AAV particle to a target cell comprising:
   providing an AAV particle;
   providing two or more (e.g. 2, 3, 4 or 5) AAV adaptor molecules comprising an isolated polypeptide capable of binding to an adeno associated virus (AAV) capsid and a ligand specific to the target cell;
   combining the AAV particle and two or more AAV adaptor molecules such that the AAV adaptor molecules bind to the AAV particle, thereby generating a modified AAV particle; and
   contacting a mixture of cells comprising the target cell with the modified AAV particle, wherein the target cell expresses two or more different cell surface molecules, optionally wherein the AAV adaptor molecules specifically bind to the two or more different cell surface molecules on the target cell.
68. A method of targeting an AAV particle to two or more target cells comprising: providing an AAV particle;
   providing two or more (e.g. 2, 3, 4 or 5) AAV adaptor molecules comprising an isolated polypeptide capable of binding to an adeno associated virus (AAV) capsid and a ligand specific to at least one of the two or more target cells;
   combining the AAV particle and two or more AAV adaptor molecules such that the AAV adaptor molecules bind to the AAV particle, thereby generating a modified AAV particle; and
   contacting a mixture of cells comprising the two or more target cells with the modified AAV particle, wherein the two or more target cells express two or more different cell surface molecules, optionally wherein the AAV adaptor molecules specifically bind to the different cell surface molecules on the two or more target cells.
69. The method of modifying an AAV particle or the method of targeting an AAV particle to a target cell according to any one of embodiments 51 to 68, wherein the isolated polypeptide capable of binding to an adeno associated virus (AAV) capsid comprises a portion of adeno-associated virus receptor (AAVR, KIAA0319L).
70. The method of modifying an AAV particle or the method of targeting an AAV particle to a target cell according to any one of embodiments 51 to 69, wherein the isolated polypeptide capable of binding to an adeno associated virus (AAV) capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination thereof.
71. The method of modifying an AAV particle or the method of targeting an AAV particle to a target cell according to any one of embodiments 51 to 70, wherein the isolated polypeptide capable of binding to an adeno associated virus (AAV) capsid comprises the full PKD1 domain, the full PKD2 domain, the full PKD3 domain, the full PKD4 domain, the full PKD5 domain of adeno-associated virus receptor (AAVR, KIAA0319L), or any combination thereof.
72. The method of modifying an AAV particle or the method of targeting an AAV particle to a target cell according to any one of embodiments 51 to 71, wherein the isolated polypeptide capable of binding to an adeno associated virus (AAV) capsid comprises one or more mutations in the AAVR polypeptide, optionally wherein the one or more mutations are defined by reference to SEQ ID NO:1.
73. The method of modifying an AAV particle or the method of targeting an AAV particle to a target cell according to any one of embodiments 51 to 72, wherein the isolated polypeptide capable of binding to an adeno associated virus (AAV) capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination thereof and wherein the isolated polypeptide capable of binding to an adeno associated virus (AAV) capsid comprises one or more mutations in the AAVR polypeptide, optionally wherein the one or more mutations are defined by reference to SEQ ID NO:1.
74. The method of modifying an AAV particle or the method of targeting an AAV particle to a target cell according to any one of embodiments 51 to 73, wherein the isolated polypeptide capable of binding to an adeno associated virus (AAV) capsid comprises the full PKD1 domain, the full PKD2 domain, the full PKD3 domain, the full PKD4 domain, the full PKD5 domain of adeno-associated virus receptor (AAVR, KIAA0319L), or any combination thereof and wherein the isolated polypeptide capable of binding to an adeno associated virus (AAV) capsid comprises one or more mutations in the AAVR polypeptide, optionally wherein the one or more mutations are defined by reference to SEQ ID NO:1.
75. The method of modifying an AAV particle or the method of targeting an AAV particle to a target cell according to any one of embodiments 51 to 74, wherein the isolated polypeptide capable of binding to an adeno associated virus (AAV) capsid comprises a sequence selected from the list consisting of: SEQ ID NO:2, SEQ ID NO:3, SEQ ID NO:4, SEQ ID NO:5 and SEQ ID NO:6.
76. The method of modifying an AAV particle or the method of targeting an AAV particle to a target cell according to any one of embodiments 51 to 75, wherein the isolated polypeptide capable of binding to an adeno associated virus (AAV) capsid comprises the full-length sequence of adeno-associated virus receptor (AAVR, KIAA0319L), optionally wherein the full sequence comprises SEQ ID NO:1.
77. The method of modifying an AAV particle or the method of targeting an AAV particle to a target cell according to any one of embodiments 51 to 76, wherein the isolated polypeptide capable of binding to an adeno associated virus (AAV) capsid comprises a portion of adeno-associated virus receptor having one or more point mutations, optionally wherein the one or more point mutations are defined by reference to SEQ ID NO:1.
78. The method of modifying an AAV particle or the method of targeting an AAV particle to a target cell according to any one of embodiments 51 to 76, wherein the isolated polypeptide capable of binding to an adeno associated virus (AAV) capsid comprises a portion of adeno-associated virus receptor having one or more point mutations, optionally wherein the one or more point mutations are defined by reference to a sequence selected from the list consisting of: SEQ ID NO:2, SEQ ID NO:3, SEQ ID NO:4, SEQ ID NO:5 and SEQ ID NO:6.
79. The method of modifying an AAV particle or the method of targeting an AAV particle to a target cell according to any one of embodiments 51 to 77, wherein the isolated polypeptide capable of binding to an adeno associated virus (AAV) capsid comprises a V480E mutation in the PKD2 domain, optionally wherein the one or more point mutations are defined by reference to SEQ ID NO:1.
80. The method of modifying an AAV particle or the method of targeting an AAV particle to a target cell according to any one of embodiments 51 to 79, wherein the isolated polypeptide capable of binding to an adeno associated virus (AAV) capsid comprises the sequence of SEQ ID NO:1 with a V480E mutation.
81. The method of modifying an AAV particle or the method of targeting an AAV particle to a target cell according to any one of embodiments 51 to 78, wherein the isolated polypeptide capable of binding to an adeno associated virus (AAV) capsid comprises the sequence of SEQ ID NO:3 with a V480E mutation.
82. The method of modifying an AAV particle or the method of targeting an AAV particle to a target cell according to any one of embodiments 51 to 81, wherein the isolated polypeptide capable of binding to an adeno associated virus (AAV) capsid comprises a S425C mutation in the PKD2 domain, optionally wherein the one or more point mutations are defined by reference to SEQ ID NO:1.
83. The method of modifying an AAV particle or the method of targeting an AAV particle to a target cell according to any one of embodiments 51 to 82, wherein the isolated polypeptide capable of binding to an adeno associated virus (AAV) capsid comprises the sequence of SEQ ID NO:1 with a S425C mutation.
84. The method of modifying an AAV particle or the method of targeting an AAV particle to a target cell according to any one of embodiments 51 to 83, wherein the isolated polypeptide capable of binding to an adeno associated virus (AAV) capsid comprises the sequence of SEQ ID NO:3 with a S425C mutation.
85. The method of modifying an AAV particle or the method of targeting an AAV particle to a target cell according to any one of embodiments 51 to 84, wherein the isolated polypeptide capable of binding to an adeno associated virus (AAV) capsid comprises S425C and V480E mutations in the PKD2 domain, optionally wherein the one or more point mutations are defined by reference to SEQ ID NO:1 or SEQ ID NO:3.
86. A method of delivering a nucleic acid sequence of interest to a target cell comprising contacting the target cell with the modified AAV particle according to any one of embodiments 32 to 49, or the pharmaceutical composition according to embodiment 50, wherein the AAV particle comprises the nucleic acid sequence of interest.
87. The method of embodiment 86, wherein the nucleic acid sequence of interest encodes a protein selected from the list consisting of:
   RAB escort protein 1, RPE65, Factor VIII, Factor IX, Cochlin, CLN7, acid a-glucosidase (GAA), Aquaporin 1, Glial cell line-derived neurotrophic factor, aspartoacylase, Aromatic L-amino Acid Decarboxylase, Defects in Retinitis Pigmentosa GTPase Regulator, sarcoplasmic reticulum calcium ATPase, Cyclic nucleotide gated channel beta 3, neurturin, Galactosidase beta 1, Glucose-6-Phosphatase, Phenylalanine hydroxylase, Ornithine Transcarbamylase, Dystrophin and Carnitine palmitoyltransferase II, phenylalanine hydroxylase (PAH), Cystic fibrosis transmembrane conductance regulator (CFTR).
88. The method according to any one of embodiments 66 to 87, wherein the target cell is in vitro.
89. The method according to any one of embodiments 66 to 87, wherein the target cell is in vivo in a subject.
90. The method according to anyone of embodiments 66 to 89, wherein the target cell is selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.
91. The method according to embodiment 89 or 90, wherein the subject is a human.
92. The method according to any one of embodiments 66 to 93, wherein the target cell is a human target cell.
93. An AAV particle according to any one of embodiments 32 to 49, or a pharmaceutical composition according to embodiment 50 for use as a medicament.
94. An AAV particle according to any one of embodiments 32 to 49, or a pharmaceutical composition according to embodiment 50 for use in a method of treating a disease in a subject in need thereof, the method comprising delivering a nucleic acid sequence of interest to a target cell by contacting the target cell with the AAV particle.
95. The AAV particle or pharmaceutical composition for use according to embodiment 94, wherein the disease is selected from the list consisting of:
   a neurodegenerative disorder, cancer, Duchenne muscular dystrophy, haemophilia, a congenital blindness disorder, diabetes, cystic fibrosis, choroideremia, hemophilia A, hemophilia B, CLN7 disease, Pompe disease, Parkinson's disease, Canavan disease, demyelinating diseases, inherited retinal dystrophy due to RPE65 mutations, aromatic L-amino acid decarboxylase (AADC) deficiency, X-linked retinitis pigmentosa, Leber congenital amaurosis, Churg-Strauss Syndrome (CSS), critical limb ischemia, achromatopsia, Alzheimer's disease, macular degeneration, ornithine transcarbamylase deficiency, Wilson disease, glycogen storage disease type IA, Crigler Najjar syndrome, Tay-Sachs disease, Sandhoff disease, multiple myeloma, multiple system atrophy, gangliosidosis, Danon disease, Fabry disease, Batten disease, phenylketonuria, rheumatoid arthritis, mucopolysaccharidosis type IIIa, Sanfilippo syndrome B, mucopolysaccharidosis type VI, alpha 1-antitrypsin deficiency, spinal muscular atrophy type 1, Krabbe disease, Becker muscular dystrophy, Charcot-Marie-Tooth neuropathy type 1a, carnitine palmitoyltransferase II (CPT II) deficiency and trimethylaminuria, cystic fibrosis, phenylketonuria.
96. An AAV particle according to any one of embodiments 32 to 49, or a pharmaceutical composition according to embodiment 50 for use in a method of treating a disease in a subject in need thereof, the method comprising delivering a nucleic acid sequence of interest to a target cell by contacting the target cell with the AAV particle, wherein the targeting of the specified target cell treats or ameliorates the specified disease according to the following list:
   a) neurons for treatment of neurodegenerative disorders;
   b) immune cells (e.g. macrophages, microglia, T-cells, B-cells, dendritic cells, antigen presenting cells, NK cells) for treatment of cancer;
   c) immune cells (e.g. macrophages, microglia, T-cells, B-cells, dendritic cells, antigen presenting cells, NK cells) to elicit an immune response;
   d) cancer or tumour cells for treatment of cancer;
   e) muscle cells for treatment of Duchenne muscular dystrophy;
   f) hepatocytes for treatment of haemophilia;
   g) photoreceptor cells for congenital blindness disorders;
   h) pancreatic beta cells for treatment of diabetes; or
   i) lung cells for treatment of cystic fibrosis.
97. An AAV particle or a pharmaceutical composition for use according to any one of embodiments 93 to 97 wherein the AAV particle or a pharmaceutical composition is administered to the subject via aerosol (e.g to lung cells), intramuscularly, intraarterially (e.g. via the hepatic artery), intraarticularly, subretinally, intracranially, intravenously, intrathecally. intracoronarily or subcutaneously.
98. An isolated polypeptide comprising one or more cysteine residues capable of covalently binding to a viral capsid.
99. A virus adaptor molecule comprising (i) an isolated polypeptide comprising one or more cysteine residues capable of covalently binding to a viral capsid and (ii) a ligand.
100. The isolated polypeptide according to embodiment 98 wherein the one or more cysteine residues capable of covalently binding to a viral capsid are heterologous to the isolated polypeptide.
101. The virus adaptor molecule according to embodiment 99 comprising (i) an isolated polypeptide comprising one or more cysteine residues capable of covalently binding to a viral capsid and (ii) a ligand, wherein the one or more cysteine residues are heterologous to the isolated polypeptide.
102. The isolated polypeptide or covalent binding virus adaptor molecule according to any one of embodiments 98 to 101 wherein the isolated polypeptide or covalent binding virus adaptor molecule is capable of binding to an unmodified, wildtype viral capsid.
103. The isolated polypeptide or covalent binding virus adaptor molecule according to any one of embodiments 98 to 102 wherein the isolated polypeptide or covalent binding virus adaptor molecule is capable of binding to a viral capsid having 5 or fewer (for example 5, 4, 3, 2 or 1) amino acid point mutations compared to a wildtype viral capsid.
104. The isolated polypeptide or covalent binding virus adaptor molecule according to any one of embodiments 98 to 103 wherein the isolated polypeptide or covalent binding virus adaptor molecule is capable of binding to a viral capsid having an amino acid insertion or deletion of no more than 5 amino acids (i.e. 1, 2, 3, 4 or 5 amino acids) relative to a wildtype viral capsid.
105. The isolated polypeptide or covalent binding virus adaptor molecule according to any one of embodiments 98 to 103 wherein the isolated polypeptide or covalent binding virus adaptor molecule is capable of binding to a viral capsid having no insertions or deletions relative to a wildtype viral capsid.
106. The isolated polypeptide or covalent binding virus adaptor molecule according to any one of embodiments 98 to 105, wherein the isolated polypeptide or virus adaptor molecule comprises a portion of adeno-associated virus receptor (AAVR, KIAA0319L) having one or more cysteine residues and capable of covalently binding to an adeno associated virus capsid.
107. The isolated polypeptide or covalent binding virus adaptor molecule according to any one of embodiments 98 to 106, wherein the isolated polypeptide or virus adaptor molecule comprises the full-length sequence of adeno-associated virus receptor (AAVR, KIAA0319L) having one or more cysteine residues and capable of covalently binding to an adeno associated virus capsid.
108. The isolated polypeptide or covalent binding virus adaptor molecule according to any one of embodiments 106 or 107, wherein the adeno-associated virus receptor (AAVR, KIAA0319L) sequence comprises the amino acid sequence of SEQ ID NO:1.
109. The isolated polypeptide or covalent binding virus adaptor molecule according to any one of embodiments 106 to 108, comprising one or more point mutations in the AAVR polypeptide, optionally wherein the one or more point mutations are defined by reference to SEQ ID NO:1.
110. The isolated polypeptide or covalent binding virus adaptor molecule according to any one of embodiments 106 to 109, comprising one or more point mutations in the AAVR polypeptide, optionally wherein the one or more point mutations are defined by reference to a sequence selected from the list consisting of: SEQ ID NO:2, SEQ ID NO:3, SEQ ID NO:4, SEQ ID NO:5 and SEQ ID NO:6.
111. The isolated polypeptide or covalent binding virus adaptor molecule according to any one of embodiments 106 to 110, comprising a V480E mutation in the PKD2 domain, optionally wherein the one or more point mutations are defined by reference to SEQ ID NO:1.
112. The isolated polypeptide or covalent binding virus adaptor molecule according to any one of embodiments 106 to 110, comprising the sequence of SEQ ID NO:1 with a V480E mutation.
113. The isolated polypeptide or covalent binding virus adaptor molecule according to any one of embodiments 106 to 110, comprising the sequence of SEQ ID NO:3 with a V480E mutation.
114. The isolated polypeptide or covalent binding virus adaptor molecule according to any one of embodiments 106 to 113, comprising a S425C mutation in the PKD2 domain, optionally wherein the one or more point mutations are defined by reference to SEQ ID NO:1.
115. The isolated polypeptide or covalent binding virus adaptor molecule according to any one of embodiments 106 to 114, comprising the sequence of SEQ ID NO:1 with a S425C mutation.
116. The isolated polypeptide or covalent binding virus adaptor molecule according to any one of embodiments 106 to 115, comprising the sequence of SEQ ID NO:3 with a S425C mutation.
117. The isolated polypeptide or covalent binding virus adaptor molecule according to any one of embodiments 106 to 116, comprising S425C and V480E mutations in the PKD2 domain, optionally wherein the one or more point mutations are defined by reference to SEQ ID NO:1 or SEQ ID NO:3.
118. The isolated polypeptide or covalent binding virus adaptor molecule according to any one of embodiments 106 to 117, comprising one or more point mutations in the AAVR polypeptide to introduce one or more heterologous cysteine residues, optionally wherein the one or more point mutations are defined by reference to SEQ ID NO:1.
119. The isolated polypeptide or covalent binding virus adaptor molecule according to any one of embodiments 106 to 118, comprising one or more point mutations in the AAVR polypeptide to introduce one or more heterologous cysteine residues, optionally wherein the one or more point mutations are defined by reference to a sequence selected from the list consisting of: SEQ ID NO:2, SEQ ID NO:3, SEQ ID NO:4, SEQ ID NO:5 and SEQ ID NO:6.
120. The isolated polypeptide or covalent binding virus adaptor molecule according to any one of embodiments 98 to 105, wherein the isolated polypeptide or covalent binding virus adaptor molecule comprises a portion of a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof. In some embodiments the isolated polypeptide or AAV adaptor molecule comprises an antibody or antigen binding fragment thereof.
121. The isolated polypeptide or covalent binding virus adaptor molecule according to any one of embodiments 98 to 105, wherein the isolated polypeptide or virus adaptor molecule comprises a polypeptide selected from the list consisting of: a DARPin, a nanobody, a suitable antibody-like protein, a protein derived from the lipocalin fold, an affibody (for example an affibody derived from the Z domain of Protein A), a domain of fibronectin, an SH3 domain of Fyn, an affimer or a scaffold derived from the protease inhibitor stefin A, a non-antibody scaffold protein (Adhiron) and an antibody or antigen binding fragment thereof. In some embodiments the isolated polypeptide or AAV adaptor molecule comprises an antibody or antigen binding fragment thereof.
122. The isolated polypeptide or covalent binding virus adaptor molecule according to any one of embodiments 98 to 105, 120 or 121, wherein the isolated polypeptide or covalent binding virus adaptor molecule comprises a portion of a coxsackievirus-adenovirus receptor (CXADR), optionally wherein the CXADR sequence is SEQ ID NO:20.
123. The isolated polypeptide or covalent binding virus adaptor molecule according to any one of embodiments 98 to 105, 120 or 121, wherein the isolated polypeptide or covalent binding virus adaptor molecule comprises the full-length sequence of a coxsackievirus-adenovirus receptor (CXADR), optionally wherein the CXADR sequence is SEQ ID NO:20.
124. The isolated polypeptide or covalent binding virus adaptor molecule according to any one of embodiments 98 to 105, 120 or 121, wherein the isolated polypeptide or covalent binding virus adaptor molecule comprises an antigen binding portion of neutralising antibody A20.
125. The isolated polypeptide or covalent binding virus adaptor molecule according to embodiment 124, wherein the isolated polypeptide or covalent binding virus adaptor molecule comprises a sequence substantially identical to one or more complementarity determining regions (CDRs) of neutralising antibody A20, for example wherein the one or more complementarity determining regions (CDRs) of neutralising antibody A20 may comprise 1, 2 or 3 amino acid point mutations, optionally wherein one or more of the point mutations is a heterologous cysteine residue.
126. The isolated polypeptide or covalent binding virus adaptor molecule according to embodiment 125, wherein the isolated polypeptide or covalent binding virus adaptor molecule comprises one or more sequences that are substantially identical to each of the complementarity determining regions (CDRs) of neutralising antibody A20, for example wherein 1, 2, 3, 4, 5 or 6 of the complementarity determining regions (CDRs) of neutralising antibody A20 may comprise 1, 2 or 3 amino acid point mutations, optionally wherein one or more of the point mutations is a heterologous cysteine residue.
127. The isolated polypeptide or covalent binding virus adaptor molecule according to embodiment 124, wherein the isolated polypeptide or covalent binding virus adaptor molecule comprises the full-length sequence of one or more complementarity determining regions (CDRs) of neutralising antibody A20, for example wherein the polypeptide comprises one or more of VHCDR1 of antibody A20 (SEQ ID NO:26), VHCDR2 of antibody A20 (SEQ ID NO:27), VHCDR3 of antibody A20 (SEQ ID NO:28), VLCDR1 of antibody A20 (SEQ ID NO:22), VLCDR2 of antibody A20 (SEQ ID NO:23) and VLCDR3 of antibody A20 (SEQ ID NO:24).
128. The isolated polypeptide or covalent binding virus adaptor molecule according to embodiment 127, wherein the isolated polypeptide or covalent binding virus adaptor molecule comprises all of the complementarity determining regions (CDRs) of neutralising antibody A20.
129. The isolated polypeptide comprising one or more cysteine residues according to any one of embodiments 98 to 105 or 120 to 128 wherein the polypeptide or covalent binding virus adaptor molecule comprises one or more sequences that are substantially identical to the full-length VL chain (SEQ ID NO:21) and VH chain (SEQ ID NO:25) sequences of neutralising antibody A20, for example wherein the VL and/or VH sequences of neutralising antibody A20 may comprise 1, 2, 3, 4, 5, 6, 7, 8, 9 or 10 amino acid point mutations, optionally wherein one or more of the point mutations is a heterologous cysteine residue.
130. The isolated polypeptide comprising one or more cysteine residues according to any one of embodiments 98 to 105 or 120 to 128 wherein the polypeptide or covalent binding virus adaptor molecule comprises the full-length VL chain (SEQ ID NO:21) and VH chain (SEQ ID NO:25) sequences of neutralising antibody A20.
131. The isolated polypeptide or covalent binding virus adaptor molecule according to any one of embodiments 98 to 130, further comprising one or more of:
   a) one or more non-natural amino acids,
   b) one or more chemical moieties cross linked to the polypeptide,
   c) a biotin tag,
   d) a SpyTag peptide, and/or
   e) a Protein A or Protein G polypeptide.
132. The isolated polypeptide or covalent binding virus adaptor molecule according to any one of embodiments 98 to 131, wherein the ligand is capable of binding to a cell surface molecule.
133. The isolated polypeptide or covalent binding virus adaptor molecule according to any one of embodiments 98 to 132, wherein the ligand is a human protein, such as an antibody or antigen binding fragment thereof.
134. The isolated polypeptide or covalent binding virus adaptor molecule according to any one of embodiments 98 to 133, wherein the ligand is one or more ligands selected from the list consisting of:
   Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Interleukin-17, Interleukin-18, Interleukin-19, Interleukin-20, Interleukin-21, Interleukin-22, Interleukin-23, Interleukin-24, Interleukin-25, Interleukin-26, Interleukin-27, Interleukin-28, Interleukin-29, Interleukin-30, Interleukin-31, Interleukin-32, Interleukin-33, Interleukin-34, Interleukin-35, Insulin, Transferrin, CD2, CD58, CD59, CD2, CD40L/CD154, CD5, CD72, CD5L, CD23, CD70, CD80, CD86, S100Ap, CD178, CD155, CD106, CSF1, CD166, FasL, CD242, CD252, TRAIL, RANKL, APRIL, CD257, CD272, CD273, CD274, CD275, PD-L1, PD-L2, Cas13 and Cas7-11, endothelin, leptin, vasopressin, CD10, CD31, CD119, apelin, elabela, adrenomedullin, a targeting domain from botulinum toxin, a neuropeptide, a cytokine or a small molecule.
135. The isolated polypeptide or covalent binding virus adaptor molecule according to any one of embodiments 98 to 134, wherein the ligand is a small molecule, optionally wherein the small molecule is linked to the isolated polypeptide or virus adaptor molecule via N-Hydroxysuccinimide (NHS) or maleimide.
136. The isolated polypeptide or covalent binding virus adaptor molecule according to any one of embodiments 98 to 134, wherein the ligand is a small molecule, optionally wherein the isolated polypeptide or virus adaptor molecule comprises a noncanonical amino acid that links the small molecule to the isolated polypeptide or virus adaptor molecule.
137. The isolated polypeptide or covalent binding virus adaptor molecule according to any one of embodiments 98 to 136, wherein the ligand binds one or more cell surface molecules selected from the list consisting of:
   Her2, Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-8 receptor, Interleukin-9 receptor, Inter-leukin-10 receptor, Interleukin-11 receptor, Interleukin-12 receptor, Interleukin-13 receptor, Interleukin-15 receptor, Interleukin-18 receptor, Interleukin-20 receptor, Interleukin-21 receptor, Interleukin-22 receptor, Interleukin-23 receptor, Interleukin-27 receptor, Interleukin-28 receptor, Insulin receptor, Transferrin receptor, CD58, CD2, CD2, CD59, CD40, CD72, CD5, CD36, CD19, CD21, CD81, CD27, CD28, CTLA-4, CD85j, CD95, CD96, α4p1 integrin, CD115, CD6, CD178, LFA-1, TNFRSF4, DR4, DR5, RANK/CD265, TACI/CD267, CD267, CD268, CD269, HVEM, PD1/CD279, B7-1/CD80, CD278, CD4, CD8, CD19, NMDAR, AMPAR, mGluR5, DRD1, DRD2, Bmp4, GLP1R, leptin receptor, α5β5 integrin and glycoRNAs.
138. The isolated polypeptide or covalent binding virus adaptor molecule according to any one of embodiments 106 to 137, wherein the portion of adeno-associated virus receptor (AAVR, KIAA0319L) comprises at least a portion of the PKD1 domain, at least a portion of the PKD2 domain, at least a portion of the PKD3 domain, at least a portion of the PKD4 domain, at least a portion of the PKD5 domain, or any combination thereof.
139. The isolated polypeptide or covalent binding virus adaptor molecule according to any one of embodiments 106 to 137, wherein the portion of adeno-associated virus receptor (AAVR, KIAA0319L) comprises the PKD1 domain, the PKD2 domain, the PKD3 domain, the PKD4 domain, the PKD5 domain, or any combination thereof.
140. The isolated polypeptide or covalent binding virus adaptor molecule according to any one of embodiments 106 to 138, wherein the portion of adeno-associated virus receptor (AAVR, KIAA0319L) comprises a sequence selected from the list consisting of: SEQ ID NO:2, SEQ ID NO:3, SEQ ID NO:4, SEQ ID NO:5 and SEQ ID NO:6.
141. The isolated polypeptide or covalent binding virus adaptor molecule according to any one of embodiments 106 to 140, wherein the portion of adeno-associated virus receptor (AAVR, KIAA0319L) comprises the full-length sequence of adeno-associated virus receptor (AAVR, KIAA0319L), optionally wherein the full sequence comprises SEQ ID NO:1.
142. A virus particle covalently bound to at least one isolated polypeptide or virus adaptor molecule according to any one of embodiments 98 to 141.
143. A virus particle covalently bound to at least 2, 3, 4 or 5 isolated polypeptides or virus adaptor molecules according to any one of embodiments 98 to 141.
144. The virus particle according to embodiment 143, wherein the virus particle has specificity for two or more different target cells.
145. The virus particle according to embodiment 143, wherein the virus particle has increased tropism for two or more different target cells.
146. The virus particle according to embodiment 144 or 145, wherein the two or more target cells express different cell surface molecules.
147. The virus particle according to any one of embodiments 144 to 146, wherein the at least 2, 3, 4 or 5 isolated polypeptides or virus adaptor molecules specifically bind to the different cell surface molecules on the two or more target cells.
148. The virus particle according to embodiment 143, wherein the virus particle has specificity for a target cell expressing two or more different cell surface molecules.
149. The virus particle according to embodiment 143, wherein the virus particle has increased tropism for a target cell expressing two or more different cell surface molecules.
150. The virus particle according to embodiments 148 or 149, wherein the at least 2, 3, 4 or 5 isolated polypeptides or virus adaptor molecules specifically bind to the two or more different cell surface molecules on the target cell.
151. The virus particle according to any one of embodiments 142 to 150 wherein one or more viral capsid proteins comprise one or more cysteine residues capable of covalently binding to an isolated polypeptide.
152. The virus particle according to any one of embodiments 142 to 151 wherein one or more viral capsid proteins comprise one or more cysteine residues capable of forming a disulphide bridge with one or more cysteine residues in an isolated polypeptide.
153. The virus particle according to any one of embodiments 142 to 152, wherein the virus is an adeno-associated virus, optionally wherein the adeno-associated virus is selected from the list consisting of: AAV1, AAV2, AAV3, AAV4, AAV5, AAV6, AAV7, AAV8, AAV9, AAV10, AAV11, AAV12, AAV13, AAVrh.8, AAVrh.10, AAVrh.74.
154. The virus particle according to any one of embodiments 142 to 153, wherein the adeno-associated virus is AAV2.
155. The virus particle according to any one of embodiments 142 to 154, wherein the adeno-associated virus is a wildtype AAV.
156. The virus particle according to any one of embodiments 142 to 155, wherein the adeno-associated virus comprises a portion of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
157. The virus particle according to any one of embodiments 142 to 156, wherein the adeno-associated virus comprises the full-length of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
158. The virus particle according to any one of embodiments 142 to 155, wherein the adeno-associated virus has one or more conservative amino acid changes relative to the wildtype amino acid sequence, for example 1, 2, 3, 4, 5, 6, 7, 8, 9, 10, 11, 12, 13, 14, 15, 16, 17, 18, 19 or 20 conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
159. The virus particle according to any one of embodiments 142 to 158, wherein the adeno-associated virus has five or fewer non-conservative amino acid changes relative to the wildtype amino acid sequence, for example 5, 4, 3, 2 or 1 non-conservative amino acid changes relative to the wildtype amino acid sequence, optionally wherein the wildtype amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
160. The virus particle according to any one of embodiments 142 to 159, wherein the virus is selected from the list consisting of: Adenovirus, Aichi virus, Australian bat lyssavirus, BK polyomavirus, Banna virus, Barmah forest virus, Bunyamwera virus, Bunyavirus, La CrosseBunyavirus, snowshoe hare Cercopithecine herpesvirus, Chandipura virus, Chikungunya virus, Cosavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus, C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human herpesvirus, Human immunodeficiency virus, Human papillomavirus, Human papillomavirus, Human parainfluenza, Human parvovirus, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephali-tis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New Jersey polyomavirus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus, ARoss river virus, Rotavirus, ARotavirus, BRotavirus, CRubella virus, Sagiyama virus, Salivirus, ASandfly fever sicilian virus, Sapporo virus, SARS coronavirus, Semliki forest virus, Seoul virus, Simian foamy virus, Simian virus 40, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus and Zika virus.
161. The virus particle according to embodiment 160, wherein the virus is a wildtype virus.
162. The virus particle according to embodiment 160, wherein the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a portion of CD46, optionally wherein the CD46 sequence is SEQ ID NO:29.
163. The virus particle according to embodiment 162, wherein the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a full-length sequence of CD46, optionally wherein the CD46 sequence is SEQ ID NO:29.
164. The virus particle according to embodiment 160, wherein the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a portion of the coxsackievirus-adenovirus receptor (CXADR), optionally wherein the CXADR sequence is SEQ ID NO:20.
165. The virus particle according to embodiment 164, wherein the virus is an adenovirus and the isolated polypeptide comprising one or more cysteine residues capable of covalently binding to a viral capsid or the virus adaptor molecule comprising an isolated polypeptide comprising one or more cysteine residues capable of covalently binding to a viral capsid and a ligand comprises a full-length sequence of a coxsackievirus-adenovirus receptor (CXADR), optionally wherein the CXADR sequence is SEQ ID NO:20.
166. The virus particle according to any one of embodiments 142 to 165, wherein one or more virus capsids have one or more heterologous cysteine residues introduced relative to the wildtype amino acid sequence, for example 1, 2, 3, 4, 5, 6, 7, 8, 9 or 10 heterologous cysteine residues relative to the wildtype amino acid sequence.
167. The virus particle according to embodiment 166, wherein the one or more heterologous cysteine residues are introduced at the interface between the isolated polypeptide or virus adaptor molecule and the virus capsid.
168. The virus particle according to embodiment 166 or 167, wherein the virus is AAV1 and the isolated polypeptide or an isolated fusion polypeptide comprises a portion of adeno-associated virus receptor (AAVR, KIAA0319L) capable of covalently binding to an adeno associated virus capsid, and
   wherein the AAV1 comprises one or more mutations selected from Gly266Cys, Thr504Cys and Asp590Cys, and
   wherein the AAVR polypeptide or fusion polypeptide comprises one or more mutations selected from Thr434Cys, Asp429Cys and Ser425Cys.
169. The virus particle according to embodiment 166 or 167, wherein the virus is AAV2 and the isolated polypeptide or an isolated fusion polypeptide comprises a portion of adeno-associated virus receptor (AAVR, KIAA0319L) capable of covalently binding to an adeno associated virus capsid, and
   wherein the AAV2 comprises one or more mutations selected from Gly265Cys, Thr503Cys and Gln589Cys, and
   wherein the AAVR polypeptide or fusion polypeptide comprises one or more mutations selected from Thr434Cys, Asp429Cys and Ser425Cys.
170. The virus particle according to embodiment 166 or 167, wherein the virus is AAV2 and the isolated polypeptide or an isolated fusion polypeptide comprises an antigen binding portion of neutralising antibody A20, and
   wherein the AAV2 comprises one or more mutations selected from Ser264Cys, Val708Cys and Asn717Cys, and
   wherein the neutralising antibody A20 polypeptides or fusion polypeptides comprise one or more mutations selected from VH Tyr102Cys, VH Ser56Cys and VL Ile93Cys.
171. The virus particle according to embodiment 166 or 167, wherein the virus is hAdV-11p and the isolated polypeptide or an isolated fusion polypeptide comprises a portion of hCD46, and
   wherein the hAdV-11p comprises the mutation Asn283Cys, and
   wherein the hCD46 comprises the mutation Thr42Cys.
172. The virus particle according to any one of embodiments 142 to 171, wherein the adeno-associated virus particle comprises one or more amino acid changes in the capsid protein that mediates the interaction with non-protein binders such as heparan sulfate proteoglycan (HSPG), O-linked sialic acid, N-linked sialic acid, N-linked galactose.
173. A pharmaceutical composition comprising a virus particle according to any one of embodiments 142 to 172.
174. A method of covalently modifying a virus particle comprising:
   providing a virus particle;
   providing an isolated polypeptide capable of covalently binding to a virus capsid of the virus particle; and
   combining the virus particle and the isolated polypeptide such that the isolated polypeptide covalently binds to the virus particle.
175. A method of covalently modifying a virus particle comprising:
   providing a virus particle;
   providing a virus adaptor molecule comprising an isolated polypeptide capable of covalently binding to a virus capsid of the virus particle and a ligand; and
   combining the virus particle and the virus adaptor molecule such that the virus adaptor molecule covalently binds to the virus particle.
176. A method of covalently modifying a virus particle comprising:
   providing a virus particle;
   providing two or more (e.g. 2, 3, 4 or 5) isolated polypeptides capable of covalently binding to a virus capsid of the virus particle; and
   combining the virus particle and the two or more isolated polypeptides such that the isolated polypeptides covalently bind to the virus particle.
177. A method of covalently modifying a virus particle comprising:
   providing a virus particle;
   providing two or more (e.g. 2, 3, 4 or 5) virus adaptor molecules comprising an isolated polypeptide capable of covalently binding to a virus capsid of the virus particle and a ligand; and
   combining the virus particle and the two or more virus adaptor molecules such that the virus adaptor molecules covalently bind to the virus particle.
178. The method of covalently modifying a virus particle according to any one of embodiments 174 to 177, wherein the method further comprises introducing one or more heterologous cysteine residues to the isolated polypeptide or virus adaptor molecule, for example 1, 2 or 3 heterologous cysteine residues.
179. The method of covalently modifying a virus particle according to any one of embodiments 174 to 178, wherein the method further comprises introducing one or more heterologous cysteine residues to the virus capsid, for example 1, 2 or 3 heterologous cysteine residues.
180. The method of covalently modifying a virus particle according to any one of embodiments 174 to 179, wherein the method further comprises introducing one or more heterologous cysteine residues to both the isolated polypeptide or virus adaptor molecule and to the virus capsid, for example 1, 2 or 3 heterologous cysteine residues to both the isolated polypeptide or virus adaptor molecule and to the virus capsid.
181. The method of covalently modifying a virus particle according to any one of embodiments 174 to 180, wherein the covalent binding of the isolated polypeptide or virus adaptor molecule to the virus particle reduces or abolishes the natural tropism of one or more virus capsid proteins.
182. The method of covalently modifying a virus particle according to any one of embodiments 174 to 180, wherein the covalent binding of the isolated polypeptide or virus adaptor molecule to the virus particle increases the tropism of the virus particle for one or more cell types.
183. The method of covalently modifying a virus particle according to any one of embodiments 174 to 182, wherein the AAV particle has specificity for two or more different target cells.
184. The method of covalently modifying a virus particle according to any one of embodiments 174 to 182, wherein the covalent binding of the isolated polypeptide or virus adaptor molecule to the virus particle increases the tropism of the virus particle for two or more different target cells.
185. The method of covalently modifying a virus particle according to embodiment 183 or 184, wherein the two or more target cells express different cell surface molecules.
186. The method of covalently modifying a virus particle according to any one of embodiments 183 to 185, wherein the at least 2, 3, 4 or 5 isolated polypeptides or virus adaptor molecules specifically bind to the different cell surface molecules on the two or more target cells.
187. The method of covalently modifying a virus particle according to any one of embodiments 174 to 184, wherein the virus particle has specificity for a target cell expressing two or more different cell surface molecules.
188. The method of covalently modifying a virus particle according to any one of embodiments 174 to 184, wherein the virus particle has increased tropism for a target cell expressing two or more different cell surface molecules.
189. The method of covalently modifying a virus particle according to embodiment 187 or 188, wherein the at least 2, 3, 4 or 5 isolated polypeptides or virus adaptor molecules specifically bind to the two or more different cell surface molecules on the target cell.
190. The method of covalently modifying a virus particle according to any one of embodiments 174 to 189, wherein the covalent binding of the isolated polypeptide or virus adaptor molecule to the virus particle increases and/or reduces the tropism of the virus particle for one or more cell types selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.
191. The method of covalently modifying a virus particle according to any one of embodiments 174 to 190, wherein the virus particle has reduced or increased tropism compared to the natural tropism of a virus particle not comprising the isolated polypeptide or the virus adaptor molecule of at least 10%, for example at least 20%, at least 30%, at least 40%, at least 50%, at least 60%, at least 70%, at least 80%, at least 90% or at least 100%.
192. The method of covalently modifying a virus particle according to any one of embodiments 174 to 191, wherein the covalent binding of the isolated polypeptide or virus adaptor molecule to the virus particle allows the virus particle to display protein antigens in a multimeric fashion using the virus as a scaffold.
193. The method of covalently modifying a virus particle according to any one of embodiments 174 to 192, wherein the covalent binding of the isolated polypeptide or virus adaptor molecule to the virus particle selectively induces cell lysis, optionally wherein cell lysis in induced in one or more cell types selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.
194. A method of targeting a virus particle to a target cell comprising:
   providing a virus particle;
   providing a virus adaptor molecule comprising an isolated polypeptide capable of covalently binding to a virus capsid of the virus particle and a ligand specific to the target cell;
   combining the virus particle and the virus adaptor molecule such that the virus adaptor molecule covalently binds to the virus particle, thereby generating a modified virus particle; and
   contacting a mixture of cells comprising the target cell with the modified virus particle.
195. A method of targeting a virus particle to a target cell comprising:
   providing a virus particle;
   providing two or more (e.g. 2, 3, 4 or 5) virus adaptor molecules comprising an isolated polypeptide capable of covalently binding to a virus capsid of the virus particle and a ligand specific to the target cell;
   combining the virus particle and two or more virus adaptor molecules such that the virus adaptor molecules bind to the virus particle, thereby generating a modified virus particle; and
   contacting a mixture of cells comprising the target cell with the modified virus particle, wherein the target cell expresses two or more different cell surface molecules, optionally wherein the virus adaptor molecules specifically bind to the two or more different cell surface molecules on the target cell.
196. A method of targeting a virus particle to two or more target cells comprising:
   providing a virus particle;
   providing two or more (e.g. 2, 3, 4 or 5) virus adaptor molecules comprising an isolated polypeptide capable of covalently binding to a virus capsid of the virus particle and a ligand specific to at least one of the two or more target cells;
   combining the virus particle and two or more virus adaptor molecules such that the virus adaptor molecules bind to the virus particle, thereby generating a modified virus particle; and

contacting a mixture of cells comprising the two or more target cells with the modified virus particle, wherein the two or more target cells express two or more different cell surface molecules, optionally wherein the virus adaptor molecules specifically bind to the different cell surface molecules on the two or more target cells.
197. The method of covalently modifying a virus particle or the method of targeting a virus particle to a target cell according to any one of embodiments 174 to 196, wherein the isolated polypeptide capable of covalently binding to a virus capsid comprises a portion of adeno-associated virus receptor (AAVR, KIAA0319L).
198. The method of covalently modifying a virus particle or the method of targeting a virus particle to a target cell according to any one of embodiments 174 to 197, wherein the isolated polypeptide capable of covalently binding to a virus capsid comprises a portion of adeno-associated virus receptor having one or more point mutations, optionally wherein the one or more point mutations are defined by reference to SEQ ID NO:1.
199. The method of covalently modifying a virus particle or the method of targeting a virus particle to a target cell according to any one of embodiments 174 to 198, wherein the isolated polypeptide capable of covalently binding to a virus capsid comprises a portion of adeno-associated virus receptor having one or more point mutations, optionally wherein the one or more point mutations are defined by reference to a sequence selected from the list consisting of: SEQ ID NO:2, SEQ ID NO:3, SEQ ID NO:4, SEQ ID NO:5 and SEQ ID NO:6.
200. The method of covalently modifying a virus particle or the method of targeting a virus particle to a target cell according to any one of embodiments 174 to 198, wherein the isolated polypeptide capable of covalently binding to a virus capsid comprises a V480E mutation in the PKD2 domain, optionally wherein the one or more point mutations are defined by reference to SEQ ID NO:1.
201. The method of covalently modifying a virus particle or the method of targeting a virus particle to a target cell according to any one of embodiments 174 to 199, wherein the isolated polypeptide capable of covalently binding to a virus capsid comprises the sequence of SEQ ID NO:1 with a V480E mutation.
202. The method of covalently modifying a virus particle or the method of targeting a virus particle to a target cell according to any one of embodiments 174 to 200, wherein the isolated polypeptide capable of covalently binding to a virus capsid comprises the sequence of SEQ ID NO:3 with a V480E mutation.
203. The method of covalently modifying a virus particle or the method of targeting a virus particle to a target cell according to any one of embodiments 174 to 202, wherein the isolated polypeptide capable of covalently binding to a virus capsid comprises a S425C mutation in the PKD2 domain, optionally wherein the one or more point mutations are defined by reference to SEQ ID NO:1.
204. The method of covalently modifying a virus particle or the method of targeting a virus particle to a target cell according to any one of embodiments 174 to 203, wherein the isolated polypeptide capable of covalently binding to a virus capsid comprises the sequence of SEQ ID NO:1 with a S425C mutation.
205. The method of covalently modifying a virus particle or the method of targeting a virus particle to a target cell according to any one of embodiments 174 to 204, wherein the isolated polypeptide capable of covalently binding to a virus capsid comprises the sequence of SEQ ID NO:3 with a S425C mutation.
206. The method of covalently modifying a virus particle or the method of targeting a virus particle to a target cell according to any one of embodiments 174 to 205, wherein the isolated polypeptide capable of covalently binding to a virus capsid comprises S425C and V480E mutations in the PKD2 domain, optionally wherein the one or more point mutations are defined by reference to SEQ ID NO:1 or SEQ ID NO:3.
207. The method of covalently modifying a virus particle or the method of targeting virus particle to a target cell according to any one of embodiments 174 to 206, wherein the isolated polypeptide capable of covalently binding to a virus capsid comprises at least a portion of the PKD1 domain, at least a portion of the PKD2 domain, at least a portion of the PKD3 domain, at least a portion of the PKD4 domain, at least a portion of the PKD5 domain of adeno-associated virus receptor (AAVR, KIAA0319L), or any combination thereof.
208. The method of covalently modifying a virus particle or the method of targeting virus particle to a target cell according to any one of embodiments 174 to 207, wherein the isolated polypeptide capable of covalently binding to a virus capsid comprises the PKD1 domain, the PKD2 domain, the PKD3 domain, the PKD4 domain, the PKD5 domain of adeno-associated virus receptor (AAVR, KIAA0319L), or any combination thereof.
209. The method of covalently modifying a virus particle or the method of targeting a virus particle to a target cell according to any one of embodiments 174 to 208, wherein the isolated polypeptide capable of covalently binding to an adeno associated virus (AAV) capsid comprises a sequence selected from the list consisting of: SEQ ID NO:2, SEQ ID NO:3, SEQ ID NO:4, SEQ ID NO:5 and SEQ ID NO:6.
210. The method of covalently modifying a virus particle or the method of targeting a virus particle to a target cell according to any one of embodiments 174 to 209, wherein the isolated polypeptide capable of covalently binding to a virus capsid comprises the full-length sequence of adeno-associated virus receptor (AAVR, KIAA0319L), optionally wherein the full sequence comprises SEQ ID NO:1.
211. A method of delivering a nucleic acid sequence of interest to a target cell comprising contacting the target cell with the modified virus particle according to any one of embodiments 142 to 172, or the pharmaceutical composition according to embodiment 173, wherein the virus particle comprises the nucleic acid sequence of interest.
212. The method of embodiment 211, wherein the nucleic acid sequence of interest encodes a protein selected from the list consisting of:
   RAB escort protein 1, RPE65, Factor VIII, Factor IX, Cochlin, CLN7, acid a-glucosidase (GAA), Aquaporin 1, Glial cell line-derived neurotrophic factor, aspartoacylase, Aromatic L-amino Acid Decarboxylase, Defects in Retinitis Pigmentosa GTPase Regulator, sarcoplasmic reticulum calcium ATPase, Cyclic nucleotide gated channel beta 3, neurturin, Galactosidase beta 1, Glucose-6-Phosphatase, Phenylalanine hydroxylase, Ornithine Transcarbamylase, Dystrophin and Carnitine palmitoyltransferase II, phenylalanine hydroxylase (PAH), Cystic fibrosis transmembrane conductance regulator (CFTR).
213. The method according to any one of embodiments 194 to 212, wherein the target cell is in vitro.
214. The method according to any one of embodiments 194 to 212, wherein the target cell is in vivo in a subject.
215. The method according to anyone of embodiments 194 to 214, wherein the target cell is selected from the list consisting of: neurons, macrophages, microglia, T-cells, B-cells, dendritic cells, antigen presenting cells, NK cells, cancer cells, muscle cells, hepatocytes, photoreceptors, pancreatic beta cells, kidney cells and lung cells.
216. The method according to embodiment 214 or 215, wherein the subject is a human.
217. The method according to any one of embodiments 194 to 216, wherein the target cell is a human target cell.
218. A virus particle according to any one of embodiments 142 to 172, or a pharmaceutical composition according to embodiment 173 for use as a medicament.
219. A virus particle according to any one of embodiments 142 to 172, or a pharmaceutical composition according to embodiment 173 for use in a method of treating a disease in a subject in need thereof, the method comprising delivering a nucleic acid sequence of interest to a target cell by contacting the target cell with the virus particle.
220. The virus particle or pharmaceutical composition for use according to embodiment 219, wherein the disease is selected from the list consisting of:
   a neurodegenerative disorder, cancer, Duchenne muscular dystrophy, haemophilia, a congenital blindness disorder, diabetes, cystic fibrosis, choroideremia, hemophilia A, hemophilia B, CLN7 disease, Pompe disease, Parkinson's disease, Canavan disease, demyelinating diseases, inherited retinal dystrophy due to RPE65 mutations, aromatic L-amino acid decarboxylase (AADC) deficiency, X-linked retinitis pigmentosa, Leber congenital amaurosis, Churg-Strauss Syndrome (CSS), critical limb ischemia, achromatopsia, Alzheimer's disease, macular degeneration, ornithine transcarbamylase deficiency, Wilson disease, glycogen storage disease type IA, Crigler Najjar syndrome, Tay-Sachs disease, Sandhoff disease, multiple myeloma, multiple system atrophy, gangliosidosis, Danon disease, Fabry disease, Batten disease, phenylketonuria, rheumatoid arthritis, mucopolysaccharidosis type IIIa, Sanfilippo syndrome B, mucopolysaccharidosis type VI, alpha 1-antitrypsin deficiency, spinal muscular atrophy type 1, Krabbe disease, Becker muscular dystrophy, Charcot-Marie-Tooth neuropathy type 1a, carnitine palmitoyltransferase II (CPT II) deficiency and trimethylaminuria, cystic fibrosis, phenylketonuria.
221. A virus particle according to any one of embodiments 142 to 172, or a pharmaceutical composition according to embodiment 173 for use in a method of treating a disease in a subject in need thereof, the method comprising delivering a nucleic acid sequence of interest to a target cell by contacting the target cell with the virus particle, wherein the targeting of the specified target cell treats or ameliorates the specified disease according to the following list:
   a) neurons for treatment of neurodegenerative disorders;
   b) immune cells (e.g. macrophages, microglia, T-cells, B-cells, dendritic cells, antigen presenting cells, NK cells) for treatment of cancer;
   c) immune cells (e.g. macrophages, microglia, T-cells, B-cells, dendritic cells, antigen presenting cells, NK cells) to elicit an immune response;
   d) cancer or tumour cells for treatment of cancer;
   e) muscle cells for treatment of Duchenne muscular dystrophy;
   f) hepatocytes for treatment of haemophilia;
   g) photoreceptor cells for congenital blindness disorders;
   h) pancreatic beta cells for treatment of diabetes; or
   i) lung cells for treatment of cystic fibrosis.
222. A virus particle or a pharmaceutical composition for use according to any one of embodiments 218 to 221 wherein the virus particle or a pharmaceutical composition is administered to the subject via aerosol (e.g to lung cells), intramuscularly, intraarterially (e.g. via the hepatic artery), intraarticularly, subretinally, intracranially, intravenously, intrathecally. intra-coronarily or subcutaneously.
223. A virus particle covalently bound to at least one isolated polypeptide or virus adaptor molecule wherein the isolated polypeptide or virus adaptor molecule and the virus have an affinity (Kd) of less than 100 μM.
224. The virus particle of embodiment 223 wherein at least one pair of residues X and Y exist, where X is part of the isolated polypeptide or virus adaptor molecule and Y is part of the virus particle, such that their respective β carbons are less than 5.5 Å apart when the isolated polypeptide or virus adaptor molecule forms a complex with the virus particle.
225. The virus particle of embodiment 224 wherein residues X and Y can be mutated to cysteines such that the cysteine side chains do not induce a major steric clash which would disrupt the interface.
226. The virus particle of embodiment 224 or 225 wherein an orientation exists for the side chains of residues X and Y, after mutation to cysteines, obtained by rotation of the side chain around C(α)-C(β), such that the distance of the sulfur atoms at the gamma positions of the side chains are less than 2.5 Å apart.
227. A virus particle covalently bound to at least one isolated polypeptide or virus adaptor molecule wherein at least one pair of residues X and Y exist, where X is part of the isolated polypeptide or virus adaptor molecule and Y is part of the virus particle, such that their respective β carbons are less than 5.5 Å apart when the isolated polypeptide or virus adaptor molecule forms a complex with the virus particle.
228. The virus particle of embodiment 227 wherein residues X and Y can be mutated to cysteines such that the cysteine side chains do not induce a major steric clash which would disrupt the interface.
229. The virus particle of embodiment 227 or 228 wherein an orientation exists for the side chains of residues X and Y, after mutation to cysteines, obtained by rotation of the side chain around C(α)-C(β), such that the distance of the sulfur atoms at the gamma positions of the side chains are less than 2.5 Å apart.
230. The virus particle of any one of embodiments 223-229, wherein the angles formed by the atoms the C(β)1-S(γ)1-S(γ)2, S(γ)1-S(γ)2-C(β) would not be markedly different (for example are not more than 40% greater) than the optimal angles observed in naturally occurring disulfide bonds.
231. A virus particle covalently bound to at least one isolated polypeptide or virus adaptor molecule wherein the angles formed by the atoms the C(β)1-S(γ)1-S(γ)2, S(γ)1-S(γ)2-C(β) would not be markedly different (for example are not more than 40% greater) than the optimal angles observed in naturally occurring disulfide bonds.
232. A virus-like particle (VLP) bound to at least one isolated polypeptide or AAV adaptor molecule according to any one of embodiments 1 to 31 or at least one isolated polypeptide or virus adaptor molecule according to any one of embodiments 98 to 141.
233. A virus-like particle (VLP) bound to at least 2, 3, 4 or 5 isolated polypeptides or AAV adaptor molecules according to any one of embodiments 1 to 31 or at least one isolated polypeptide or virus adaptor molecule according to any one of embodiments 98 to 141.
234. The virus-like particle (VLP) according to embodiment 233, wherein the virus-like particle has specificity for two or more different target cells.
235. The virus-like particle (VLP) according to embodiment 233, wherein the virus-like particle has increased tropism for two or more different target cells.
236. The virus-like particle (VLP) according to embodiment 234 or 235, wherein the two or more target cells express different cell surface molecules.
237. The virus-like particle (VLP) according to any one of embodiments 233 to 235, wherein the at least 2, 3, 4 or 5 isolated polypeptides or AAV adaptor molecules specifically bind to the different cell surface molecules on the two or more target cells.
238. The virus-like particle (VLP) according to embodiment 233, wherein the virus-like particle has specificity for a target cell expressing two or more different cell surface molecules.
239. The virus-like particle (VLP) according to embodiment 233, wherein the virus-like particle has increased tropism for a target cell expressing two or more different cell surface molecules.
240. The virus-like particle (VLP) according to embodiments 238 or 239, wherein the at least 2, 3, 4 or 5 isolated polypeptides or AAV adaptor molecules specifically bind to the two or more different cell surface molecules on the target cell.
241. The virus-like particle (VLP) according to any one of embodiments 232 to 240, wherein the virus-like particle comprises a portion of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
242. The virus-like particle (VLP) according to any one of embodiments 232 to 241, wherein the virus-like particle comprises the full-length of a capsid amino acid sequence selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
243. The virus-like particle (VLP) according to any one of embodiments 232 to 242, wherein the virus-like particle capsid proteins are selected from one or more viruses in the list consisting of: Adenovirus, Aichi virus, Australian bat lyssavirus, BK polyomavirus, Banna virus, Barmah forest virus, Bunyamwera virus, Bunyavirus, La CrosseBunyavirus, snowshoe hare Cercopithecine herpesvirus, Chandipura virus, Chikungunya virus, Cosavirus, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus, C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus, Human herpesvirus, Human herpesvirus, Human immunodeficiency virus, Human papillomavirus, Human papillomavirus, Human parainfluenza, Human parvovirus, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephali-tis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New Jersey polyomavirus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus, ARoss river virus, Rotavirus, ARotavirus, BRotavirus, CRubella virus, Sagiyama virus, Salivirus, ASandfly fever sicilian virus, Sapporo virus, SARS coronavirus, Semliki forest virus, Seoul virus, Simian foamy virus, Simian virus 40, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus and Zika virus.
244. The virus-like particle (VLP) according to any one of embodiments 232 to 243, wherein the virus-like particle has one or more conservative amino acid changes relative to a wildtype viral capsid amino acid sequence, for example 1, 2, 3, 4, 5, 6, 7, 8, 9, 10, 11, 12, 13, 14, 15, 16, 17, 18, 19 or 20 conservative amino acid changes relative to a wildtype viral capsid amino acid sequence, optionally wherein the wildtype viral capsid amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
245. The virus-like particle (VLP) according to any one of embodiments 232 to 243, wherein the virus-like particle has five or fewer non-conservative amino acid changes relative to the wildtype viral capsid amino acid sequence, for example 5, 4, 3, 2 or 1 non-conservative amino acid changes relative to the wildtype viral capsid amino acid sequence, optionally wherein the wildtype viral capsid amino acid sequence is selected from the group consisting of SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18 and SEQ ID NO:19.
246. The virus-like particle (VLP) according to any one of embodiments 232 to 245, wherein the virus-like particle comprises one or more amino acid changes in a capsid protein that mediates the interaction with non-protein binders such as heparan sulfate proteoglycan (HSPG), O-linked sialic acid, N-linked sialic acid, N-linked galactose.
247. The virus-like particle (VLP) according to embodiment 246, wherein the one or more mutations comprise arginine residues 585 and 588 of AAV2.
248. A pharmaceutical composition comprising a virus-like particle (VLP) according to any one of embodiments 232 to 247.
249. A method of modifying a virus-like particle comprising:
   providing a virus-like particle;
   providing an isolated polypeptide capable of binding to a virus-like particle capsid; and
   combining the virus-like particle and the isolated polypeptide such that the isolated polypeptide binds to the virus-like particle.
250. A method of modifying a virus-like particle comprising:
   providing a virus-like particle;
   providing an AAV adaptor molecule comprising an isolated polypeptide capable of binding to a virus-like particle capsid and a ligand; and
   combining the virus-like particle and the AAV adaptor molecule such that the isolated fusion polypeptide binds to the virus-like particle.
251. A method of modifying a virus-like particle comprising:
   providing a virus-like particle;
   providing two or more (e.g. 2, 3, 4 or 5) isolated polypeptides capable of binding to a virus-like particle capsid; and
   combining the virus-like particle and the isolated polypeptides such that the isolated polypeptides bind to the virus-like particle.
252. A method of modifying a virus-like particle comprising:
   providing a virus-like particle;
   providing two or more (e.g. 2, 3, 4 or 5) AAV adaptor molecules comprising an isolated polypeptide capable of binding to a virus-like particle capsid and a ligand; and
   combining the virus-like particle and the AAV adaptor molecules such that the AAV adaptor molecules bind to the virus-like particle.
253. The method of modifying a virus-like particle according to any one of embodiments 249 to 252, wherein the binding of the isolated polypeptide or AAV adaptor molecule to the AAV particle reduces or abolishes the natural tropism of one or more AAV capsid proteins.
254. The method of modifying a virus-like particle according to any one of embodiments 249 to 253, wherein the binding of the isolated polypeptide or AAV adaptor molecule to the AAV particle increases the tropism of the AAV particle for one or more cell types.
255. A method of targeting a virus-like particle to a target cell comprising:
   providing a virus-like particle;
   providing an AAV adaptor molecule comprising an isolated polypeptide capable of binding to a virus-like particle capsid and a ligand specific to the target cell; combining the virus-like particle and the AAV adaptor molecule such that the AAV adaptor molecule binds to the virus-like particle, thereby generating a virus-like particle; and
   contacting a mixture of cells comprising the target cell with the modified virus-like particle.
256. A method of targeting a virus-like particle to a target cell comprising:
   providing a virus-like particle;
   providing two or more (e.g. 2, 3, 4 or 5) AAV adaptor molecules comprising an isolated polypeptide capable of binding to a virus-like particle capsid and a ligand specific to the target cell;
   combining the virus-like particle and two or more AAV adaptor molecules such that the AAV adaptor molecules bind to the virus-like particle, thereby generating a modified virus-like particle; and
   contacting a mixture of cells comprising the target cell with the modified virus-like particle, wherein the target cell expresses two or more different cell surface molecules, optionally wherein the AAV adaptor molecules specifically bind to the two or more different cell surface molecules on the target cell.
257. A method of targeting a virus-like particle to two or more target cells comprising: providing a virus-like particle;
   providing two or more (e.g. 2, 3, 4 or 5) AAV adaptor molecules comprising an isolated polypeptide capable of binding to a virus-like particle capsid and a ligand specific to at least one of the two or more target cells;
   combining the virus-like particle and two or more AAV adaptor molecules such that the AAV adaptor molecules bind to the virus-like particle, thereby generating a modified virus-like particle; and
   contacting a mixture of cells comprising the two or more target cells with the modified virus-like particle, wherein the two or more target cells express two or more different cell surface molecules, optionally wherein the AAV adaptor molecules specifically bind to the different cell surface molecules on the two or more target cells.
258. A method of covalently modifying a virus-like particle comprising:
   providing a virus-like particle;
   providing an isolated polypeptide capable of covalently binding to a virus capsid of the virus-like particle; and
   combining the virus-like particle and the isolated polypeptide such that the isolated polypeptide covalently binds to the virus-like particle.
259. A method of covalently modifying a virus-like particle comprising:
   providing a virus-like particle;
   providing a virus adaptor molecule comprising an isolated polypeptide capable of covalently binding to a virus capsid of the virus-like particle and a ligand; and
   combining the virus-like particle and the virus adaptor molecule such that the virus adaptor molecule covalently binds to the virus-like particle.
260. A method of covalently modifying a virus particle comprising:
   providing a virus-like particle;
   providing two or more (e.g. 2, 3, 4 or 5) isolated polypeptides capable of covalently binding to a virus capsid of the virus-like particle; and
   combining the virus-like particle and the two or more isolated polypeptides such that the isolated polypeptides covalently bind to the virus-like particle.
261. A method of covalently modifying a virus-like particle comprising:
   providing a virus-like particle;
   providing two or more (e.g. 2, 3, 4 or 5) virus adaptor molecules comprising an isolated polypeptide capable of covalently binding to a virus capsid of the virus-like particle and a ligand; and
   combining the virus-like particle and the two or more virus adaptor molecules such that the virus adaptor molecules covalently bind to the virus-like particle.
262. The method of covalently modifying a virus-like particle according to any one of embodiments 258 to 262, wherein the method further comprises introducing one or more heterologous cysteine residues to the isolated polypeptide or virus adaptor molecule, for example 1, 2 or 3 heterologous cysteine residues.
263. The method of covalently modifying a virus-like particle according to any one of embodiments 258 to 263, wherein the method further comprises introducing one or more heterologous cysteine residues to a virus capsid of the virus-like particle, for example 1, 2 or 3 heterologous cysteine residues.

1-25. (canceled)

26. An isolated polypeptide comprising one or more cysteine residues capable of covalently binding to one or more cysteine residues on a viral capsid having 5 or fewer amino acid point mutations compared to a wildtype viral capsid at the interface between the isolated polypeptide and the virus capsid,
wherein the isolated polypeptide has an affinity Kd<100 μM for the viral capsid and wherein the respective R carbons of the one or more cysteine residues of the isolated polypeptide and the one or more cysteine residues of the viral capsid are less than 5.5 Å apart when the isolated polypeptide forms a complex with the viral capsid.

27. The isolated polypeptide according to claim 26 wherein the one or more cysteine residues capable of covalently binding to a viral capsid are heterologous to the isolated polypeptide.

28. A virus adaptor molecule comprising a fusion protein comprising (i) an isolated polypeptide according to claim 26 or claim 27 and (ii) a ligand capable of binding to a cell surface molecule.

29. The virus adaptor molecule according to claim 28 comprising (i) an isolated polypeptide comprising one or more cysteine residues capable of covalently binding to a viral capsid and (ii) a ligand capable of binding to a cell surface molecule, wherein the one or more cysteine residues are heterologous to the isolated polypeptide.

30. The isolated polypeptide or virus adaptor molecule according to any one of claims 26 to 29, wherein the isolated polypeptide or virus adaptor molecule comprises a portion of adeno-associated virus receptor (AAVR, KIAA0319L) having one or more heterologous cysteine residues capable of forming covalent disulfide bonds with an adeno-associated virus capsid.

31. The isolated polypeptide or virus adaptor molecule according to any one of claims 26 to 29, wherein the isolated polypeptide or covalent binding virus adaptor molecule comprises a portion of a coxsackievirus-adenovirus receptor (CXADR) having one or more heterologous cysteine residues capable of forming covalent disulfide bonds with a viral capsid, optionally wherein the CXADR sequence is SEQ ID NO:20.

32. The isolated polypeptide or virus adaptor molecule according to any one of claims 26 to 29, wherein the isolated polypeptide or covalent binding virus adaptor molecule comprises one or more complementarity determining regions (CDRs) of neutralizing antibody A20.

33. The virus adaptor molecule according to any one of claims 28 to 32, wherein the ligand binds one or more cell surface molecules selected from the list consisting of:
Her2, Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-8 receptor, Interleukin-9 receptor, Interleukin-10 receptor, Interleukin-11 receptor, Interleukin-12 receptor, Interleukin-13 receptor, Interleukin-15 receptor, Interleukin-18 receptor, Interleukin-20 receptor, Interleukin-21 receptor, Interleukin-22 receptor, Interleukin-23 receptor, Interleukin-27 receptor, Interleukin-28 receptor, Insulin receptor, Transferrin receptor, CD58, CD2, CD2, CD59, CD40, CD72, CD5, CD36, CD19, CD21, CD81, CD27, CD28, CTLA-4, CD85j, CD95, CD96, α4β1 integrin, CD115, CD6, CD178, LFA-1, TNFRSF4, DR4, DR5, RANK/CD265, TACI/CD267, CD267, CD268, CD269, HVEM, PD1/CD279, B7-1/CD80, CD278, CD4, CD8, CD19, NMDAR, AMPAR, mGluR5, DRD1, DRD2, Bmp4, GLP1R, leptin receptor, α5β5 integrin and glycoRNAs.

34. The isolated polypeptide or virus adaptor molecule according to any one of claims 30 to 33, wherein the portion of adeno-associated virus receptor (AAVR, KIAA0319L) comprises at least 80% of the full-length of the PKD1 domain, at least 80% of the full-length of the PKD2 domain, at least 80% of the full-length of the PKD3 domain, at least 80% of the full-length of the PKD4 domain, at least 80% of the full-length of the PKD5 domain, or any combination thereof.

35. A virus particle covalently bound to at least one isolated polypeptide or virus adaptor molecule according to any one of claims 26 to 34.

36. The virus particle according to claim 35, wherein the virus is an adeno-associated virus, optionally wherein the adeno associated virus is selected from the list consisting of: AAV1, AAV2, AAV3, AAV4, AAV5, AAV6, AAV7, AAV8, AAV9, AAV10, AAV11, AAV12, AAV13, AAVrh.8, AAVrh.10, and AAVrh.74.

37. The virus particle according to any one of claim 35 or 36, where in the virus particle comprises an unmodified, wildtype viral capsid.

38. The virus particle according to any one of claim 35 or 36, where in the virus particle comprises a viral capsid having 5 or fewer amino acid point mutations compared to a wildtype viral capsid.

39. The virus particle according to any one of claim 35 or 36, where in the virus particle comprises a viral capsid having an amino acid insertion or deletion of no more than 5 amino acids relative to a wildtype viral capsid.

40. A pharmaceutical composition comprising a virus particle according to any one of claim 35 to 39.

41. A method of covalently modifying a virus particle comprising:
providing a virus particle;
providing an isolated polypeptide capable of covalently binding to a virus capsid of the virus particle according to any one of claims 26, 27 or 29 to 34; and
combining the virus particle and the isolated polypeptide such that the isolated polypeptide covalently binds to the virus particle.

42. A method of covalently modifying a virus particle comprising:
providing a virus particle;
providing a virus adaptor molecule comprising an isolated fusion polypeptide capable of covalently binding to a virus capsid of the virus particle and a ligand capable of binding a cell surface molecule according to any one of claims 28 to 34; and
combining the virus particle and the virus adaptor molecule such that the isolated fusion polypeptide covalently binds to the virus particle.

43. The method of covalently modifying a virus particle according to claim 41 or claim 42, wherein the method further comprises introducing one or more heterologous cysteine residues to the isolated polypeptide or virus adaptor molecule, for example 1, 2 or 3 heterologous cysteine residues.

44. The method of covalently modifying a virus particle according to any one of claims 41 to 43, wherein the covalent binding of the isolated polypeptide or virus adaptor molecule to the virus particle reduces by at least 50% or abolishes the natural tropism of one or more virus capsid proteins.

45. The method of covalently modifying a virus particle according to any one of claims 41 to 43, wherein the covalent binding of the isolated polypeptide or virus adaptor molecule to the virus particle increases the tropism of the virus particle for one or more cell types by at least 50%.

46. A virus particle according to any one of claims 35 to 39, or a pharmaceutical composition according to claim 40 for use as a medicament.

47. A virus particle according to any one of claims 35 to 39, or a pharmaceutical composition according to claim 40 for use in a method of treating a disease in a subject in need thereof, the method comprising delivering a nucleic acid sequence of interest to a target cell by contacting the target cell with the virus particle.


SEQUENCE LISTING (31 sequences)

SEQ ID NO: 1 - protein, 1049 aa, synthetic construct
MEKRLGVKPNPASWILSGYYWQTSAKWLRSLYLFYTCFCFSVLWLSTDASESRCQQGKTQFGVGLRSGGENHLWLLEGTPSLQSCWAACCQDSACHVFWWLEGMCIQADCSRPQSCRAFRTHSSNSMLVFLKKFQTADDLGFLPEDDVPHLLGLGWNWASWRQSPPRAALRPAVSSSDQQSLIRKLQKRGSPSDVVTPIVTQHSKVNDSNELGGLTTSGSAEVHKAITISSPLTTDLTAELSGGPKNVSVQPEISEGLATTPSTQQVKSSEKTQIAVPQPVAPSYSYATPTPQASFQSTSAPYPVIKELVVSAGESVQITLPKNEVQLNAYVLQEPPKGETYTYDWQLITHPRDYSGEMEGKHSQILKLSKLTPGLYEFKVIVEGQNAHGEGYVNVTVKPEPRKNRPPIAIVSPQFQEISLPTTSTVIDGSQSTDDDKIVQYHWEELKGPLREEKISEDTAILKLSKLVPGNYTFSLTVVDSDGATNSTTANLTVNKAVDYPPVANAGPNQVITLPQNSITLFGNQSTDDHGITSYEWSLSPSSKGKVVEMQGVRTPTLQLSAMQEGDYTYQLTVTDTIGQQATAQVTVIVQPENNKPPQADAGPDKELTLPVDSTTLDGSKSSDDQKIISYLWEKTQGPDGVQLENANSSVATVTGLQVGTYVFTLTVKDERNLQSQSSVNVIVKEEINKPPIAKITGNVVITLPTSTAELDGSKSSDDKGIVSYLWTRDEGSPAAGEVLNHSDHHPILFLSNLVEGTYTFHLKVTDAKGESDTDRTTVEVKPDPRKNNLVEIILDINVSQLTERLKGMFIRQIGVLLGVLDSDIIVQKIQPYTEQSTKMVFFVQNEPPHQIFKGHEVAAMLKSELRKQKADFLIFRALEVNTVTCQLNCSDHGHCDSFTKRCICDPFWMENFIKVQLRDGDSNCEWSVLYVIIATFVIVVALGILSWTVICCCKRQKGKPKRKSKYKILDATDQESLELKPTSRAGIKQKGLLLSSSLMHSESELDSDDAIFTWPDREKGKLLHGQNGSVPNGQTPLKARSPREEIL

SEQ ID NO: 2 - protein, 90 aa, synthetic construct
SAGESVQITLPKNEVQLNAYVLQEPPKGETYTYDWQLITHPRDYSGEMEGKHSQILKLSKLTPGLYEFKVIVEGQNAHGEGYVNVTVKPE

SEQ ID NO: 3 - protein, 90 aa, synthetic construct
IAIVSPQFQEISLPTTSTVIDGSQSTDDDKIVQYHWEELKGPLREEKISEDTAILKLSKLVPGNYTFSLTVVDSDGATNSTTANLTVNKA

SEQ ID NO: 4 - protein, 91 aa, synthetic construct
VANAGPNQVITLPQNSITLFGNQSTDDHGITSYEWSLSPSSKGKVVEMQGVRTPTLQLSAMQEGDYTYQLTVTDTIGQQATAQVTVIVQPE

SEQ ID NO: 5 - protein, 89 aa, synthetic construct
QADAGPDKELTLPVDSTTLDGSKSSDDQKIISYLWEKTQGPDGVQLENANSSVATVTGLQVGTYVFTLTVKDERNLQSQSSVNVIVKEE

SEQ ID NO: 6 - protein, 92 aa, synthetic construct
IAKITGNVVITLPTSTAELDGSKSSDDKGIVSYLWTRDEGSPAAGEVLNHSDHHPILFLSNLVEGTYTFHLKVTDAKGESDTDRTTVEVKPD

SEQ ID NO: 7 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 735 aa, synthetic construct [sequence omitted]

SEQ ID NO: 9 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 11 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 13 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 14 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 15 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 17 - protein, 733 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 18 - protein, 742 aa, synthetic construct
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 19 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 20 - protein, 365 aa, synthetic construct
MALLLCFVLLCGVVDFARSLSITTPEEMIEKAKGETAYLPCKFTLSPEDQGPLDIEWLISPADNQKVDQVIILYSGDKIYDDYYPDLKGRVHFTSNDLKSGDASINVTNLQLSDIGTYQCKVKKAPGVANKKIHLVVLVKPSGARCYVDGSEEIGSDFKIKCEPKEGSLPLQYEWQKLSDSQKMPTSWLAEMTSSVISVKNASSEYSGTYSCTVRNRVGSDQCLLRLNVVPPSNKAGLIAGAIIGTLLALALIGLIIFCCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 21 - protein, 107 aa, synthetic construct
  REGION 1..107: note = A20 variable light chain
DIQMTQSSSSFSVSLGDRVTITCKASEDIHNRLAWYKQKPGNAPRLLISGATSLETGVPSRFSGSGSGKDYTLSITSLQNEDVATYYCQQYWIGPFTFGSGTNLEIK

SEQ ID NO: 22 - protein, 11 aa, synthetic construct
  REGION 1..11: note = A20 VLCDR1
KASEDIHNRLA

SEQ ID NO: 23 - protein, 8 aa, synthetic construct
  REGION 1..8: note = A20 VLCDR2
SGATSLET

SEQ ID NO: 24 - protein, 9 aa, synthetic construct
  REGION 1..9: note = A20 VLCDR3
QQYWIGPFT

SEQ ID NO: 25 - protein, 120 aa, synthetic construct
  REGION 1..120: note = A20 variable heavy chain
SDVQLQESGPDLVKPSQSLSLTCTVTGYSITSGYTWHWIRQFPGNKQEWMGYIHFSGYTNYNPSLKSRVSITRDTSKNQFFLHLNSVTTEDTATYYCARGDYGYEWFTYWGQGTLVTVSA

SEQ ID NO: 26 - protein, 14 aa, synthetic construct
  REGION 1..14: note = A20 VHCDR1
TVTGYSITSGYTWH

SEQ ID NO: 27 - protein, 9 aa, synthetic construct
  REGION 1..9: note = A20 VHCDR2
YIHFSGYTN

SEQ ID NO: 28 - protein, 12 aa, synthetic construct
  REGION 1..12: note = A20 VHCDR3
ARGDYGYEWFTY

SEQ ID NO: 29 - protein, 392 aa, synthetic construct [sequence omitted]

SEQ ID NO: 30 - protein, 319 aa, synthetic construct
MAKRARLSTSFNPVYPYEDESSSQHPFINPGFISPDGFTQSPNGVLSLKCVNPLTTASGSLQLKVGSGLTVDTTDGSLEENIKVNTPLTKSNHSINLPIGNGLQIEQNKLCSKLGNGLTFDSSNSIALKNNTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 31 - protein, 1118 aa, synthetic construct
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYS